SEQUENCE LISTING (2 sequences)

SEQ ID NO: 1 - protein, 30 aa, Artificial
  Synthetically Generated Sequence
GLARFSRFAGSSPSQSSMVARTQTVRGTLA

SEQ ID NO: 2 - protein, 357 aa, Homo sapiens
MSYYHHHHHHDYDIPTTENLYFQGAMGDRTLMTPVQNLEQSYMPPDSSSPENKSLEVSDTRFHSFSFYELKNVTNNFDERPISVGGNKMGEGGFGVVYKGYVNNTTVAVKKLAAMVDITTEELKQQFDQEIKVMAKCQHENLVELLGFSSDGDDLCLVYVYMPNGSLLDRLSCLDGTPPLSWHMRCKIAQGAANGINFLHENHHIHRDIKSANILLDEAFTAKISDFGLARASEKFAQTVMTSRIVGTTAYMAPEALRGEITPKSDIYSFGVVLLEIITGLPAVDEHREPQLLLDIKEEIEDEEKTIEDYIDKKMNDADSTSVEAMYSVASQCLHEKKNKRPDIKKVQQLLQEMTAS

(19) United States
(12) Patent Application Publication
(10) Pub. No.: US 2011/0021513 A1
(43) Pub. Date: Jan. 27, 2011

(54) MODULATORS OF INTERLEUKIN-1 RECEPTOR-ASSOCIATED KINASE

(75) Inventors: Thomas Durand-Reville, Cambridge, MA (US); Charles Jewell, Sudbury, MA (US); Charles Hammond, Burlington, MA (US); Donovan Chin, Lexington, MA (US)

Correspondence Address: STEPTOE & JOHNSON LLP, 1330 CONNECTICUT AVE., NW, WASHINGTON, DC, 20036, US

(73) Assignee: Biogen Idec Ma Inc., Cambridge, MA (US)

(21) Appl. No.: 12/440,154

(22) PCT Filed: Sep. 7, 2007
(86) PCT No.: PCT/US2007/019577

§ 371 Date: Oct. 15, 2010

Related U.S. Application Data
(60) Provisional application No. 60/842,800, filed on Sep. 7, 2006.

(51) Int. Cl. A61K 31/538; A61K 31/5377; A61K 31/5025; C07D 487/04; A61P 29/00; A61P 37/00; A61P 19/02; A61P 19/10; A61P 9/10; A61P 25/28; A61P 17/06; A61P 25/16; A61P 3/10; A61P 31/04

(52) U.S. Cl. 514/230.5; 514/233.2; 514/248; 544/105; 544/117; 544/236

(57) ABSTRACT

The present invention relates to modulators of IRAK kinase and provides compositions comprising such modulators, as well as methods therewith for treating conditions or diseases mediated by or associated with IRAK kinase.

### CROSS-REFERENCE

This application claims priority to U.S. Application No. 60/842,800, filed Sep. 7, 2006.

### TECHNICAL FIELD OF THE INVENTION

The present invention is directed to compounds which are capable of modulating (e.g., activating or inhibiting) interleukin-1 (IL-1) receptor-associated kinase (IRAK) and thus are useful in the prevention or treatment of conditions or diseases associated or mediated by IRAK, e.g., some inflammatory, cell proliferative and immune-related conditions or diseases. The invention is also directed to pharmaceutical compositions containing these compounds and the use of these compounds and pharmaceutical compositions in the prevention or treatment of conditions or diseases associated or mediated by IRAK.

### BACKGROUND OF THE INVENTION

The recruitment of immune cells to sites of injury involves the concerted interactions of a large number of soluble mediators. Several cytokines appear to play key roles in these processes, particularly IL-1 and tumor necrosis factor (TNF). Both cytokines are derived from mononuclear cells and macrophages, along with other cell types. Physiologically, they produce many of the same proinflammatory responses, including fever, sleep and anorexia, mobilization and activation of polymorphonuclear leukocytes, induction of cyclooxygenase and lipoxygenase enzymes, increase in adhesion molecule expression, activation of B-cells, T-cells and natural killer cells, and stimulation of production of other cytokines. Other actions include contribution to the tissue degeneration observed in chronic inflammatory conditions, such as stimulation of fibroblast proliferation, induction of collagenase, etc. They have also been implicated in the process of bone resorption and adipose tissue regulation. Thus, these cytokines play key roles in a large number of pathological conditions, e.g., rheumatoid arthritis, inflammatory bowel disease, multiple sclerosis, diabetes, obesity, cancer, sepsis, osteoarthritis, osteoporosis, myasthenia gravis, stroke, Alzheimer's disease, Parkinson's disease, amyotrophic lateral sclerosis, psoriasis, cardiac contractile dysfunction, type I diabetes, type II diabetes, familial cold autoinflammatory syndrome, severe bacterial infections (which may cause, e.g., apoptosis of macrophages, such as anthrax, bubonic plague and typhoid fever).

The importance of IL-1 in inflammation has been demonstrated by the ability of the highly specific IL-1 receptor antagonist protein (IL-1Ra or IRAP) to relieve inflammatory conditions. See, e.g., Dinarello, Cytokine Growth Factor Rev., 1997, 8: 253-265.

IL-1 treatment of cells induces the formation of a complex consisting of the two IL-1 receptor chains, IL-1R1 and IL-1RAcP, and the resulting heterodimer recruits an adaptor molecule designated as MyD88. See, e.g., Wesche et al., J. Biol. Chem., 1999, 274: 19403-19410. MyD88 binds to a protein designated IRAK (IL-1 receptor associated kinase). See, e.g., O'Neill et al., J. Leukoc. Biol., 1998, 63(6):650-657; Auron, Cytokine Growth Factor Rev., 1998, 9(3-4): 221-237; and O'Neill, Biochem. Soc. Trans., 2000, 28(5): 557-563. IRAK is subsequently phosphorylated and released from the receptor complex to interact with a tumor necrosis factor receptor-associated factor, TRAF6, which transduces the signal to downstream effector molecules. See, e.g., Cao et al., Nature, 1996, 383: 443-446. TRAF6 can trigger the NIK/IKK kinase cascade to activate the transcription factor NF-κB. NF-κB regulates a number of genes that, in turn, regulate immune and inflammatory responses.

Four IRAKs have been identified: IRAK-1 (see, e.g., Cao et al., Science, 1996, 271: 1128-1131), IRAK-2 (see, e.g., Muzio et al., Science, 1997, 278: 1612-1615), the monomyeloic cell-specific IRAK-M, also known as IRAK-3 (see, e.g., Wesche et al., J. Biol. Chem., 1999, 274: 19403-10), and IRAK-4 (see, e.g., PCT Publication No. WO 01/051641). IRAK proteins have been shown to play a role in transducing signals other than those originating from IL-1 receptors, including signals triggered by activation of IL-18 receptors (see, e.g., Kanakaraj et al., J. Exp. Med., 1999, 189(7): 1129-1138) and LPS receptors (see, e.g., Yang et al., J. Immunol., 1999, 163: 639-643; and Wesche et al., J. Biol. Chem., 1999, 274: 19403-19410). Over-expression of IRAK-2 and IRAK-M has been shown to be capable of reconstituting the response to IL-1 and LPS in an IRAK deficient cell line.

The identification of compounds that modulate the function of IRAK proteins represents an attractive approach to the development of therapeutic agents for the treatment of inflammatory, cell proliferative and immune-related conditions and diseases associated with IRAK-mediated signal transduction, such as rheumatoid arthritis, inflammatory bowel disease, multiple sclerosis, diabetes, obesity, allergic disease, psoriasis, asthma, graft rejection, cancer, and sepsis.

### SUMMARY OF THE INVENTION

In one aspect, the present invention provides a method of treating an inflammatory condition, a cell proliferative disorder, or an immune disorder, comprising administering to a subject in need of such treatment a therapeutically effective amount of a compound of Formula (I)

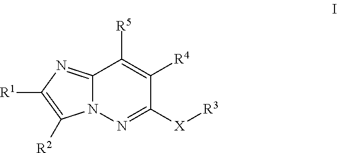

or a pharmaceutically acceptable salt thereof.

Referring to Formula (I),

each of R¹, R², R⁴, and R⁵ is independently H, halo, an amino, an optionally substituted aliphatic, an optionally substituted cycloaliphatic, an optionally substituted heterocycloaliphatic, an optionally substituted aryl, or an optionally substituted heteroaryl;

R³ is H, an optionally substituted aliphatic, an optionally substituted cycloaliphatic, an optionally substituted heterocycloaliphatic, an optionally substituted aryl, or an optionally substituted heteroaryl;

X is O, C(O), N(R) or S(O)ₙ;

n is 0, 1, or 2; and

R is H, an optionally substituted aliphatic, an optionally substituted cycloaliphatic, an optionally substituted heterocycloaliphatic, an optionally substituted aryl, or an optionally substituted heteroaryl; or

when X is N(R), R³ and R, together with the nitrogen atom to which they are attached, may form a 3- to 7-membered optionally substituted heterocycloaliphatic or heteroaryl ring, which may contain additional hetero ring atoms selected from O, S, or N, in addition to the nitrogen atom to which R³ and R are attached.

In some embodiments, R³ is an optionally substituted aliphatic.

In some embodiments, R³ is an aliphatic optionally substituted with an optionally substituted aryl or an optionally substituted heteroaryl.

In further embodiments, R³ is an aliphatic optionally substituted with halo, amino, hydroxy, oxo, alkoxy (e.g., of 1 to 4 or 1 to 6 carbon atoms), sulfonamide, cyano, nitro, an optionally substituted cycloaliphatic, or an optionally substituted heterocycloaliphatic.

In other embodiments, R³ is an optionally substituted arylaliphatic or optionally substituted heteroaryl(aliphatic), in which the aryl or heteroaryl substituent is further optionally substituted, e.g., with 1 to 6 substituents each independently can be amino, halo, hydroxy, alkoxy, sulfonamide, haloalkyl, cyano, nitro, an optionally substituted cycloaliphatic, or an optionally substituted heterocycloaliphatic.

In some embodiments, R³ is a cycloaliphatic or a heterocycloaliphatic, each of which is optionally substituted with halo, amino, hydroxy, oxo, alkoxy, alkyl, sulfonamide. The alkyl substitutent or the alkyl moiety in the alkoxy substituent can contain 1 to 12 (e.g., 1 to 4 or 1 to 6) carbon atoms.

In some embodiments, n is 0.

In some embodiments X is S, O, or N(R). In still some further embodiments, X is O or N(R).

In some embodiments R³X— is

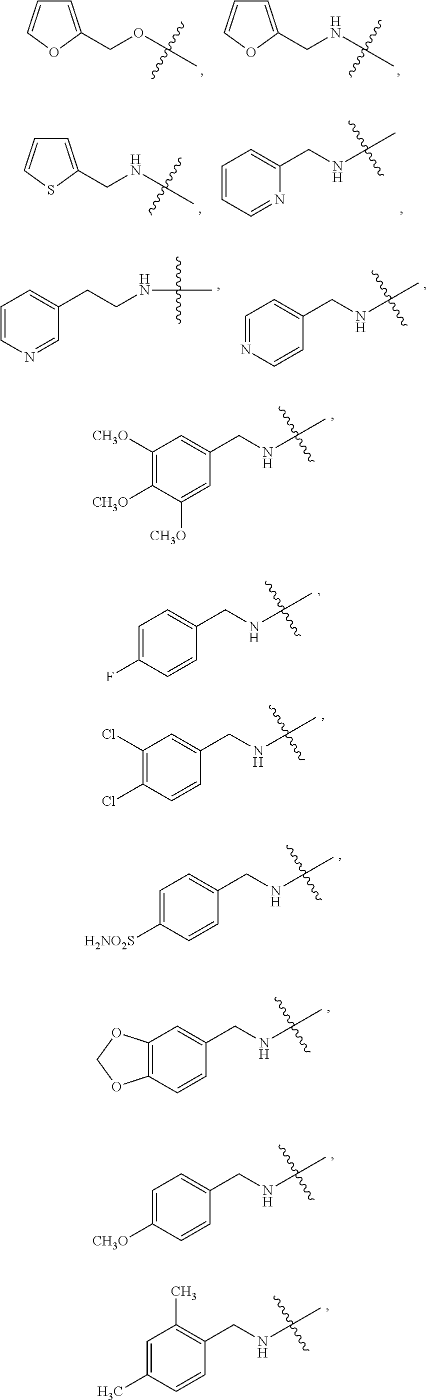

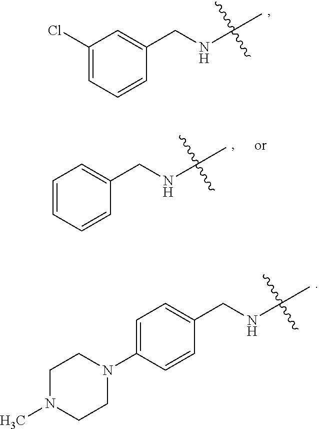

In other embodiments, R³X is:

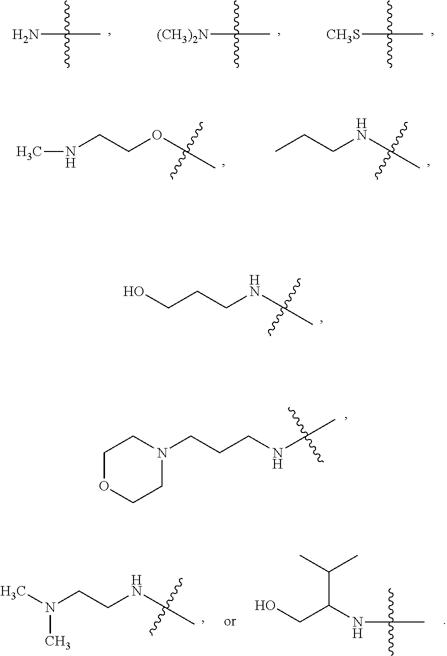

In some embodiments, R³X— is

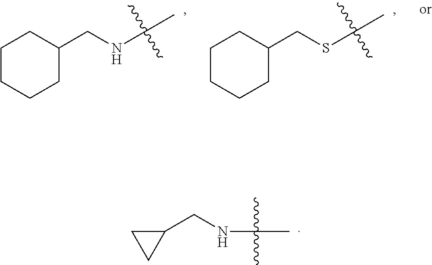

In some further embodiments, R³X— is

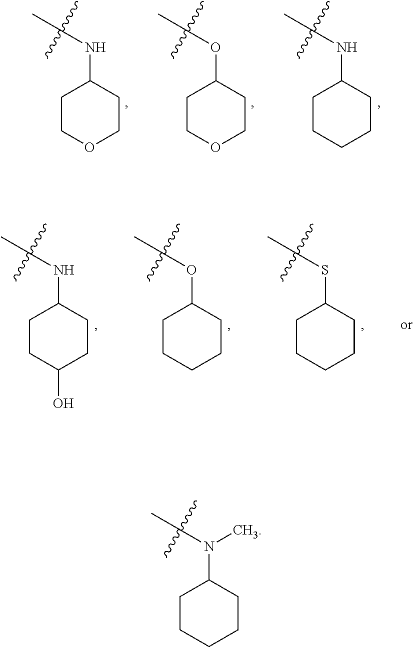

In some embodiments, X is N(R); and R and R³, together with the nitrogen atom to which they are attached, form an optionally substituted heterocycloaliphatic or heteroaryl ring.

In some further embodiments, R³X— is

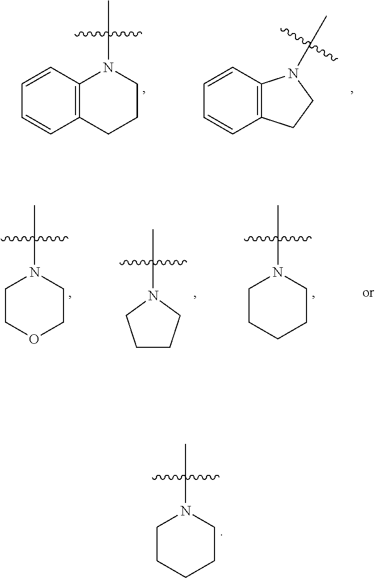

In some embodiments, R³ is an optionally substituted aryl.

In some further embodiments, R² is phenyl or napthyl, both of which are substituted with 1 to 3 substituents independently selected from the group consisting of halo, cyano, nitro, hydroxy, alkoxy, alkoxy-alkoxy, haloalkoxy, haloalkyl, alkylsulfanyl, alkyl, alkenyl, alkynyl, silylalkenyl, alkylcarbonylalkyl, and carboxy. The alkyl substituent or the alkyl moiety in these optional substituents can contain 1 to 12 (e.g., 1 to 6 or 1 to 4) carbon atoms.

In some further embodiments, R³ is phenyl optionally substituted with cyano, halo, haloalkyl, amino, hydroxy, alkoxy, carboxy (e.g., alkoxycarbonyl), amido, alkyl, alkylcarbonylalkyl, sulfonamide, cycloaliphatic, or heterocycloaliphatic. The number of these optional substituents can be 1, 2, 3, or 4.

In some further embodiments, R³X— is

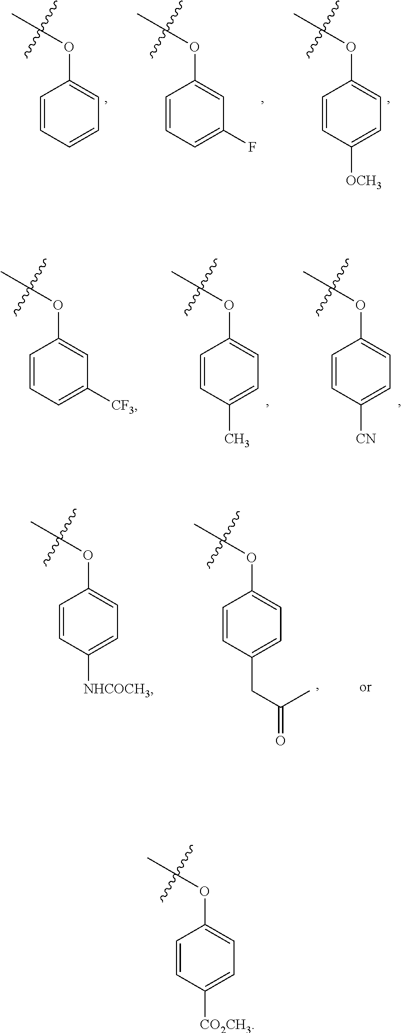

In still some further embodiments, R³X— is

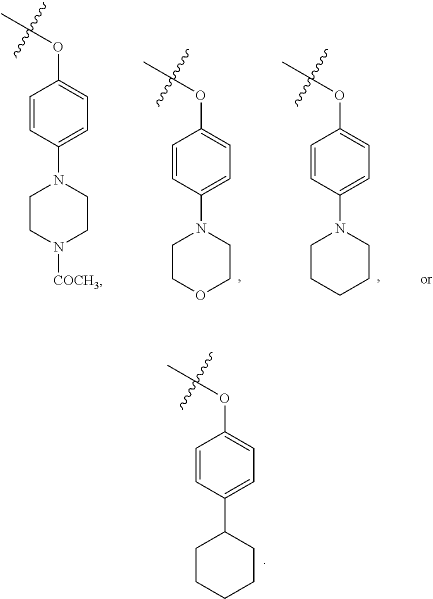

In some embodiments, R² is H, halo, or an amino (e.g., alkylamino or arylamino).

In some other embodiments, R² is

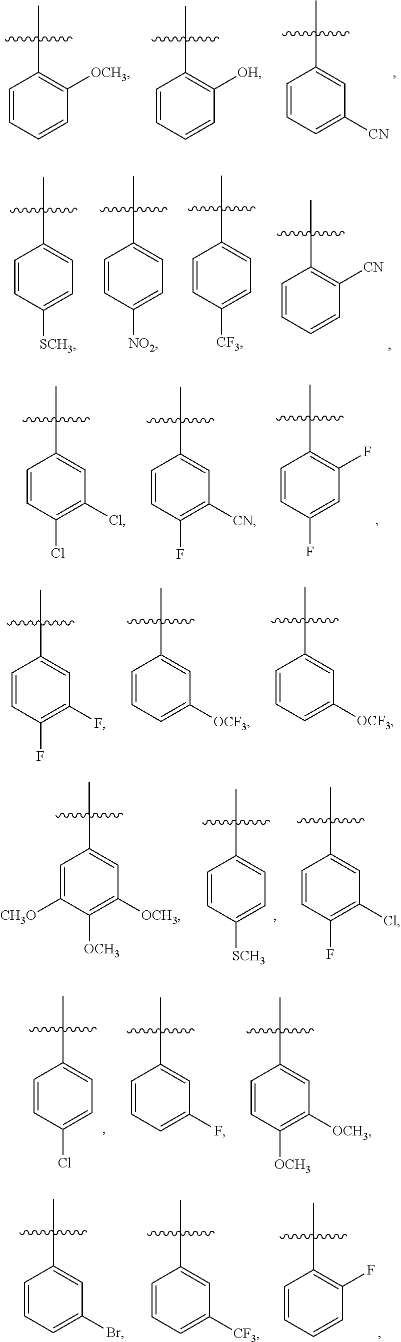

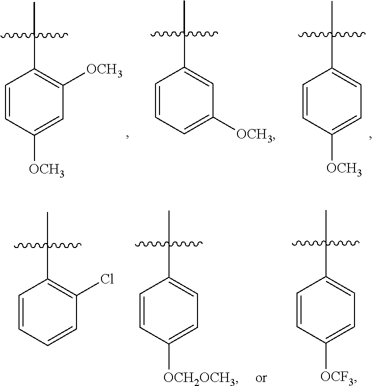

In some other embodiments, R² is

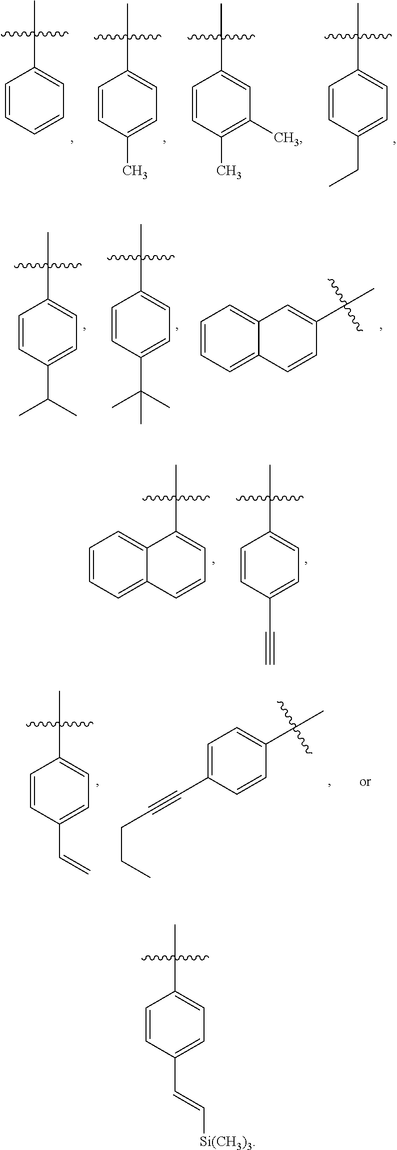

In some other embodiments, R² is

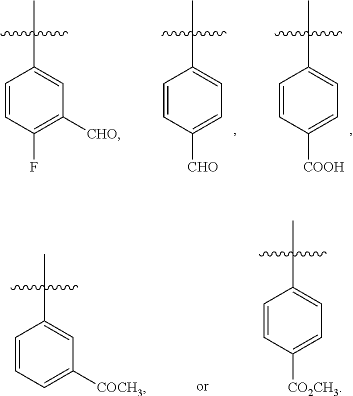

In some embodiments, R² is an optionally substituted heteroaryl.

In some further embodiments, R² is pyrimidinyl, pyridinyl, indolyl, thiophenyl, quinoxalinyl, benzo-oxadiazole, pyrrolyl, triazolyl, tetrazolyl, indazolyl, benzofuranyl, dihydrobenzo-oxazine, furanyl, benzothiophenyl, quinolinyl, or pyrazolyl; each of which is optionally substituted with halo, cyano, alkyl, aralkyl, alkoxy, carboxy (e.g., alkoxycarbonyl or hydroxycarbonyl), acyl (e.g., alkylcarbonyl or hydro carbonyl), hydroxyalkyl, or alkoxyalkyl.

In still some further embodiments, R² is

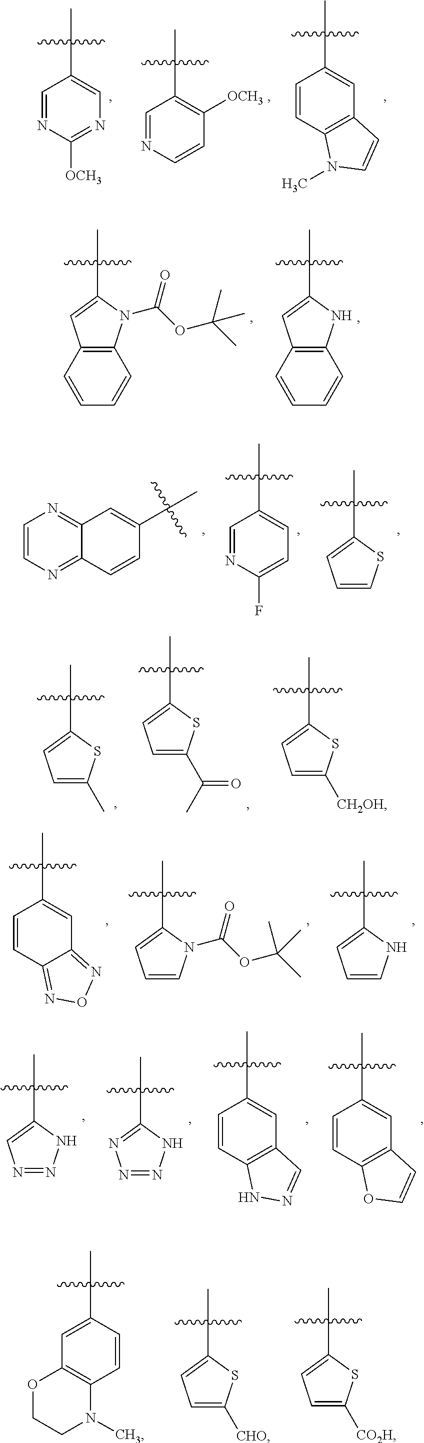

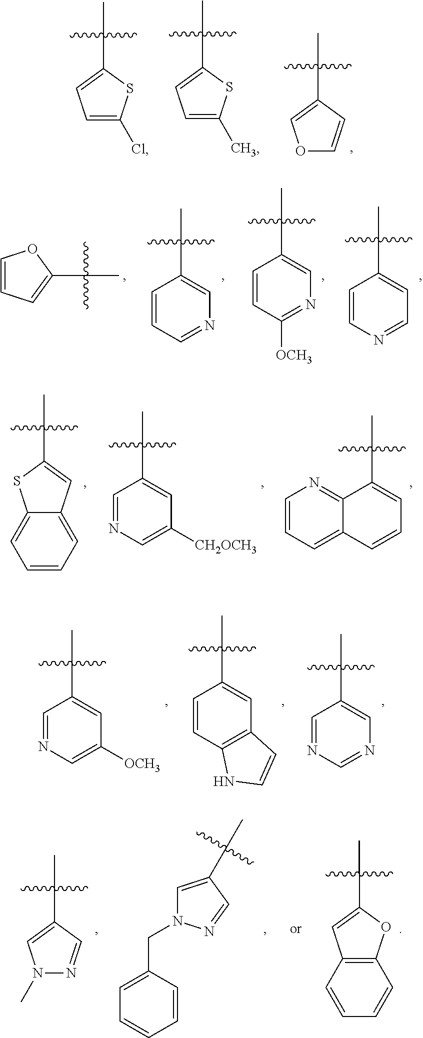

In some embodiments, R² is an optionally substituted alkyl (e.g., (arylcarbonyl)alkyl), an optionally substituted alkenyl, an optionally substituted alkynyl (e.g., arylpropynyl such as phenylpropynyl), an optionally substituted heterocycloalkenyl, or an optionally substituted cycloalkenyl.

In some further embodiments, R² is

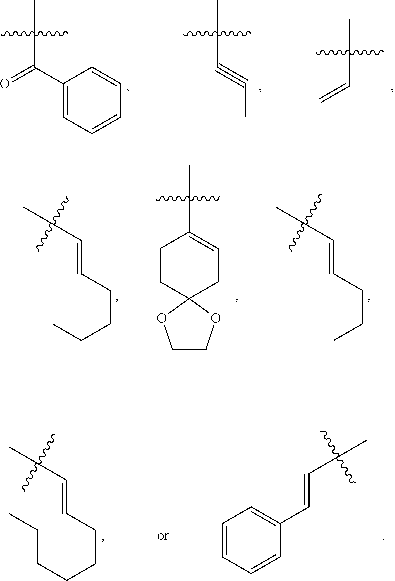

In some embodiments, the compound of Formula (I) is
N-(2-hydroxyethyl)-3-(6-(thiophen-2-ylmethylamino)imidazo[1,2-b]pyridazin-3-yl)benzamide;
N-(furan-2-ylmethyl)-3-(5-isopropyl-2-methoxyphenyl)imidazo[1,2-b]pyridazin-6-amine;
3-(6-(furan-2-ylmethylamino)imidazo[1,2-b]pyridazin-3-yl)benzoic acid;
N-(pyridin-2-ylmethyl)-3-(4-(trifluoromethoxy)phenyl)imidazo[1,2-b]pyridazin-6-amine;
3-(thiophen-2-yl)-N-(thiophen-2-ylmethyl)imidazo[1,2-b]pyridazin-6-amine;
N-(3-(6-(thiophen-2-ylmethylamino)imidazo[1,2-b]pyridazin-3-yl)phenyl)acetamide;
3-(4-aminophenyl)-N-(thiophen-2-ylmethyl)imidazo[1,2-b]pyridazin-6-amine;
4-(6-(furan-2-ylmethylamino)imidazo[1,2-b]pyridazin-3-yl)phenol;
2-methoxy-4-(6-(thiophen-2-ylmethylamino)imidazo[1,2-b]pyridazin-3-yl)phenol;
2-(3-(5-isopropyl-2-methoxyphenyl)imidazo[1,2-b]pyridazin-6-ylamino)-3-methylbutan-1-ol;
N-(2-methoxyethyl)-3-(naphthalen-2-yl)imidazo[1,2-b]pyridazin-6-amine;
3-(3-aminophenyl)-N-(tetrahydro-2H-pyran-4-yl)imidazo[1,2-b]pyridazin-6-amine;
3-(benzo[d][1;3]dioxol-5-yl)-N-(pyridin-2-ylmethyl)imidazo[1,2-b]pyridazin-6-amine;
N-(2-methoxyethyl)-3-(quinolin-8-yl)imidazo[1,2-b]pyridazin-6-amine;
N-(3-(6-(2-methoxyethylamino)imidazo[1,2-b]pyridazin-3-yl)phenyl)acetamide;
N-(3-(6-(tetrahydro-2H-pyran-4-ylamino)imidazo[1,2-b]pyridazin-3-yl)phenyl)acetamide;
4-((3-(3,4-dimethoxyphenyl)imidazo[1,2-b]pyridazin-6-ylamino)methyl)-benzenesulfonamide;
4-((3-(4-(trifluoromethoxy)phenyl)imidazo[1,2-b]pyridazin-6-ylamino)methyl)benzene-sulfonamide;
N-(tetrahydro-2H-pyran-4-yl)-3-(3,4,5-trimethoxyphenyl)imidazo[1,2-b]pyridazin-6-amine;
3-(3-(dimethylamino)phenyl)-N-(tetrahydro-2H-pyran-4-yl)imidazo[1,2-b]pyridazin-6-amine;
(E)-3-(3-(6-(3-hydroxypropylamino)imidazo[1,2-b]pyridazin-3-yl)phenyl)acrylic acid;
3-(3-(1-benzyl-1H-pyrazol-4-yl)imidazo[1,2-b]pyridazin-6-ylamino)propan-1-ol;
N-(tetrahydro-2H-pyran-4-yl)-3-(3-(trifluoromethoxy)phenyl)imidazo[1,2-b]pyridazin-6-amine;
4-((3-(4-(hydroxymethyl)phenyl)imidazo[1,2-b]pyridazin-6-ylamino)methyl)benzenesulfonamide;
4-(6-(3-hydroxypropylamino)imidazo[1,2-b]pyridazin-3-yl)-2-methoxyphenol;
(E)-3-(3-(6-(2-methoxyethylamino)imidazo[1,2-b]pyridazin-3-yl)phenyl)acrylic acid;
4-((3-(6-methoxypyridin-3-yl)imidazo[1,2-b]pyridazin-6-ylamino)methyl)benzenesulfonamide;
3-(6-(3,4,5-trim ethoxybenzyl amino)imidazo[1,2-b]pyridazin-3-yl)phenol;
4-((3-(4-hydroxy-3-methoxyphenyl)imidazo[1,2-b]pyridazin-6-ylamino)methyl)benzene-sulfonamide;
3-(5-methoxypyridin-3-yl)-N-(tetrahydro-2H-pyran-4-yl)imidazo[1,2-b]pyridazin-6-amine;
3-(6-(2-methoxyethylamino)imidazo[1,2-b]pyridazin-3-yl)benzaldehyde;
(E)-3-(3-(hex-1-enyl)phenyl)-N-(tetrahydro-2H-pyran-4-yl)imidazo[1,2-b]pyridazin-6-amine;
4-((3-(3-formylphenyl)imidazo[1,2-b]pyridazin-6-ylamino)methyl)benzenesulfonamide;
3-(5-isopropyl-2-methoxyphenyl)-N-(tetrahydro-2H-pyran-4-yl)imidazo[1,2-b]pyridazin-6-amine;
1-(3-(6-(2-methoxyethylamino)imidazo[1,2-b]pyridazin-3-yl)phenyl)ethanone;
N-(2-methoxyethyl)-3-(4-morpholinophenyl)imidazo[1,2-b]pyridazin-6-amine;
N-(benzo[d][1,3]dioxol-5-ylmethyl)-3-(pyridin-4-yl)imidazo[1,2-b]pyridazin-6-amine;
3-(4-(dimethylamino)phenyl)-N-(tetrahydro-2H-pyran-4-yl)imidazo[1,2-b]pyridazin-6-amine;
3-phenyl-N-(tetrahydro-2H-pyran-4-yl)imidazo[1,2-b]pyridazin-6-amine;
3-(naphthalen-1-yl)-N-(tetrahydro-2H-pyran-4-yl)imidazo[1,2-b]pyridazin-6-amine;
3-(2-phenoxyphenyl)-N-(tetrahydro-2H-pyran-4-yl)imidazo[1,2-b]pyridazin-6-amine;
3-(benzo[d][1,3]dioxol-5-yl)-N-(tetrahydro-2H-pyran-4-yl)imidazo[1,2-b]pyridazin-6-amine;
3-(3-(3-aminophenyl)imidazo[1,2-b]pyridazin-6-ylamino)propan-1-ol;
3-(3-(benzo[d][1,3]dioxol-5-yl)imidazo[1,2-b]pyridazin-6-ylamino)propan-1-ol;
4-(6-(3-hydroxypropylamino)imidazo[1,2-b]pyridazin-3-yl)phenol;
N-(benzo[d][1,3]dioxol-5-ylmethyl)-3-(pyridin-3-yl)imidazo[1,2-b]pyridazin-6-amine;
3-(pyridin-3-yl)-N-(tetrahydro-2H-pyran-4-yl)imidazo[1,2-b]pyridazin-6-amine;
(E)-3-styryl-N-(tetrahydro-2H-pyran-4-yl)imidazo[1,2-b]pyridazin-6-amine;
3-(3,4-dimethoxyphenyl)-N-(tetrahydro-2H-pyran-4-yl)imidazo[1,2-b]pyridazin-6-amine;
4-(6-(tetrahydro-2H-pyran-4-ylamino)imidazo[1,2-b]pyridazin-3-yl)phenol;
3-(4-(6-(tetrahydro-2H-pyran-4-ylamino)imidazo[1,2-b]pyridazin-3-yl)phenyl)propanoic acid;
3-(2-methoxyphenyl)-N-(tetrahydro-2H-pyran-4-yl)imidazo[1,2-b]pyridazin-6-amine;
4-(6-(4-methoxybenzylamino)imidazo[1,2-b]pyridazin-3-yl)phenol;
3-(3-(3,4-dimethoxyphenyl)imidazo[1,2-b]pyridazin-6-ylamino)propan-1-ol;
4-((3-(4-hydroxyphenyl)imidazo[1,2-b]pyridazin-6-ylamino)methyl)benzenesulfonamide;
4-(6-(tetrahydro-2H-pyran-4-ylamino)imidazo[1,2-b]pyridazin-3-yl)benzonitrile;
4-(6-(tetrahydro-2H-pyran-4-ylamino)imidazo[1,2-b]pyridazin-3-yl)benzoic acid;
3-(furan-2-yl)-N-(tetrahydro-2H-pyran-4-yl)imidazo[1,2-b]pyridazin-6-amine;
3-(4-chlorophenyl)-N-(tetrahydro-2H-pyran-4-yl)imidazo[1,2-b]pyridazin-6-amine;
3-(3-chloro-4-fluorophenyl)-N-(tetrahydro-2H-pyran-4-yl)imidazo[1,2-b]pyridazin-6-amine;
3-(3,4-dimethylphenyl)-N-(tetrahydro-2H-pyran-4-yl)imidazo[1,2-b]pyridazin-6-amine;
3-(3-(3-(dimethyl amino)phenyl)imidazo[1,2-b]pyridazin-6-ylamino)propan-1-ol;
(E)-3-(3-(hex-1-enyl)phenyl)-N-(3-methoxypropyl)imidazo[1,2-b]pyridazin-6-amine;
N-(benzo[d][1,3]dioxol-5-ylmethyl)-3-(5-methoxypyridin-3-yl)imidazo[1,2-b]pyridazin-6-amine;
N-(4-methoxyphenyl)-4-(6-(tetrahydro-2H-pyran-4-ylamino)imidazo[1,2-b]pyridazin-3-yl)benzamide;
3-(1H-indol-5-yl)-N-(tetrahydro-2H-pyran-4-yl)imidazo[1,2-b]pyridazin-6-amine;
3-(3-bromophenyl)-N-(tetrahydro-2H-pyran-4-yl)imidazo[1,2-b]pyridazin-6-amine;
3-(3-chlorophenyl)-N-(tetrahydro-2H-pyran-4-yl)imidazo[1,2-b]pyridazin-6-amine;
3-(4-methoxyphenyl)-N-(tetrahydro-2H-pyran-4-yl)imidazo[1,2-b]pyridazin-6-amine;
3-(6-(tetrahydro-2H-pyran-4-ylamino)imidazo[1,2-b]pyridazin-3-yl)phenol;
2-methoxy-4-(6-(2-methoxyethylamino)imidazo[1,2-b]pyridazin-3-yl)phenol;
2-methoxy-4-(6-(3,4,5-trimethoxybenzylamino)imidazo[1,2-b]pyridazin-3-yl)phenol;
N-(benzo[d][1,3]dioxol-5-ylmethyl)-3-(6-methoxypyridin-3-yl)imidazo[1,2-b]pyridazin-6-amine;
4-(6-(benzo[d][1,3]dioxol-5-ylmethylamino)imidazo[1,2-b]pyridazin-3-yl)-2-methoxyphenol;
(2-(6-(tetrahydro-2H-pyran-4-ylamino)imidazo[1,2-b]pyridazin-3-yl)phenyl)methanol;
3-(6-methoxypyridin-3-yl)-N-(tetrahydro-2H-pyran-4-yl)imidazo[1,2-b]pyridazin-6-amine;
N-(tetrahydro-2H-pyran-4-yl)-3-(3-(trifluoromethyl)phenyl)imidazo[1,2-b]pyridazin-6-amine;
3-(3-fluorophenyl)-N-(tetrahydro-2H-pyran-4-yl)imidazo[1,2-b]pyridazin-6-amine;
3-(4-amino phenyl)-N-(tetrahydro-2H-pyran-4-yl)imidazo[1,2-b]pyridazin-6-amine;
3-(4-fluorophenyl)-N-(tetrahydro-2H-pyran-4-yl)imidazo[1,2-b]pyridazin-6-amine;
3-(furan-3-yl)-N-(tetrahydro-2H-pyran-4-yl)imidazo[1,2-b]pyridazin-6-amine;
2-methoxy-4-(6-(4-methoxybenzylamino)imidazo[1,2-b]pyridazin-3-yl)phenol;
3-(4-aminophenyl)-N-(4-methoxybenzyl)imidazo[1,2-b]pyridazin-6-amine;
3-(4-phenoxyphenyl)-N-(tetrahydro-2H-pyran-4-yl)imidazo[1,2-b]pyridazin-6-amine;
3-(pyrimidin-5-yl)-N-(tetrahydro-2H-pyran-4-yl)imidazo[1,2-b]pyridazin-6-amine;
methyl 4-(6-(tetrahydro-2H-pyran-4-ylamino)imidazo[1,2-b]pyridazin-3-yl)benzoate;
3-(2-chlorophenyl)-N-(tetrahydro-2H-pyran-4-yl)imidazo[1,2-b]pyridazin-6-amine;
3-(2-fluorophenyl)-N-(tetrahydro-2H-pyran-4-yl)imidazo[1,2-b]pyridazin-6-amine;
N-(tetrahydro-2H-pyran-4-yl)-3-p-tolylimidazo[1,2-b]pyridazin-6-amine;
3-(4-methoxyphenyl)-N-(tetrahydro-2H-pyran-4-yl)imidazo[1,2-b]pyridazin-6-amine;
3-(3,5-dimethoxyphenyl)-N-(tetrahydro-2H-pyran-4-yl)imidazo[1,2-b]pyridazin-6-amine;
1-(3-(6-(tetrahydro-2H-pyran-4-ylamino)imidazo[1,2-b]pyridazin-3-yl)phenyl)ethanone;
N-(tetrahydro-2H-pyran-4-yl)-3-(thiophen-2-yl)imidazo[1,2-b]pyridazin-6-amine;
4-((3-(1-methyl-1H-pyrazol-4-yl)imidazo[1,2-b]pyridazin-6-ylamino)methyl)benzenesulfonamide;
4-((3-(1-benzyl-1H-pyrazol-4-yl) imidazo[1,2-b]pyridazin-6-ylamino)methyl)benzenesulfonamide;
3-(naphthalen-2-yl)-N-(2-(pyridin-3-yl)ethyl)imidazo[1,2-b]pyridazin-6-amine;
3-(naphthalen-2-yl)-N-(pyridin-4-ylmethyl)imidazo[1,2-b]pyridazin-6-amine;
3-(3,4-dimethoxyphenyl)-N-(furan-2-yl methyl)imidazo[1,2-b]pyridazin-6-amine;
N-(furan-2-ylmethyl)-3-(4-(trifluoromethoxy)phenyl)imidazo[1,2-b]pyridazin-6-amine;
4-(6-(thiophen-2-ylmethylamino)imidazo[1,2-b]pyridazin-3-yl)phenol;
(R)-N-(3-(6-(1-hydroxy-3-methylbutan-2-ylamino)imidazo[1,2-b]pyridazin-3-yl)-phenyl)acetamide;
N-(furan-2-ylmethyl)-3-(4-methoxyphenyl)imidazo[1,2-b]pyridazin-6-amine;
(4-(6-(furan-2-ylmethylamino)imidazo[1,2-b]pyridazin-3-yl)phenyl)methanol;
4-(6-(cyclopropylmethylamino)imidazo[1,2-b]pyridazin-3-yl)-2-methoxyphenol;
4-(6-(furan-2-ylmethylamino)imidazo[1,2-b]pyridazin-3-yl)-2-methoxyphenol;
(R)-4-(6-(1-hydroxy-3-methylbutan-2-ylamino)imidazo[1,2-b]pyridazin-3-yl)-2-methoxyphenol;
N-(furan-2-ylmethyl)-3-(4-phenoxyphenyl)imidazo[1,2-b]pyridazin-6-amine;
3-(benzofuran-2-yl)-N-(3-chlorobenzyl)imidazo[1,2-b]pyridazin-6-amine;
4-(6-(3-chlorobenzylamino)imidazo[1,2-b]pyridazin-3-yl)phenol;
4-(6-(4-fluorobenzylamino)imidazo[1,2-b]pyridazin-3-yl)phenol;
N-(4-(4-methylpiperazin-1-yl)benzyl)-3-(4-(trifluoromethoxy)phenyl)imidazo[1,2-b]pyridazin-6-amine;
3-(6-(4-(4-methylpiperazin-1-yl)benzylamino)imidazo[1,2-b]pyridazin-3-yl)phenol;
4-(6-(3-chlorobenzylamino)imidazo[1,2-b]pyridazin-3-yl)-2-methoxyphenol;
2-methoxy-4-(6-(propylamino)imidazo[1,2-b]pyridazin-3-yl)phenol;
4-(6-(3,4-dichlorobenzylamino)imidazo[1,2-b]pyridazin-3-yl)-2-methoxyphenol;
4-(6-(2,4-dimethylbenzylamino)imidazo[1,2-b]pyridazin-3-yl)-2-methoxyphenol;
4-(6-(3-chlorobenzylamino)imidazo[1,2-b]pyridazin-3-yl)-N-(2-(dimethylamino)ethyl)benzamide;
N-(3-morpholinopropyl)-3-(naphthalen-2-yl)imidazo[1,2-b]pyridazin-6-amine;
N¹,N¹-dimethyl-N³-(3-(naphthalen-2-yl)imidazo[1,2-b]pyridazin-6-yl)propane-1;3-diamine; or
N-(3-(6-(furan-2-ylmethylamino)imidazo[1,2-b]pyridazin-3-yl)phenyl)methanesulfonamide.

The compounds of Formula (I) generally described above or the specific compounds specifically listed above are also within the scope of this invention.

In another aspect, the invention also relates to a method of treating an IRAK-responsive condition or disorder in a subject. This method includes administering to the subject in need of such a treatment a therapeutically effective amount of one of the compounds described or listed above.

In some embodiments, the condition or disorder is rheumatoid arthritis, multiple sclerosis, sepsis, osteoarthritis, inflammatory bowel disease, osteoporosis, myasthenia gravis, stroke, Alzheimer's disease, Parkinson's disease, amyotrophic lateral sclerosis, psoriasis, cardiac contractile dysfunction, type I diabetes, type II diabetes, familial cold autoinflammatory syndrome, severe bacterial infections, allergic disease, cancer, psoriasis, asthma, or graft rejection.

In some embodiments, the compound is administered orally, parenterally, or topically.

The invention further relates to a method of treating a condition or disorder mediated by IRAK or by NF-κB in a subject, which includes administering to the subject in need of such a treatment a therapeutically effective amount of any of the compounds described above. Similarly, the compound can be administered orally, parenterally, or topically.

The invention is also directed to a method for modulating an IRAK kinase, which includes contacting the IRAK kinase or a cell with one of the compounds described or listed above.

In some embodiments, the compound inhibits the IRAK kinase. In some other embodiments, the compound activates the IRAK kinase.

The invention is further directed to a method for decreasing NF-κB activation, which includes contacting a cell with one of the compounds described above.

In some other embodiments, the compound is administered in combination with a second therapeutic agent. Examples of such a second therapeutic agent include methotrexate, sulfasalazine, a COX-2 inhibitor, hydroxychloroquine, cyclosporine A, D-penicillamine, infliximab, etanercept, auranofin, aurothioglucose, sulfasalazine, sulfasalazine analogs, mesalamine, corticosteroids, corticosteroid analogs, 6-mercaptopurine, cyclosporine A, methotrextate and infliximab, interferon beta-1 beta, interferon beta-1 alpha, azathioprine, glatiramer acetate, a glucocorticoid, or cyclophosphamide.

The invention further provides pharmaceutical compositions each containing a compound of Formula (I) as described above or a compound specifically identified above, and methods of using a compound of Formula (I) for modulating the function of IRAK kinase for the treatment of inflammatory, cell proliferative and immune-related conditions or diseases associated with IRAK-mediated signal transduction, such as rheumatoid arthritis, inflammatory bowel disease, multiple sclerosis, diabetes, obesity, allergic disease, psoriasis, asthma, graft rejection, cancer, and sepsis.

For purposes of this invention, the chemical elements are identified in accordance with the Periodic Table of the Elements, CAS version, Handbook of Chemistry and Physics, 75^{th} Ed. Additionally, general principles of organic chemistry are described by Thomas Sorrell in Organic Chemistry, University Science Books, Sausalito (1999); and by M. B. Smith and J. March in Advanced Organic Chemistry, 5^{th} Ed., John Wiley & Sons, New York (2001), the entire contents of which are hereby incorporated by reference.

The term “modulating” as used herein means increasing or decreasing, e.g. activity, by a measurable amount. Compounds that modulate the function of IRAK proteins by increasing their activity are called agonists. Compounds that modulate the function of IRAK proteins by decreasing their activity are called antagonists.

The phrase “treating or reducing the severity of an IRAK mediated disease” refers both to treatments for diseases that are directly caused by IRAK activities and alleviation of symptoms of diseases not directly caused by IRAK activities.

As described herein, compounds of the invention may optionally be substituted with one or more substituents, such as those as generally illustrated above, or as specifically exemplified by particular classes, subclasses, and species of the invention.

As used herein, the term “aliphatic” encompasses alkyl, alkenyl, and alkynyl, each of which is optionally substituted as set forth below. Unless otherwise specified, it encompasses both a branched group (e.g., tert-alkyl such as tert-butyl) and a straight aliphatic chain (e.g., n-alkyl groups, alkenyl groups, or alkynyl groups). A straight aliphatic chain has the structure of —(CH₂)ᵥ-, wherein v can be any integer, e.g., from 1 to 12 (such as 1 to 4 or 1 to 6). A branched aliphatic chain is a straight aliphatic chain that is substituted with one or more aliphatic groups. A branched aliphatic chain has the structure —[CQQ′]ᵥ- wherein at least one of Q and Q′ is an aliphatic group and v can be any integer, e.g., from 1 to 12 (such as 1 to 4 or 1 to 6).

As used herein, an “alkyl” group refers to a saturated aliphatic hydrocarbon group containing 1 to 8 (e.g., 1 to 4 or 1 to 6) carbon atoms. An alkyl group can be straight or branched. Examples of alkyl groups include, but are not limited to, methyl, ethyl, propyl, isopropyl, butyl, isobutyl, sec-butyl, tert-butyl, n-pentyl, n-heptyl, and 2-ethylhexyl. An alkyl group can be substituted (i.e., optionally substituted) with one or more substituents such as halo; cycloaliphatic (e.g., cycloalkyl or cycloalkenyl); heterocycloaliphatic (e.g., heterocycloalkyl or heterocycloalkenyl); aryl; heteroaryl; alkoxy; aroyl; heteroaroyl; acyl (e.g., (aliphatic)carbonyl, (cycloaliphatic)carbonyl, or (heterocycloaliphatic)carbonyl); nitro; cyano; amido (e.g., (cycloalkylalkyl)amido, arylamidoo, aralkylamido, (heterocycloalkyl)amido, (heterocycloalkylalkyl)amido, heteroarylamido, heteroaralkylamido alkylamido, cycloalkylamido, heterocycloalkylamido, arylamido, or heteroarylamido); amino (e.g., aliphaticamino, cycloaliphaticamino, or heterocycloaliphaticamino); oxime; sulfonyl (e.g., aliphatic-S(O)₂—); sulfinyl; sulfanyl; sulfoxy; urea; thiourea; sulfonamide; sulfamide; oxo (thus forming a carbonyl group, i.e., —CO—); carboxy; carbamoyl; cycloaliphaticoxy; heterocycloaliphaticoxy; aryloxy; heteroaryloxy; aralkyloxy; heteroarylalkoxy; alkoxycarbonyl; alkylcarbonyloxy; or hydroxy. Without limitation, examples of substituted alkyls include carboxyalkyl (such as HOOC-alkyl, alkoxycarbonylalkyl, and alkylcarbonyloxyalkyl); cyanoalkyl; hydroxyalkyl; alkoxyalkyl; acylalkyl; aralkyl; (alkoxyaryl)alkyl; (sulfonylamino)alkyl (e.g., alkyl-S(O)₂-aminoalkyl); aminoalkyl; amidoalkyl; (cycloaliphatic)alkyl; silyl (e.g. trialkylsilyl); and haloalkyl.

As used herein, an “alkenyl” group refers to an aliphatic carbon group that contains 2 to 8 (e.g., 2 to 4 or 2 to 6) carbon atoms and at least one double bond. Like an alkyl group, an alkenyl group can be straight or branched. Examples of an alkenyl group include, but are not limited to, allyl, isoprenyl, 2-butenyl, and 2-hexenyl. An alkenyl group can be optionally substituted with one or more substituents, such as halo; cycloaliphatic (e.g., cycloalkyl or cycloalkenyl); heterocycloaliphatic (e.g., heterocycloalkyl or heterocycloalkenyl); aryl; heteroaryl; alkoxy; aroyl; heteroaroyl; acyl (e.g., (aliphatic)carbonyl, (cycloaliphatic)carbonyl, or (heterocycloaliphatic)carbonyl); nitro; cyano; amido (e.g., (cycloalkylalkyl)amido, arylamido, aralkylamido, (heterocycloalkyl)amido, (heterocycloalkylalkyl)amido, heteroarylamido, heteroaralkylamido alkylaminocarbonyl, cycloalkylaminocarbonyl, heterocycloalkylaminocarbonyl, arylaminocarbonyl, or heteroarylaminocarbonyl); amino (e.g., aliphaticamino, cycloaliphaticamino, heterocycloaliphaticamino, or aliphaticsulfonylamino); oxime; sulfonyl (e.g., alkyl-S(O)₂—, cycloaliphatic-S(O)₂—, or aryl-S(O)₂—); sulfinyl; sulfanyl; sulfoxy; urea; thiourea; sulfonamide; sulfamide; oxo; carboxy; carbamoyl; cycloaliphaticoxy; heterocycloaliphaticoxy; aryloxy; heteroaryloxy; aralkyloxy; heteroaralkoxy; alkoxycarbonyl; alkylcarbonyloxy; or hydroxy. Without limitation, some examples of substituted alkenyls include cyanoalkenyl, alkoxyalkenyl, acylalkenyl, hydroxyalkenyl, aralkenyl, (alkoxyaryl)alkenyl, (sulfonylamino)alkenyl (such as (alkyl-S(O)₂-aminoalkenyl), aminoalkenyl, amidoalkenyl, (cycloaliphatic)alkenyl, and haloalkenyl.

As used herein, an “alkynyl” group refers to an aliphatic carbon group that contains 2 to 8 (e.g., 2 to 6 or 2 to 4) carbon atoms and has at least one triple bond. An alkynyl group can be straight or branched. Examples of an alkynyl group include, but are not limited to, propargyl and butynyl. An alkynyl group can be optionally substituted with one or more substituents such as aroyl; heteroaroyl; alkoxy; cycloalkyloxy; heterocycloalkyloxy; aryloxy; heteroaryloxy; aralkyloxy; nitro; carboxy; cyano; halo; hydroxy; sulfo; mercapto; sulfanyl (e.g., aliphatic-S— or cycloaliphatic-S—); sulfinyl (e.g., aliphatic-S(O)— or cycloaliphatic-S(O)—); sulfonyl (e.g., aliphatic-S(O)₂—, aliphaticamino-S(O)₂—, or cycloaliphatic-S(O)₂—); amido (e.g., alkylamido, alkylamido, cycloalkylamido, heterocycloalkylamido, cycloalkylamido, arylamido, arylamido, aralkylamido, (heterocycloalkyl)amido, (cycloalkylalkyl)amido, heteroaralkylamido, heteroarylamido or heteroarylamido); urea; thiourea; sulfonamide; sulfamide; alkoxycarbonyl; alkylcarbonyloxy; cycloaliphatic; heterocycloaliphatic; aryl; heteroaryl; acyl (e.g., (cycloaliphatic)carbonyl or (heterocycloaliphatic)carbonyl); amino (e.g., aliphaticamino); sulfoxy; oxo; carbamoyl; (cycloaliphatic)oxy; (heterocycloaliphatic)oxy; or (heteroaryl)alkoxy.

As used herein, the term “amido” encompasses both “aminocarbonyl” and “carbonylamino.” Each of these terms, when used alone or in connection with another group, refers to an amido group such as —N(R^{X})—C(O)—R^{Y} or —C(O)—N(R^{X})₂, when used terminally; or —C(O)—N(R^{X})— or —N(R^{X})—C(O)— when used internally, wherein R^{X} and R^{Y} are defined below. Examples of amido groups include alkylamido (such as alkylcarbonylamino or alkylaminocarbonyl), (heterocycloaliphatic)amido, (heteroaralkyl)amido, (heteroaryl)amido, (heterocycloalkyl)alkylamido, arylamido, aralkylamido, (cycloalkyl)alkylamido, and cycloalkylamido.

As used herein, an “amino” group refers to —N(R^{X})(R^{Y}) wherein each of R^{X} and R^{Y} is independently hydrogen (or sometimes “H” hereinafter), alkyl, cycloaliphatic, (cycloaliphatic)aliphatic, aryl, araliphatic, heterocycloaliphatic, (heterocycloaliphatic)aliphatic, heteroaryl, carboxy, sulfanyl, sulfinyl, sulfonyl, (aliphatic)carbonyl, (cycloaliphatic)carbonyl, ((cycloaliphatic)aliphatic)carbonyl, arylcarbonyl, (araliphatic)carbonyl, (heterocycloaliphatic)carbonyl, ((heterocycloaliphatic)aliphatic)carbonyl, (heteroaryl)carbonyl, or (heteroaraliphatic)carbonyl, each of which being defined herein and being optionally substituted. Examples of amino groups include alkylamino, dialkylamino, aryl amino, and diarylamino. When the term “amino” is not the terminal group (e.g., alkylcarbonylamino), it is represented by —N(R^{X})—. R^{X} has the same meaning as defined above.

As used herein, an “aryl” group, used alone or as part of a larger moiety such as in “aralkyl”, “aralkoxy,” or “aryloxyalkyl,” refers to monocyclic (e.g., phenyl); bicyclic (e.g., indenyl, naphthalenyl, tetrahydronaphthyl, or tetrahydroindenyl); and tricyclic (e.g., fluorenyl tetrahydrofluorenyl, tetrahydroanthracenyl, or anthracenyl) ring systems in which the monocyclic ring system is aromatic or at least one of the rings in a bicyclic or tricyclic ring system is aromatic. The bicyclic and tricyclic groups include benzofused 2- or 3-membered carbocyclic rings. For instance, a benzofused group includes phenyl fused with two or more C₄₋₈ carbocyclic moieties. An aryl is optionally substituted with one or more substituents including aliphatic (e.g., alkyl, alkenyl, or alkynyl); cycloaliphatic; (cycloaliphatic)aliphatic; heterocycloaliphatic; (heterocycloaliphatic)aliphatic; aryl; heteroaryl; alkoxy; (cycloaliphatic)oxy; (heterocycloaliphatic)oxy; aryloxy; heteroaryloxy; (araliphatic)oxy; (heteroaraliphatic)oxy; aroyl; heteroaroyl; amino; oxo (on a non-aromatic carbocyclic ring of a benzofused bicyclic or tricyclic aryl); nitro; carboxy; amido; acyl (e.g., aliphaticcarbonyl, (cycloaliphatic)carbonyl, ((cycloaliphatic)aliphatic)carbonyl, (araliphatic)carbonyl, (heterocycloaliphatic)carbonyl, ((heterocycloaliphatic)aliphatic)carbonyl, or (heteroaraliphatic)carbonyl); sulfonyl (e.g., aliphatic-S(O)₂— or amino-S(O)₂—); sulfinyl (e.g., aliphatic-S(O)— or cycloaliphatic-S(O)—); sulfonyl (e.g., aliphatic-S—); cyano; halo; hydroxy; mercapto; sulfoxy; urea; thiourea; sulfonamide; sulfamide; or carbamoyl. Alternatively, an aryl can be unsubstituted.

Non-limiting examples of substituted aryls include haloaryl (e.g., mono-, di- (e.g., p,m-dihaloaryl), and (trihalo)aryl); (carboxy)aryl (e.g., (alkoxycarbonyl)aryl, ((aralkyl)carbonyloxy)aryl, and (alkoxycarbonyl)aryl); (amido)aryl (e.g., (aminocarbonyl)aryl, (((alkylamino)alkyl)aminocarbonyl)aryl, (alkylcarbonyl)aminoaryl, (arylaminocarbonyl)aryl, and (((heteroaryl)amino)carbonyl)aryl); aminoaryl (e.g., ((alkylsulfonyl)amino)aryl or ((dialkyl)amino)aryl); (cyanoalkyl)aryl; (alkoxy)aryl; (sulfonamide)aryl (e.g., (aminosulfonyl)aryl); (alkylsulfonyl)aryl; (cyano)aryl; (hydroxyalkyl)aryl; ((alkoxy)alkyl)aryl; (hydroxy)aryl, ((carboxy)alkyl)aryl; (((dialkyl)amino)alkyl)aryl; (nitroalkyl)aryl; (((alkylsulfonyl)amino)alkyl)aryl; ((heterocycloaliphatic)carbonyl)aryl; ((alkylsulfonyl)alkyl)aryl; (cyanoalkyl)aryl; (hydroxyalkyl)aryl; (alkylcarbonyl)aryl; alkylaryl; (trihaloalkyl)aryl; p-amino-m-alkoxycarbonylaryl; p-amino-m-cyanoaryl; p-halo-m-aminoaryl; and (m-(heterocycloaliphatic)-o-(alkyl))aryl.

As used herein, an “araliphatic” such as an “aralkyl” group refers to an aliphatic group (e.g., a C₁₋₄ alkyl group) that is substituted with an aryl group. “Aliphatic,” “alkyl,” and “aryl” are as defined herein. An example of an araliphatic such as an aralkyl group is benzyl.

As used herein, an “aralkyl” group refers to an alkyl group (e.g., a C₁ alkyl group) that is substituted with an aryl group. Both “alkyl” and “aryl” have been defined above. An example of an aralkyl group is benzyl. An aralkyl is optionally substituted with one or more substituents. Each of the one or more substituents independent can be, e.g., aliphatic (e.g., alkyl, alkenyl, or alkynyl, including carboxyalkyl, hydroxyalkyl, or haloalkyl such as trifluoromethyl); cycloaliphatic (e.g., cycloalkyl or cycloalkenyl); (cycloalkyl)alkyl; heterocycloalkyl; (heterocycloalkyl)alkyl; aryl; heteroaryl; alkoxy; cycloalkyloxy; heterocycloalkyloxy; aryloxy; heteroaryloxy; aralkyloxy; heteroaralkyloxy; aroyl; heteroaroyl; nitro; carboxy; alkoxycarbonyl; alkylcarbonyloxy; amido (e.g., alkylamido, cycloalkylamido, (cycloalkylalkyl)amido, arylamido, aralkylamido, (heterocycloalkyl)amido, (heterocycloalkylalkyl)amido, heteroarylamido, or heteroaralkylamido); cyano; halo; hydroxy; acyl; mercapto; alkylsulfanyl; sulfoxy; urea; thiourea; sulfonamide; sulfamide; oxo; or carbamoyl.

As used herein, a “bicyclic ring system” includes 8- to 12- (e.g., 9-, 10-, or 11-) membered structures that form two rings, wherein the two rings have at least one atom in common (e.g., 2 atoms in common). Bicyclic ring systems include bicycloaliphatics (e.g., bicycloalkyl or bicycloalkenyl), bicycloheteroaliphatics, bicyclic aryls, and bicyclic heteroaryls.

As used herein, a “cycloaliphatic” group encompasses a “cycloalkyl” group and a “cycloalkenyl” group, each of which being optionally substituted as set forth below.

As used herein, a “cycloalkyl” group refers to a saturated carbocyclic mono- or bicyclic (fused or bridged) ring of 3 to 10 (e.g., 5 to 10) carbon atoms. Examples of cycloalkyl groups include cyclopropyl, cyclobutyl, cyclopentyl, cyclohexyl, cycloheptyl, adamantyl, norbornyl, cubyl, octahydro-indenyl, decahydro-naphthyl, bicyclo[3.2.1]octyl, bicyclo[2.2.2]octyl, bicyclo[3.3.1]nonyl, bicyclo[3.3.2.]decyl, bicyclo[2.2.2]octyl, adamantyl, azacycloalkyl, or ((aminocarbonyl)cycloalkyl)cycloalkyl. A “cycloalkenyl” group, as used herein, refers to a non-aromatic carbocyclic ring of 3-10 (e.g., 4-8) carbon atoms having one or more double bonds. Examples of cycloalkenyl groups include cyclopentenyl, 1,4-cyclohexa-di-enyl, cycloheptenyl, cyclooctenyl, hexahydro-indenyl, octahydro-naphthyl, cyclohexenyl, cyclopentenyl, bicyclo[2.2.2]octenyl, or bicyclo[3.3.1]nonenyl. A cycloalkyl or cycloalkenyl group can be optionally substituted with one or more substituents such as aliphatic (e.g., alkyl, alkenyl, or alkynyl); cycloaliphatic; (cycloaliphatic)aliphatic; heterocycloaliphatic; (heterocycloaliphatic) aliphatic; aryl; heteroaryl; alkoxy; (cycloaliphatic)oxy; (heterocycloaliphatic)oxy; aryloxy; heteroaryloxy; (araliphatic)oxy; (heteroaraliphatic)oxy; aroyl; heteroaroyl; amino; amido (e.g., (aliphatic)carbonylamino, (cycloaliphatic)carbonylamino, ((cycloaliphatic)aliphatic)carbonylamino, (aryl)carbonylamino, (araliphatic)carbonylamino, (heterocycloaliphatic)carbonylamino, ((heterocycloaliphatic)aliphatic)carbonylamino, (heteroaryl)carbonylamino, or (heteroaraliphatic)carbonylamino); nitro; carboxy (e.g., HOOC—, alkoxycarbonyl, or alkylcarbonyloxy); acyl (e.g., (cycloaliphatic)carbonyl, ((cycloaliphatic) aliphatic)carbonyl, (araliphatic)carbonyl, (heterocycloaliphatic)carbonyl, ((heterocycloaliphatic)aliphatic)carbonyl, or (heteroaraliphatic)carbonyl); cyano; halo; hydroxy; mercapto; sulfonyl (e.g., alkyl-S(O)₂— and aryl-S(O)₂—); sulfinyl (e.g., alkyl-S(O)—); sulfanyl (e.g., alkyl-S—); sulfoxy; urea; thiourea; sulfonamide; sulfamide; oxo; or carbamoyl.

As used herein, “cyclic moiety” includes cycloaliphatic, heterocycloaliphatic, aryl, or heteroaryl, each of which has been defined previously.

As used herein, the term “heterocycloaliphatic” encompasses a heterocycloalkyl group and a heterocycloalkenyl group, each of which being optionally substituted as set forth below.

As used herein, a “heterocycloalkyl” group refers to a 3-10 membered mono- or bicylic (fused or bridged) (e.g., 5- to 10-membered mono- or bicyclic) saturated ring structure, in which one or more of the ring atoms is a heteroatom (e.g., N, O, S, or combinations thereof). Examples of a heterocycloalkyl group include piperidyl, piperazyl, tetrahydropyranyl, tetrahydrofuryl, 1,4-dioxolanyl, 1,4-dithianyl, 1,3-dioxolanyl, oxazolidyl, isoxazolidyl, morpholinyl, thiomorpholyl, octahydrobenzofuryl, octahydrochromenyl, octahydrothiochromenyl, octahydroindolyl, octahydropyrindinyl, decahydroquinolinyl, octahydrobenzo[b]thiopheneyl, 2-oxa-bicyclo[2.2.2]octyl, 1-aza-bicyclo[2.2.2]octyl, 3-aza-bicyclo[3.2.1]octyl, and 2,6-dioxa-tricyclo[3.3.1.0^{3,7}]nonyl. A monocyclic heterocycloalkyl group can be fused with a phenyl moiety such as tetrahydroisoquinoline.

A “heterocycloalkenyl” group, as used herein, refers to a mono- or bicylic (e.g., 5- to 10-membered mono- or bicyclic) non-aromatic ring structure having one or more double bonds, and wherein one or more of the ring atoms is a heteroatom (e.g., N, O, or S). Monocyclic and bicycloheteroaliphatics are numbered according to standard chemical nomenclature.

A heterocycloalkyl or heterocycloalkenyl group can be optionally substituted with one or more substituents such as aliphatic (e.g., alkyl, alkenyl, or alkynyl); cycloaliphatic; (cycloaliphatic)aliphatic; heterocycloaliphatic; (heterocycloaliphatic)aliphatic; aryl; heteroaryl; alkoxy; (cycloaliphatic)oxy; (heterocycloaliphatic)oxy; aryloxy; heteroaryloxy; (araliphatic)oxy; (heteroaraliphatic)oxy; aroyl; heteroaroyl; amino; amido (e.g., (aliphatic)carbonylamino, (cycloaliphatic)carbonylamino, ((cycloaliphatic) aliphatic)carbonylamino, (aryl)carbonylamino, (araliphatic)carbonylamino, (heterocycloaliphatic)carbonylamino, ((heterocycloaliphatic) aliphatic)carbonylamino, (hetero aryl)carbonylamino, or (heteroaraliphatic)carbonylamino); nitro; carboxy (e.g., HOOC—, alkoxycarbonyl, or alkylcarbonyloxy); acyl (e.g., (cycloaliphatic)carbonyl, ((cycloaliphatic) aliphatic)carbonyl, (araliphatic)carbonyl, (heterocycloaliphatic)carbonyl, ((heterocycloaliphatic)aliphatic)carbonyl, or (heteroaraliphatic)carbonyl); nitro; cyano; halo; hydroxy; mercapto; sulfonyl (e.g., alkylsulfonyl or arylsulfonyl); sulfinyl (e.g., alkylsulfinyl); sulfanyl (e.g., alkylsulfanyl); sulfoxy; urea; thiourea; sulfonamide; sulfamide; oxo; or carbamoyl.

A “heteroaryl” group, as used herein, refers to a monocyclic, bicyclic, or tricyclic ring system having 4 to 15 ring atoms wherein at least one of the ring atoms is a heteroatom (e.g., N, O, S, or combinations thereof and in which the monocyclic ring system is aromatic or at least one of the rings in the bicyclic or tricyclic ring systems is aromatic. A heteroaryl group includes a benzofused ring system having 2 to 3 rings. For example, a benzofused group includes benzo fused with one or two 4- to 8-membered heterocycloaliphatic moieties (e.g., indolizyl, indolyl, isoindolyl, 3H-indolyl, indolinyl, benzo[b]furyl, benzo[b]thiophenyl, quinolinyl, or isoquinolinyl). Some examples of heteroaryl are azetidinyl, pyridyl, 1H-indazolyl, furyl, pyrrolyl, thienyl, thiazolyl, oxazolyl, imidazolyl, tetrazolyl, benzofuryl, isoquinolinyl, benzthiazolyl, xanthene, thioxanthene, phenothiazine, dihydroindole, benzo[1,3]dioxole, benzo[b]furyl, benzo[b]thiophenyl, indazolyl, benzimidazolyl, benzthiazolyl, puryl, cinnolyl, quinolyl, quinazolyl, cinnolyl, phthalazyl, quinazolyl, quinoxalyl, isoquinolyl, 4H-quinolizyl, benzo-1,2,5-thiadiazolyl, and 1,8-naphthyridyl.

Without limitation, examples of monocyclic heteroaryls include furyl, thiophenyl, 2H-pyrrolyl, pyrrolyl, oxazolyl, thazolyl, imidazolyl, pyrazolyl, isoxazolyl, isothiazolyl, 1,3,4-thiadiazolyl, 2H-pyranyl, 4-H-pranyl, pyridyl, pyridazyl, pyrimidyl, pyrazolyl, pyrazyl, and 1,3,5-triazyl. Monocyclic heteroaryls are numbered according to standard chemical nomenclature.

Without limitation, examples of bicyclic heteroaryls include indolizyl, indolyl, isoindolyl, 3H-indolyl, indolinyl, benzo[b]furyl, benzo[b]thiophenyl, quinolinyl, isoquinolinyl, indolizyl, isoindolyl, indolyl, benzo[b]furyl, bexo[b]thiophenyl, indazolyl, benzimidazyl, benzthiazolyl, purinyl, 4H-quinolizyl, quinolyl, isoquinolyl, cinnolyl, phthalazyl, quinazolyl, quinoxalyl, 1,8-naphthyridyl, and pteridyl. Bicyclic heteroaryls are numbered according to standard chemical nomenclature.

A heteroaryl is optionally substituted with one or more substituents such as aliphatic (e.g., alkyl, alkenyl, or alkynyl); cycloaliphatic; (cycloaliphatic)aliphatic; heterocycloaliphatic; (heterocycloaliphatic)aliphatic; aryl; heteroaryl; alkoxy; (cycloaliphatic)oxy; (heterocycloaliphatic)oxy; aryloxy; heteroaryloxy; (araliphatic)oxy; (heteroaraliphatic)oxy; aroyl; heteroaroyl; amino; oxo (on a non-aromatic carbocyclic or heterocyclic ring of a bicyclic or tricyclic heteroaryl); carboxy; amido; acyl (e.g., aliphaticcarbonyl; (cycloaliphatic)carbonyl; ((cycloaliphatic)aliphatic)carbonyl; (araliphatic)carbonyl; (heterocycloaliphatic)carbonyl; ((heterocycloaliphatic)aliphatic)carbonyl; or (heteroaraliphatic)carbonyl); sulfonyl (e.g., aliphatic-S(O)₂— or amino-S(O)₂—); sulfinyl (e.g., aliphatic-S(O)—); sulfanyl (e.g., aliphatic-S—); nitro; cyano; halo; hydroxy; mercapto; sulfoxy; urea; thiourea; sulfonamide; sulfamide; or carbamoyl. Alternatively, a heteroaryl can be unsubstituted.

Non-limiting examples of substituted heteroaryl include (halo)heteroaryl (e.g., mono- and di-(halo)heteroaryl); (carboxy)heteroaryl (e.g., (alkoxycarbonyl)heteroaryl); cyanoheteroaryl; aminoheteroaryl (e.g., ((alkylsulfonyl)amino)heteroaryl and ((dialkyl)amino)heteroaryl); (amido)heteroaryl (e.g., aminocarbonyl)heteroaryl, ((alkylcarbonyl)amino)heteroaryl, ((((alkyl)amino)alkyl)aminocarbonyl)heteroaryl, (((heteroaryl)amino)carbonyl)heteroaryl, ((heterocycloaliphatic)carbonyl)heteroaryl, and ((alkylcarbonyl)amino)heteroaryl); (cyanoalkyl)heteroaryl; (alkoxy)heteroaryl; (sulfonamide)heteroaryl (e.g., (aminosulfonyl)heteroaryl); (sulfonyl)heteroaryl (e.g., (alkyl sulfonyl)heteroaryl); (hydroxyalkyl)heteroaryl; (alkoxyalkyl)heteroaryl; (hydroxy)heteroaryl; ((carboxy)alkyl)heteroaryl; (((dialkyl)amino)alkyl)heteroaryl; (heterocycloaliphatic)heteroaryl; (cycloaliphatic)heteroaryl; (nitroalkyl)heteroaryl; (((alkylsulfonyl)amino)alkyl)heteroaryl; ((alkylsulfonyl)alkyl)heteroaryl; (cyanoalkyl)heteroaryl; (acyl)heteroaryl (e.g., (alkylcarbonyl)heteroaryl); (alkyl)heteroaryl, and (haloalkyl)heteroaryl (e.g., trihaloalkylheteroaryl).

A “heteroaraliphatic” group (e.g., a heteroaralkyl group) as used herein, refers to an aliphatic group (e.g., a C₁₋₄ alkyl or C₂₋₆ alkenyl group) that is substituted with a heteroaryl group. “Aliphatic,” “alkyl,” and “heteroaryl” have been defined above.

A “heteroaralkyl” group, as used herein, refers to an alkyl group (e.g., a C_{IA} alkyl or C₂₋₆ alkenyl group) that is substituted with a heteroaryl group. Both “alkyl” and “heteroaryl” have been defined above. A heteroaralkyl is optionally substituted with one or more substituents such as alkyl (e.g., carboxyalkyl, hydroxyalkyl, and haloalkyl such as trifluoromethyl); alkenyl; alkynyl; cycloalkyl; (cycloalkyl)alkyl; heterocycloalkyl; (heterocycloalkyl)alkyl; aryl; heteroaryl; alkoxy; cycloalkyloxy; heterocycloalkyloxy; aryloxy; heteroaryloxy; aralkyloxy; heteroaralkyloxy; aroyl; heteroaroyl; nitro; carboxy; alkoxycarbonyl; alkylcarbonyloxy; aminocarbonyl; alkylcarbonylamino; cycloalkylcarbonylamino; (cycloalkylalkyl)carbonylamino; arylcarbonylamino; aralkylcarbonylamino; (heterocycloalkyl)carbonylamino; (heterocycloalkylalkyl)carbonylamino; heteroarylcarbonylamino; heteroaralkylcarbonylamino; cyano; halo; hydroxy; acyl; mercapto; alkylsulfanyl; sulfoxy; urea; thiourea; sulfonamide; sulfamide; oxo; or carbamoyl.

As used herein, an “acyl” group refers to a formyl group or R^{X}—C(O)— (such as -alkyl-C(O)—, also referred to as “alkylcarbonyl”) where R^{X} and “alkyl” have been defined previously. Acetyl and pivaloyl are examples of acyl groups.

As used herein, an “aroyl” or “heteroaroyl” group refers to aryl-C(O)— or heteroaryl-C(O)—. The aryl and heteroaryl portion of the aroyl or heteroaroyl is optionally substituted as previously defined.

As used herein, an “alkoxy” group refers to an alkyl-O— group wherein “alkyl” has been defined previously.

As used herein, a “carbamoyl” group refers to a group having the structure —O—C(O)—N(R^{X})(R^{Y}) or —N(R^{X})—C(O)—O—R^{Z}, wherein R^{X} and R^{Y} are as defined above and R^{Z} can be aliphatic, aryl, araliphatic, heterocycloaliphatic, hetero aryl, or heteroaraliphatic.

As used herein, a “carboxy” group refers to —C(O)OH, —C(O)OR^{X}, —O—C(O)H, —O—C(O)R^{X} when used as a terminal group; or —O—C(O)— or —C(O)—O— when used as an internal group.

As used herein, a “haloaliphatic” group refers to an aliphatic group substituted with 1 to 3 halogen atoms. For instance, the term haloalkyl includes the group —CF₃.

As used herein, a “mercapto” group refers to —SH.

As used herein, a “sulfonic” group refers to —S(O)₂—OH or —S(O)₂—OR^{X} when used terminally.

As used herein, a “sulfamide” group refers to the structure —N(R^{X})—S(O)₂—N(R^{Y})(R^{Z}) when used terminally and —N(R^{X})—S(O)₂—N(R^{Y})— when used internally, wherein R^{X}, R^{Y}, and R^{Z} have been defined above.

As used herein, a “sulfonamide” group refers to the structure —S(O)₂—N(R^{X})(R^{Y}) or —N(R^{X})—S(O)₂—R^{Z} when used terminally; or —S(O)₂—N(R^{X})— or —N(R^{X})—S(O)₂— when used internally, wherein R^{X}, R^{Y}, and R^{Z} are defined above.

As used herein, a “sulfanyl” group refers to —S—R^{X} when used terminally and —S— when used internally, wherein R^{X} has been defined above. Examples of sulfanyls include aliphatic-S—, cycloaliphatic-S—, aryl-S—, or the like.

As used herein, a “sulfinyl” group refers to —S(O)—R^{X} when used terminally and —S(O)— when used internally, wherein R^{X} has been defined above. Exemplary sulfinyl groups include aliphatic-S(O)—, aryl-S(O)—, (cycloaliphatic(aliphatic))-S(O)—, cycloalkyl-S(O)—, heterocycloaliphatic-S(O)—, heteroaryl-S(O)—, or the like.

As used herein, a “sulfonyl” group refers to —S(O)₂—R^{X} when used terminally and —S(O)₂— when used internally, wherein R^{X} has been defined above. Exemplary sulfonyl groups include aliphatic-S(O)₂—, aryl-S(O)₂—, (cycloaliphatic(aliphatic))-S(O)₂—, cycloaliphatic-S(O)₂—, heterocycloaliphatic-S(O)₂—, heteroaryl-S(O)₂—, (cycloaliphatic(amido(aliphatic)))-S(O)₂—, or the like.

As used herein, a “sulfoxy” group refers to —O—SO—R^{X} or —SO—O—R^{X}, when used terminally and —O—S(O)— or —S(O)—O— when used internally, where R^{X} has been defined above.

As used herein, a “halogen” or “halo” group refers to fluorine, chlorine, bromine or iodine.

As used herein, an “alkoxycarbonyl,” which is encompassed by the term carboxy, used alone or in connection with another group refers to a group such as alkyl-O—C(O)—.

As used herein, an “alkoxyalkyl” refers to an alkyl group such as alkyl-O-alkyl-, wherein alkyl has been defined above.

As used herein, a “carbonyl” refers to —C(O)—.

As used herein, an “oxo” refers to ═O.

As used herein, an “aminoalkyl” refers to the structure (R^{X})₂N-alkyl-.

As used herein, a “cyanoalkyl” refers to the structure (NC)-alkyl-.

As used herein, a “urea” group refers to the structure —N(R^{X})—CO—N(R^{Y})(R^{Z}) and a “thiourea” group refers to the structure —N(R^{X})—CS—N(R^{Y})(R^{Z}) when used terminally and —N(R^{X})—CO—N(R^{Y})— or —N(R^{X})—CS—N(R^{Y})— when used internally, wherein R^{X}, R^{Y}, and R^{Z} have been defined above.

As used herein, a “guanidine” group refers to —N═C(N(R^{X})(R^{Y}))(N(R^{X})(R^{Y})) or —N(R^{X})═C(N(R^{X})(R^{Y}))(N(R^{X})(R^{Y})), wherein R^{X} and R^{Y} have been defined above.

As used herein, the term “amidino” refers to the structure —C(═NR^{X})(N(R^{X})(R^{Y})) wherein R^{X} and R^{Y} have been defined above.

In general, the term “vicinal” refers to the placement of substituents on a group that includes two or more carbon atoms, wherein the substituents are attached to adjacent carbon atoms.

In general, the term “geminal” refers to the placement of substituents on a group that includes two or more carbon atoms, wherein the substituents are attached to the same carbon atom.

The terms “terminally” and “internally” refer to the location of a group within a substituent. A group is terminal when the group is present at the end of the substituent not further bonded to the rest of the chemical structure. Carboxyalkyl, i.e., R^{X}O(O)C-alkyl-, is an example of a carboxy group being used terminally. A group is internal when the group is present in the middle of a substituent to at the end of the substituent bound to the rest of the chemical structure. Alkylcarboxy (e.g., alkyl-C(O)O— or alkyl-OC(O)—) and alkylcarboxyaryl (e.g., alkyl-C(O)O-aryl- or alkyl-O(CO)-aryl-) are examples of carboxy groups used internally.

As used herein, the term “cyclic group” encompasses mono-, bi-, and tri-cyclic ring systems including cycloaliphatic, heterocycloaliphatic, aryl, or heteroaryl, each of which has been previously defined.

As used herein, the term “bridged bicyclic ring system” refers to a bicyclic heterocyclicalipahtic ring system or bicyclic cycloaliphatic ring system in which the rings have at least two common atoms. Examples of bridged bicyclic ring systems include, but are not limited to, adamantanyl, norbornanyl, bicyclo[3.2.1]octyl, bicyclo[2.2.2]octyl, bicyclo[3.3.1]nonyl, bicyclo[3.2.3]nonyl, 2-oxabicyclo[2.2.2]octyl, 1-azabicyclo[2.2.2]octyl, 3-azabicyclo[3.2.1]octyl, and 2,6-dioxatricyclo[3.3.1.0^{3,7}]nonyl. A bridged bicyclic ring system can be optionally substituted with one or more substituents such as alkyl (including carboxyalkyl, hydroxyalkyl, and haloalkyl such as trifluoromethyl), alkenyl, alkynyl, cycloalkyl, (cycloalkyl)alkyl, heterocycloalkyl, (heterocycloalkyl)alkyl, aryl, heteroaryl, alkoxy, cycloalkyloxy, heterocycloalkyloxy, aryloxy, heteroaryloxy, aralkyloxy, heteroaralkyloxy, aroyl, heteroaroyl, nitro, carboxy, alkoxycarbonyl, alkylcarbonyloxy, aminocarbonyl, alkylcarbonylamino, cycloalkylcarbonylamino, (cycloalkylalkyl)carbonylamino, arylcarbonylamino, aralkylcarbonylamino, (heterocycloalkyl)carbonylamino, (heterocycloalkylalkyl)carbonylamino, heteroarylcarbonylamino, heteroaralkylcarbonylamino, cyano, halo, hydroxy, acyl, mercapto, alkylsulfanyl, sulfoxy, urea, thiourea, sulfonamide, sulfamide, oxo, or carbamoyl.

The phrase “optionally substituted” is used interchangeably with the phrase “substituted or unsubstituted.” As described herein, compounds of the invention can optionally be substituted with one or more substituents, such as are illustrated generally above, or as exemplified by particular classes, subclasses, and species of the invention. As described herein, the variables R₁, R₂, R₃, and R₄, and other variables contained therein Formula (I) encompass specific groups, such as alkyl and aryl. Unless otherwise noted, each of the specific groups for the variables R₁, R₂, R₃, and R₄, and other variables contained therein can be optionally substituted with one or more substituents described herein. Each substituent of a specific group is further optionally substituted with one to three of halo, cyano, oxoalkoxy, hydroxy, amino, nitro, aryl, haloalkyl, and alkyl. For instance, an alkyl group can be substituted with alkylsulfanyl and the alkylsulfanyl can be optionally substituted with one to three of halo, cyano, oxoalkoxy, hydroxy, amino, nitro, aryl, haloalkyl, and alkyl. As an additional example, the cycloalkyl portion of a (cycloalkyl)carbonylamino can be optionally substituted with one to three of halo, cyano, alkoxy, hydroxy, nitro, haloalkyl, and alkyl. When two alkoxy groups are bound to the same atom or adjacent atoms, the two alkxoy groups can form a ring together with the atom(s) to which they are bound.

In general, the term “substituted,” whether preceded by the term “optionally” or not, refers to the replacement of hydrogen radicals in a given structure with the radical of a specified substituent. Specific substituents are described above in the definitions and below in the description of compounds and examples thereof. Unless otherwise indicated, an optionally substituted group can have a substituent at each substitutable position of the group, and when more than one position in any given structure can be substituted with more than one substituent selected from a specified group, the substituent can be either the same or different at every position. A ring substituent, such as a heterocycloalkyl, can be bound to another ring, such as a cycloalkyl, to form a spiro-bicyclic ring system, e.g., both rings share one common atom. As one of ordinary skill in the art will recognize, combinations of substituents envisioned by this invention are those combinations that result in the formation of stable or chemically feasible compounds.

The phrase “stable or chemically feasible,” as used herein, refers to compounds that are not substantially altered when subjected to conditions to allow for their production, detection, and preferably their recovery, purification, and use for one or more of the purposes disclosed herein. In some embodiments, a stable compound or chemically feasible compound is one that is not substantially altered when kept at a temperature of 40° C. or less, in the absence of moisture or other chemically reactive conditions, for at least a week.

As used herein, a “subject” for treatment generally refers and thus may be interchangeable with a “patient,” such as an animal (e.g., a mammal such as a human).

As used herein, an “effective amount” is defined as the amount required to confer a therapeutic effect on the treated patient, and is typically determined based on age, surface area, weight, and condition of the patient. The interrelationship of dosages for animals and humans (based on milligrams per meter squared of body surface) is described by Freireich et al., Cancer Chemother. Rep., 50: 219 (1966). Body surface area may be approximately determined from height and weight of the patient. See, e.g., Scientific Tables, Geigy Pharmaceuticals, Ardsley, N.Y., 537 (1970).

Unless otherwise stated, the structures depicted herein are also meant to include all isomeric (e.g., enantiomeric, diastereomeric, and geometric (or conformational)) forms of the structure; for example, the R and S configurations for each asymmetric center, (Z) and (E) double bond isomers, and (Z) and (E) conformational isomers. Therefore, single stereochemical isomers as well as enantiomeric, diastereomeric, and geometric (or conformational) mixtures of the present compounds are within the scope of the invention. Unless otherwise stated, all tautomeric forms of the compounds of the invention are within the scope of the invention. Additionally, unless otherwise stated, structures depicted herein are also meant to include compounds that differ only in the presence of one or more isotopically enriched atoms. For example, compounds having the present structures except for the replacement of hydrogen by deuterium or tritium, or the replacement of a carbon by a ¹³C- or ¹⁴C-enriched carbon are within the scope of this invention. Such compounds are useful, for example, as analytical tools or probes in biological assays.

### DETAILED DESCRIPTION OF THE INVENTION

In general, the invention features compounds of Formula (I), which modulate the function of IRAK proteins and methods of using these compounds, e.g., for treating a condition or disease mediated by IRAK.

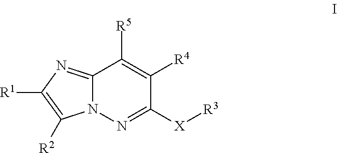

### Synthesis of Compounds of Formula (I)

Compounds of Formula (I) may be synthesized from commercially available or known starting materials by known methods. Exemplary synthetic routes to produce compounds of Formula (I) are provided in Schemes 1 and 2 below. The generic schemes are not limiting and can be applied to prepare other compounds having different variables.

In one embodiment, wherein R¹ does not contain a nitrogen atom bonded to the imidazopyridazine ring, compounds may be prepared as illustrated below in Scheme I.

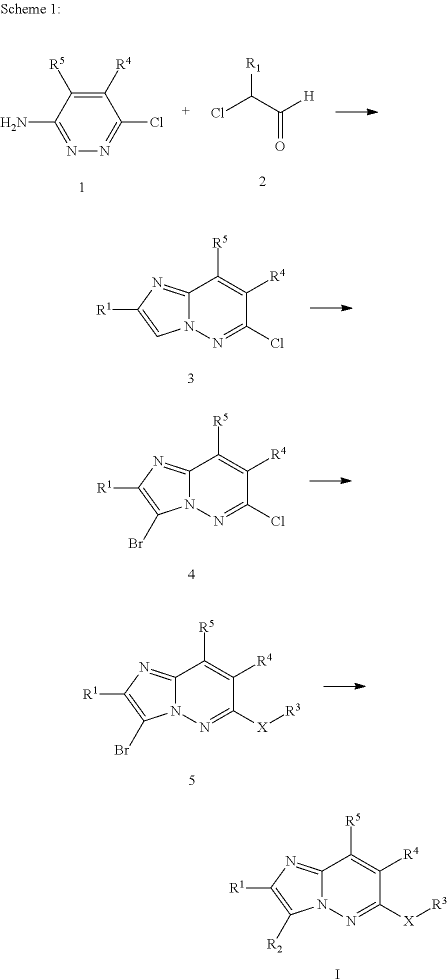

Referring to Scheme 1, an amino-chloro pyridazine of formula 1 is reacted with an α-chloroaldehyde of formula 2 in a suitable solvent such as, for example, n-butanol, to provide an imidazo[1,2-b]pyridazine of formula 3. Bromination of compound 3 with, for example, N-bromosuccinimide provides the bromo compound 4. Reaction of compound 4 with R³X—H provides the imidazopyridazine of formula 5. When X is N(R), the reaction may be conducted neat or in the presence of a suitable solvent such as t-butanol. When X is O or S, the anion R³X⁻ may be formed with a suitable base such as sodium hydride, followed by reaction of said anion with compound 4 in a suitable solvent such as, for example, dimethylformamide. The bromo compound 5 on reaction with a boronic acid R²B(OH)₂ in the presence of a palladium catalyst and an alkali metal carbonate such as sodium carbonate provides compounds of Formula (I).

An alternative method for preparing compounds of Formula (I), wherein R¹ contains a nitrogen atom bonded to the imidazopyridazine ring, is illustrated below in Scheme 2.

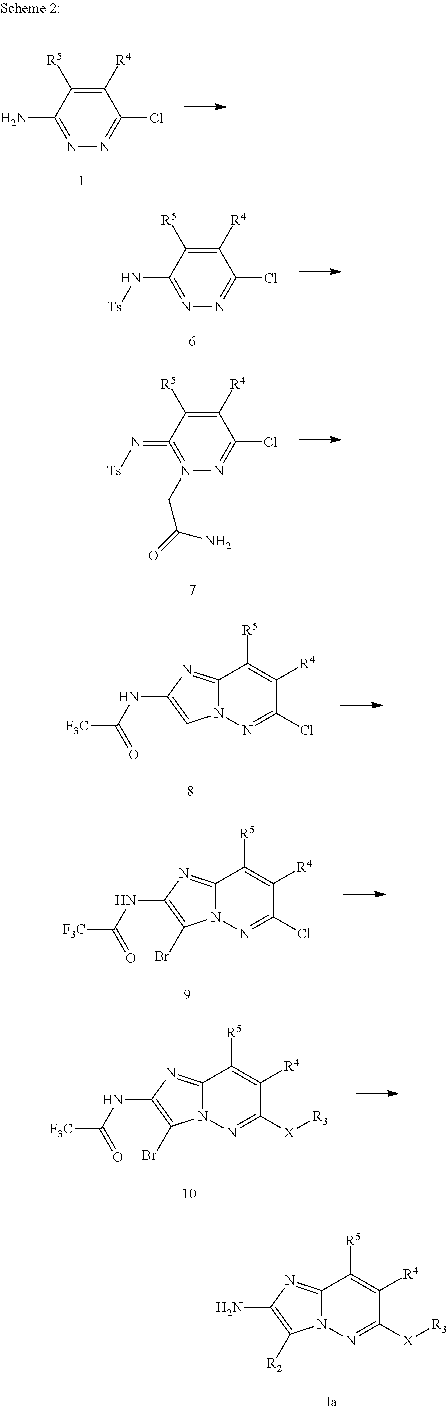

Referring to Scheme 2, reaction of the amino-chloro pyridazine of formula 1 with p-toluenesulfonyl chloride in the presence of a tertiary organic base such as, for example, pyridine provides the sulfonamide of formula 6. Reaction of compound 6 with iodoacetamide in the presence of a tertiary organic base such as, for example, di-isopropylethylamine provides the alkylated pyridazine of formula 7. Cyclization of compound 7 is achieved by reaction with trifluoroacetic acid which provides the trifluoroacetamido-imidazopyridazine of formula 8. Bromination of compound 8 with N-bromosuccinimide provides the bromo compound 9. Reaction of compound 9 with R³X—H provides the imidazopyridazine of formula 10. When X is N(R), the reaction may be conducted neat or in the presence of a suitable solvent such as t-butanol. When X is O or S, the anion R³X⁻ may be formed with a suitable base such as sodium hydride, followed by reaction of said anion with compound 9 in a suitable solvent such as, e.g., dimethylformamide. The bromo compound 9, on reaction with a boronic acid R²B(OH)₂ in the presence of a palladium catalyst and an alkali metal carbonate such as sodium carbonate, provides compounds of Formula (Ia) wherein R¹ is —NH₂. Further modification of the amino group of compound Ia using known methods such as, for example, alkylation, reductive amination, acylation or sulfonation provides additional examples of compounds of Formula (I) wherein R¹ is —N(R^{X})(R^{Y}). For a similar procedure, see, e.g., C. Hamdouchi, J. Med. Chem., 2003, 46, 4333.

### Administration of Compositions Containing Compounds of Formula (I)

As defined above, an effective amount is the amount required to confer a therapeutic effect on the treated patient. For a compound of Formula (I), an effective amount can range, for example, from about 1 mg/kg to about 150 mg/kg (e.g., from about 1 mg/kg to about 100 mg/kg). The effective amount may also vary, as recognized by those skilled in the art, dependant on route of administration, excipient usage, and the possibility of co-usage with other therapeutic treatments including use of other therapeutic agents and/or radiation therapy.

The amount of the compounds of the present invention that may be combined with the carrier materials to produce a composition in a single dosage form will vary depending upon the host treated, the particular mode of administration. For instance, the compositions may be formulated so that a dosage of between 0.01-100 mg/kg body weight/day of the modulator can be administered to a patient receiving these compositions.

It should also be understood that a specific dosage and treatment regimen for any particular patient will depend upon a variety of factors, including the activity of the specific compound employed, the age, body weight, general health, sex, diet, time of administration, rate of excretion, drug combination, and the judgment of the treating physician and the severity of the particular disease being treated. The amount of a compound of the present invention in the composition will also depend upon the particular compound in the composition.

Depending upon the particular condition, or disease, to be treated or prevented, additional therapeutic agents, which are normally administered to treat or prevent that condition, may also be present in the compositions of this invention. As used herein, additional therapeutic agents that are normally administered to treat or prevent a particular disease, or condition, are known as “appropriate for the disease, or condition, being treated.”

Compounds of Formula (I) can be administered in any manner suitable for the administration of pharmaceutical compounds, including, but not limited to, pills, tablets, capsules, aerosols, suppositories, liquid formulations for ingestion or injection or for use as eye or ear drops, dietary supplements, and topical preparations. The pharmaceutically acceptable compositions include aqueous solutions of the active agent, in an isotonic saline, 5% glucose or other well-known pharmaceutically acceptable excipient. Solubilizing agents such as cyclodextrins, or other solubilizing agents well-known to those familiar with the art, can be utilized as pharmaceutical excipients for delivery of the therapeutic compounds. As to route of administration, the compositions can be administered orally, intranasally, transdermally, intradermally, vaginally, intraaurally, intraocularly, buccally, rectally, transmucosally, or via inhalation, implantation (e.g., surgically), or intravenous administration. The compositions can be administered to an animal (e.g., a mammal such as a human, non-human primate, horse, dog, cow, pig, sheep, goat, cat, mouse, rat, guinea pig, rabbit, hamster, gerbil, or ferret, or a bird, or a reptile such as a lizard).

In certain embodiments, the compounds of Formula (I) can be administered by any method that permits the delivery of the compound to combat vascular injuries. For instance, the compounds of Formula (I) can be delivered by any method described above. Additionally, the compounds of Formula (I) can be administered by implantation (e.g., surgically) via an implantable device. Examples of implantable devices include, but are not limited to, stents, delivery pumps, vascular filters, and implantable control release compositions. Any implantable device can be used to deliver the compound provided that (i) the device, compound and any pharmaceutical composition including the compound are biocompatible, and (ii) that the device can deliver or release an effective amount of the compound to confer a therapeutic effect on the treated patient.

Delivery of therapeutic agents via stents, delivery pumps (e.g., mini-osmotic pumps), and other implantable devices is known in the art. See, e.g, Hofma, et al., Current Interventional Cardiology Reports, 3: 28-36 (2001), the entire contents of which, including references cited therein, are incorporated herein. Other descriptions of implantable devices, such as stents, can be found in U.S. Pat. Nos. 6,569,195 and 6,322,847; and PCT International Publication Numbers WO04/0044405, WO04/0018228, WO03/0229390, WO03/0228346, WO03/0225450, WO03/0216699, and WO03/0204168, each of which is also incorporated herein in by reference its entirety.

A delivery device, such as stent, includes a compound of Formula (I). The compound may be incorporated into or onto the stent using methodologies known in the art. In some embodiments, a stent can include interlocked meshed cables. Each cable can include metal wires for structural support and polyermic wires for delivering the therapeutic agent. The polymeric wire can be dosed by immersing the polymer in a solution of the therapeutic agent. Alternatively, the therapeutic agent can be embedded in the polymeric wire during the formation of the wire from polymeric precursor solutions. In other embodiments, stents or implantable devices can be coated with polymeric coatings that include the therapeutic agent. The polymeric coating can be designed to control the release rate of the therapeutic agent.

Controlled release of therapeutic agents can utilize various technologies. Devices are known having a monolithic layer or coating incorporating a heterogeneous solution and/or dispersion of an active agent in a polymeric substance, where the diffusion of the agent is rate limiting, as the agent diffuses through the polymer to the polymer-fluid interface and is released into the surrounding fluid. In some devices, a soluble substance is also dissolved or dispersed in the polymeric material, such that additional pores or channels are left after the material dissolves. A matrix device is generally diffusion limited as well, but with the channels or other internal geometry of the device also playing a role in releasing the agent to the fluid. The channels can be pre-existing channels or channels left behind by released agent or other soluble substances.

Erodible or degradable devices typically have the active agent physically immobilized in the polymer. The active agent can be dissolved and/or dispersed throughout the polymeric material. The polymeric material is often hydrolytically degraded over time through hydrolysis of labile bonds, allowing the polymer to erode into the fluid, releasing the active agent into the fluid. Hydrophilic polymers have a generally faster rate of erosion relative to hydrophobic polymers. Hydrophobic polymers are believed to have almost purely surface diffusion of active agent, having erosion from the surface inwards. Hydrophilic polymers are believed to allow water to penetrate the surface of the polymer, allowing hydrolysis of labile bonds beneath the surface, which can lead to homogeneous or bulk erosion of polymer.

The implantable device coating can include a blend of polymers each having a different release rate of the therapeutic agent. For instance, the coating can include a polylactic acid/polyethylene oxide (PLA-PEO) copolymer and a polylactic acid/polycaprolactone (PLA-PCL) copolymer. The polylactic acid/polyethylene oxide (PLA-PEO) copolymer can exhibit a higher release rate of therapeutic agent relative to the polylactic acid/polycaprolactone (PLA-PCL) copolymer. The relative amounts and dosage rates of therapeutic agent delivered over time can be controlled by controlling the relative amounts of the faster releasing polymers relative to the slower releasing polymers. For higher initial release rates the proportion of faster releasing polymer can be increased relative to the slower releasing polymer. If most of the dosage is desired to be released over a long time period, most of the polymer can be the slower releasing polymer. The stent can be coated by spraying the stent with a solution or dispersion of polymer, active agent, and solvent. The solvent can be evaporated, leaving a coating of polymer and active agent. The active agent can be dissolved and/or dispersed in the polymer. In some embodiments, the co-polymers can be extruded over the stent body.

Optionally, compounds of Formula (I) can be administered in conjunction with one or more other agents that inhibit the TGFβ signaling pathway or treat the corresponding pathological disorders (e.g., fibrosis or progressive cancers) by way of a different mechanism of action. Examples of these agents include angiotensin converting enzyme inhibitors, nonsteroid and steroid anti-inflammatory agents, as well as agents that antagonize ligand binding or activation of the TGFβ receptors, e.g., anti-TGFβ, anti-TGFβ receptor antibodies, or antagonists of the TGFβ type II receptors.

### Uses of Compounds of Formula (I)

The present invention provides a method of treating or reducing the severity of a disease in a patient by using a compound of Formula (I) as described above, wherein said disease is selected from IRAK-mediated pathologies, such as rheumatoid arthritis, multiple sclerosis, sepsis, osteoarthritis, inflammatory bowel disease, osteoporosis, myasthenia gravis, stroke, Alzheimer's disease, Parkinson's disease, cardiac contractile dysfunction, type I diabetes, type II diabetes or familial cold autoinflammatory syndrome, allergic disease, cancer, psoriasis, asthma, or graft rejection.

The efficacy of this method of treatment may be correlated to the activity of a compound of Formula (I) in modulating the kinase activity of IRAK4 to phosphorylate IRAK1 peptide, which can be determined by methods known in the art. For instance, biotin labeled IRAK1, AA358-389, can be phosphorylated (in Ser and Thr positions) by IRAK4, followed by a detection step that uses TR-FRET as the tool for detecting phosphorylation. The FRET signal is generated by a mixture of two antibodies that bind to the phosphorylated Threonines in IRAK1 (e.g., Rabbit derived polyclonal anti-p-thr and Eu-anti rabbit IgG) and SA-APC that will bind to the biotin-peptide. Eu (the donor) is excited, e.g., at 340 ηm and the fluorescence energy is transferred to APC (the acceptor), e.g., at 615 ηm, which in turn is excited and emits, e.g., at 665 ηm.

All references cited within this document are incorporated herein in their entirety by reference.

### EXAMPLES

The following examples are set forth to enable the invention described herein being more readily understood. These examples are for illustrative purposes only and are not to be construed as limiting this invention in any manner.

### Example 1

### 3-(4-fluorophenyl)-6-(tetrahydro-2H-pyran-4-yloxy)imidazo[1,2-b]pyridazine

### Step 1: 6-Chloro-imidazo[1,2-b]pyridazine

To 6-chloropyridazin-3-amine (19.3 g, 0.149 mol) in 1-butanol (150 mL) was added 26.0 mL of chloroacetaldehyde (7.0 M in water, 1.2 equiv.). The reaction was refluxed overnight and then cooled with an ice bath and the solids were filtered. The solids were washed with small amounts of cold 1-butanol and then Et₂O. 23.6 g of tan solid were recovered and dissolved in water (135 mL). A NaOH solution (1.0 N, 150 mL) was slowly added and copious solids were obtained. AcOEt (150 mL) was added and the aqueous phase was extracted with AcOEt. The organic layer was washed with a saturated solution of NaHCO₃ and then dried over MgSO₄. After evaporation, 6-chloroimidazo[1,2-b]pyridazine was obtained as a pink solid (18.1 g, 79%).

MS (ESI (+)m/z): 153.38 (M+H⁺)

¹H NMR (MeOD-d4, 300 MHz), δ 8.14 (s, 1H), 8.05 (d, J=9.3 Hz, 1H), 7.80 (s, 1H), 7.32 (d, J=9.3 Hz, 1H).

### Step 2: 3-Bromo-6-chloro-imidazo[1,2-b]pyridazine

6-Chloroimidazo[1,2-b]pyridazine (8.5 g, 0.055 mol) and N-bromosuccinimide (10.0 g, 0.056 mol) were combined in chloroform (250 mL) and refluxed for 4 hours. The reaction was cooled with an ice bath and the solids filtered. The filtrate was diluted with chloroform (150 mL) and saturated Na₂CO₃ solution (100 mL) and then vigorously stirred for an hour. The organic phase was washed with more saturated Na₂CO₃ solution and dried over MgSO₄. After evaporation, 3-bromo-6-chloro-imidazo[1,2-b]pyridazine was obtained as a tan solid (12.64 g, 98%).

MS (ESI (+)m/z): 233.87 (M+H⁺)

¹H NMR (CDCl₃-d1, 300 MHz), δ 7.83 (d, J=9.3 Hz, 1H), 7.72 (s, 1H), 7.05 (d, J=9.3 Hz, 1H).

### Step 3: 3-Bromo-6-(tetrahydropyran-4-yloxy)-imidazo[1,2-b]pyridazine

To 3-bromo-6-chloroimidazo[1,2-b]pyridazine (100.0 mg, 0.43 mmol) and tetrahydro-2H-pyran-4-ol (48 mg, 0.47 mmol) in N,N-dimethylformamide (2.0 mL) was added sodium hydride (12 mg, 0.52 mmol). The reaction was stirred at room temperature for an hour. Aqueous work-up with saturated NaHCO₃ solution and ethyl acetate was followed by drying of the organic phase over MgSO₄. After evaporation, 3-bromo-6-(tetrahydropyran-4-yloxy)-imidazo[1,2-b]pyridazine was obtained as an off-white solid (120 mg, 89%).

MS (ESI (+)m/z): 297.59 (M+H⁺)

¹H NMR (CDCl₃-d1, 300 MHz), δ 7.93 (d, J=9.6 Hz, 1H), 7.59 (s, 1H), 6.79 (d, J=9.6 Hz, 1H), 5.24 (m, 1H), 3.94 (m, 2H), 3.59 (m, 2-H), 2.13 (m, 2H), 1.83 (m, 2H).

### Step 4: 3-(4-fluorophenyl)-6-(tetrahydro-2H-pyran-4-yloxy)imidazo[1,2-b]pyridazine

To 3-bromo-6-(tetrahydropyran-4-yloxy)-imidazo[1,2-b]pyridazine (36.8 mg, 0.118 mmol) and 4-fluorophenylboronic acid (21 mg, 0.15 mmol) in dioxane (2.0 ml) was added [1,1′-bis(diphenylphosphino)ferrocene]dichloropalladium(II), complexed (1:1) with dichloromethane (24 mg, 0.030 mmol) and 2.0 M Na₂CO₃ in water (0.3 mL). The reaction was microwaved at 120° C. for 2 minutes. The reaction mixture was then neutralized with 50% HCl, filtered, concentrated, and purified by preparative HPLC to provide 3-(4-fluoro-phenyl)-6-(tetrahydropyran-4-yloxy)-imidazo[1,2-b]pyridazine as a white solid (36 mg, 93%)_{.}

MS (ESI (+)m/z): 313.82 (M+H⁺)

¹H NMR (CDCl₃-d1, 300 MHz), δ 8.59 (d, J=9.0 Hz, 1H), 7.93 (s, 1H), 7.81-7.76 (m, 2H), 7.22-7.15 (m, 3H), 5.13 (m, 1H), 3.95 (m, 2H), 3.55 (m, 2H), 2.08 (m, 2H), 1.87 (m, 2H).

### Example 2

### 4-(3-(4-fluorophenyl)imidazo[1,2-b]pyridazin-6-yl)morpholine

### Step 1: 3-Bromo-6-morpholin-4-yl-imidazo[1,2-b]pyridazine

3-Bromo-6-chloroimidazo[1,2-b]pyridazine (50.0 mg, 0.215 mmol), morpholine (70.0 mg, 0.803 mmol) and t-butyl alcohol (0.5 mL) were heated at 155° C. for 3 hours. Water (2.0 mL) was then added to the reaction mixture. After 15 minutes of additional stirring, tan solids were filtered and washed with water. Evaporation under high vacuum gave 3-bromo-6-morpholin-4-yl-imidazo[1,2-b]pyridazine as tan solids (47 mg, 75%).

MS (ESI (+)m/z): 282.56 (M+H⁺)

### Step 2: 4-(3-(4-fluorophenyl)imidazo[1,2-b]pyridazin-6-yl)morpholine

Following the procedure described in Example 1, Step 4, and replacing 3-bromo-6-(tetrahydro-pyran-4-yloxy)-imidazo[1,2-b]pyridazine with 3-bromo-6-morpholin-4-yl-imidazo[1,2-b]pyridazine, 3-(4-fluoro-phenyl)-6-morpholin-4-yl-imidazo[1,2-b]pyridazine was obtained as a white solid (29 mg, 56%).

MS (ESI (+)m/z): 298.84 (M+H⁺)

¹H NMR (MeOD-d4, 300 MHz), δ 8.21 (s, 1H), 8.14-8.07 (m, 3H), 7.71 (d, J=10.2 Hz, 1H), 7.32 (m, 2H), 3.85 (m, 4H), 3.66 (m, 4H).

### Example 3

### 3-(4-fluorophenyl)-N-(tetrahydro-2H-pyran-4-yl)imidazo[1,2-b]pyridazin-6-amine

### Step 1: (3-Bromo-imidazo[1,2-b]pyridazin-6-yl)-(tetrahydropyran-4-yl)-amine

3-Bromo-6-chloroimidazo[1,2-b]pyridazine (0.5 g, 0.002 mol) and tetrahydro-2H-pyran-4-amine (2.0 g, 0.02 mol) were heated and stirred at 160° C. in a pressure vessel for 8 hours. The reaction mixture was then pre-absorbed on 11 grams of silica using methanol and chromatographed using 400 mL of 93/6/1 methylene chloride/methanol/ammonium hydroxide to give (3-bromo-imidazo[1,2-b]pyridazin-6-yl)-(tetrahydro-pyran-4-yl)-amine as a tan solid (480 mg, 80%).

MS (ESI (+)m/z): 296.99 (M+H⁺)

¹H NMR (MeOD-d4, 300 MHz), δ 7.52 (d, J=9.6 Hz, 1H), 7.36 (s, 1H), 6.66 (d, J=9.6 Hz, 1H), 3.99-3.93 (m, 3H), 3.55 (m, 2H), 2.09 (m, 2H), 1.53 (m, 2H).

### Step 2: 3-(4-fluorophenyl)-N-(tetrahydro-2H-pyran-4-yl)-imidazo[1,2-b]pyridazin-6-amine

Following the procedure described in Example 1, Step 4, and replacing 3-bromo-6-(tetrahydro-pyran-4-yloxy)-imidazo[1,2-b]pyridazine with (3-bromo-imidazo[1,2-b]pyridazin-6-yl)-(tetrahydro-pyran-4-yl)-amine, [3-(4-fluoro-phenyl)-imidazo[1,2-b]pyridazin-6-yl]-(tetrahydro-pyran-4-yl)-amine was obtained as a white solid (30 mg, 75%).

MS (ESI (+)m/z): 312.84 (M+H⁺)

¹H NMR (CDCl₃-d1, 300 MHz), δ 8.25 (m, 1H), 7.86 (m, 2H), 7.75 (s, 1H), 7.21-7.02 (m, 3H), 5.71 (m, 1H), 4.02-3.90 (m, 3H), 3.48 (m, 2H), 2.05 (m, 2H), 1.62 (m, 2H).

### Example 4

### 4-(2-amino-6-(tetrahydro-2H-pyran-4-yloxy)imidazo[1,2-b]pyridazin-3-yl)benzonitrile

### Step 1: N-(6-Chloro-pyridazin-3-yl)-4-methyl-benzenesulfonamide

Into a 500 mL round-bottom flask, dry pyridine (128 mL, 1.58 mol) was added to 6-chloropyridazin-3-amine (10.1 g, 0.078 mol) to give an orange suspension that was stirred for 5 minutes under nitrogen. p-Toluenesulfonyl chloride (16.2 g, 0.085 mol) was then added portionwise. The reaction mixture was stirred at 85° C. for 15 hours under nitrogen. The volatiles were evaporated and cold water (150 mL) and dichloromethane (150 mL) were added. The dichloromethane was evaporated and a precipitate was observed. The solids were filtered, washed with cold water and recrystallized in ethylacetate (yellow solid, 23.2 g). N-(6-chloro-pyridazin-3-yl)-4-methyl-benzenesulfonamide was used in next step without further purification (purity=80% by LCMS at 254 nm).

MS (ESI (+)m/z): 283.52 (M+H⁺)

### Step 2: 2-(3-chloro-6-(tosylimino)pyridazin-1(6H)-yl)acetamide

The crude solid (0.5 g, purity=80% by LCMS at 254 nm) from Example 4, Step 1, was dissolved in DMF (5.0 mL) under an atmosphere of nitrogen. N,N-diisopropylethylamine (0.4 mL, 2.0 mol) was added and the reaction mixture was stirred for 5 minutes. Iodoacetamide (358 mg, 1.9 mmol) was then added at once and the reaction mixture turned from orange to red. After stirring for 3 hours at room temperature, the reaction mixture was poured onto 50 mL of water and stirred for 1 hour. The precipitate was filtered, washed with minimum water and dried with air and under vacuum to give 2-[3-Chloro-6-(toluene-4-sulfonylmethylene)-6H-pyridazin-1-yl]-acetamide as a brownish solid (600 mg, purity=72% by LCMS at 254 nm) which was used in next step without further purification.

MS (ESI (+)m/z): 340.95 (M+H⁺)

### Step 3: N-(6-chloroimidazo[1,2-b]pyridazin-2-yl)-2,2,2-trifluoroacetamide

The crude solid (0.6 g, purity=72% by LCMS at 254 nm) from Example 4, Step 2, was suspended in dry methylene chloride (6.0 mL) under nitrogen. Trifluoroacetic anhydride (4.0 mL, 0.028 mol) was then added, and the reaction mixture was heated to reflux for 3 hours under a nitrogen atmosphere. The volatiles were evaporated and the crude was cooled down in an ice bath. Ice and ethyl acetate (15 mL) were then slowly added to quench the reaction followed by addition of saturated NaHCO₃ solution (15 mL). The organic phase was washed with saturated NaHCO₃ solution, water and brine, and then dried over MgSO₄. The crude was purified by preparative HPLC to give N-(6-chloro-imidazo[1,2-b]pyridazin-2-yl)-2,2,2-trifluoro-acetamide as purple solids (195 mg, 44% for Steps 1, 2 and 3).

MS (ESI (+)m/z): 264.56 (M+H⁺)

¹H NMR (CDCl₃-d1, 300 MHz), δ 8.47 (s, 1H), 7.92 (m, 1H), 7.21 (m, 1H).

### Step 4: N-(3-Bromo-6-chloro-imidazo[1,2-b]pyridazin-2-yl-2,2,2-trifluoroacetamide

In a microwave vial, chloroform (12.0 ml) was added to a mixture of N-(6-chloroimidazo[1,2-b]pyridazin-2-yl)-2,2,2-trifluoro-acetamide (1.8 g, 6.8 mmol) and N-bromosuccinimide (1.2 g, 6.8 mmol). The reaction mixture was heated in the microwave at 100° C. for 2 minutes for two times. Isolation of the product was achieved as described in Example 1, Step 2, to provide N-(3-bromo-6-chloroimidazo[1,2-b]pyridazin-2-yl)-2,2,2-trifluoro-acetamide as a tan solid (2.2 g, 94%).

MS (ESI (+)m/z): 344.44 (M+H⁺)

¹H NMR (MeOD-d4, 300 MHz), δ 8.05 (d, J=9.6 Hz, 1H), 7.43 (d, J=9.6 Hz, 1H).

### Step 5: N-(3-bromo-6-(tetrahydro-2H-pyran-4-yloxy)imidazo[1,2-b]pyridazin-2-yl)-2,2,2-trifluoroacetamide

Following the procedure described in Example 1, Step 3, and replacing 3-bromo-6-chloro-imidazo[1,2-b]pyridazine with N-(3-bromo-6-chloro-imidazo[1,2-b]pyridazin-2-yl)-2,2,2-trifluoro-acetamide, N-[3-bromo-6-(tetrahydropyran-4-yloxy)-imidazo[1,2-b]pyridazin-2-yl]-2,2,2-trifluoro-acetamide was obtained as a white solid (591 mg, quantitative yield).

MS (ESI (+)m/z): 408.61 (M+H⁺)

¹H NMR (MeOD-d4, 300 MHz), δ 7.75 (d, J=9.6 Hz, 1H), 6.86 (d, J=9.6 Hz, 1H), 5.18 (m, 1H), 3.87 (m, 2H), 3.54 (m, 2H), 2.08 (m, 2H), 1.76 (m, 2H).

### Step 6: 4-(2-amino-6-(tetrahydro-2H-pyran-4-yloxy)imidazo[1,2-b]pyridazin-3-yl)-benzonitrile

To N-[3-Bromo-6-(tetrahydro-pyran-4-yloxy)-imidazo[1,2-b]pyridazin-2-yl]-2,2,2-trifluoro-acetamide (30 mg, 0.07 mmol) and 4-cyanobenzeneboronic acid (12 mg, 0.08 mmol) in dioxane (1.0 mL) was added [1,1′-Bis(diphenylphosphino)ferrocene]-dichloropalladium(II), complexed (1:1) with dichloromethane (8.0 mg, 0.01 mmol) and 2.0 M Na₂CO₃ in water (0.15 mL). The reaction was heated in the microwave at 150° C. for 2 minutes. The reaction mixture was neutralize with 50% HCl, filtered, concentrated and purified by preparative HPLC to provide 4-[2-Amino-6-(tetrahydro-pyran-4-yloxy)-imidazo[1,2-b]pyridazin-3-yl]-benzonitrile (white solid, 15 mg, 60%).

MS (ESI (+)m/z): 335.87 (M+H⁺)

¹H NMR (MeOD-d4, 300 MHz), δ 7.96-7.91 (m, 3H), 7.83 (m, 2H), 7.12 (d, J=9.6 Hz, 1H), 5.07 (m, 1H), 3.87 (m, 2H), 3.52 (m, 2H), 2.03 (m, 2H), 1.75 (m, 2H).

Additional examples as prepared by the methods described in Examples 1 through 4 are listed in Table 1

TABLE 1

| | | Prepared by | | |
|---|---|---|---|---|
| Example | | the Method | M.W. | M.W. |
| No. | Compound | of Example # | (calc.) | (found) |
| 5 | 6-(tetrahydro-2H-pyran-4- | 1 | 219.244 | 219.36 |
| | yloxy)imidazo[1,2-b]pyridazine | | | |
| 6 | 6-(furan-2-ylmethoxy)imidazo[1,2- | 1 | 215.212 | 215.39 |
| | b]pyridazine | | | |
| 7 | N-cyclohexylimidazo[1,2-b]pyridazin-6- | 3 | 216.288 | 216.66 |
| | amine | | | |
| 8 | 3-bromo-N-cyclohexylimidazo[1,2- | 3 | 295.189 | 294.63 |
| | b]pyridazin-6-amine | | | |
| 9 | 3-(imidazo[1,2-b]pyridazin-6- | 3 | 271.123 | 270.54 |
| | ylamino)propan-1-ol | | | |
| 10 | 3-bromo-6-(tetrahydro-2H-pyran-4- | 1 | 298.145 | 297.59 |
| | yloxy)imidazo[1,2-b]pyridazine | | | |
| 11 | 3-bromo-6-(furan-2-ylmethoxy)imidazo[1,2- | 1 | 294.113 | 295.59 |
| | b]pyridazine | | | |
| 12 | 4-(3-bromoimidazo[1,2-b]pyridazin-6- | 3 | 311.188 | 312.61 |
| | ylamino)cyclohexanol | | | |
| 13 | ethyl 3-(6-(cyclohexylamino)imidazo[1,2- | 3 | 364.449 | 364.78 |
| | b]pyridazin-3-yl)benzoate | | | |
| 14 | (3-(6-(cyclohexylamino)imidazo[1,2- | 3 | 322.412 | 322.89 |
| | b]pyridazin-3-yl)phenyl)methanol | | | |
| 15 | 3-(6-(cyclohexylamino)imidazo[1,2- | 3 | 336.395 | 336.87 |
| | b]pyridazin-3-yl)benzoic acid | | | |
| 16 | 4-(6-(furan-2-ylmethoxy)imidazo[1,2- | 1 | 307.309 | 308 |
| | b]pyridazin-3-yl)phenol | | | |
| 17 | 4-(6-(tetrahydro-2H-pyran-4- | 1 | 311.341 | 312.03 |
| | yloxy)imidazo[1,2-b]pyridazin-3-yl)phenol | | | |
| 18 | ethyl 3-(6-(tetrahydro-2H-pyran-4- | 1 | 367.405 | 368.23 |
| | yloxy)imidazo[1,2-b]pyridazin-3- | | | |
| | yl)benzoate | | | |
| 19 | N-(3-(6-(tetrahydro-2H-pyran-4- | 3 | 351.41 | 351.99 |
| | ylamino)imidazo[1,2-b]pyridazin-3- | | | |
| | yl)phenyl)acetamide | | | |
| 20 | 3-(5-methoxypyridin-3-yl)-N-(tetrahydro- | 3 | 325.372 | 326.22 |
| | 2H-pyran-4-yl)imidazo[1,2-b]pyridazin-6- | | | |
| | amine | | | |
| 21 | (E)-3-(hex-1-enyl)-N-(tetrahydro-2H-pyran- | 3 | 300.406 | 301.03 |
| | 4-yl)imidazo[1,2-b]pyridazin-6-amine | | | |
| 22 | 4-(6-(3-hydroxypropylamino)imidazo[1,2- | 3 | 314.345 | 314.99 |
| | b]pyridazin-3-yl)-2-methoxyphenol | | | |
| 23 | 4-(6-(furan-2-ylmethylamino)imidazo[1,2- | 3 | 306.325 | 307.31 |
| | b]pyridazin-3-yl)phenol | | | |
| 24 | ethyl 3-(6-(3- | 3 | 340.383 | 340.87 |
| | hydroxypropylamino)imidazo[1,2- | | | |
| | b]pyridazin-3-yl)benzoate | | | |
| 25 | 4-(6-(3-hydroxypropylamino)imidazo[1,2- | 3 | 284.319 | 284.73 |
| | b]pyridazin-3-yl)phenol | | | |
| 26 | 4-(6-(4- | 3 | 324.384 | 324.88 |
| | hydroxycyclohexylamino)imidazo[1,2- | | | |
| | b]pyridazin-3-yl)phenol | | | |
| 27 | 4-(3-(3- | 3 | 338.411 | 338.87 |
| | (hydroxymethyl)phenyl)imidazo[1,2- | | | |
| | b]pyridazin-6-ylamino)cyclohexanol | | | |
| 28 | 3-(6-(4- | 3 | 352.394 | 352.86 |
| | hydroxycyclohexylamino)imidazo[1,2- | | | |
| | b]pyridazin-3-yl)benzoic acid | | | |
| 29 | 4-(6-(isopropylamino)imidazo[1,2- | 3 | 254.293 | 254.72 |
| | b]pyridazin-3-yl)phenol | | | |
| 30 | 4-(6-(tetrahydro-2H-pyran-4- | 3 | 310.357 | 310.83 |
| | ylamino)imidazo[1,2-b]pyridazin-3- | | | |
| | yl)phenol | | | |
| 31 | 3-(6-(3-hydroxypropylamino)imidazo[1,2- | 3 | 312.329 | 312.97 |
| | b]pyridazin-3-yl)benzoic acid | | | |
| 32 | 3-(6-(cyclohexylamino)imidazo[1,2- | 3 | 335.411 | 336.02 |
| | b]pyridazin-3-yl)benzamide | | | |
| 33 | 3-(3-(3- | 3 | 298.346 | 298.76 |
| | (hydroxymethyl)phenyl)imidazo[1,2- | | | |
| | b]pyridazin-6-ylamino)propan-1-ol | | | |
| 34 | 4-(6-(tetrahydro-2H-pyran-4- | 3 | 319.368 | 320.12 |
| | ylamino)imidazo[1,2-b]pyridazin-3- | | | |
| | yl)benzonitrile | | | |
| 35 | 3-(6-(3-hydroxypropylamino)-imidazo[1,2- | 3 | 311.345 | 311.6 |
| | b]pyridazin-3-yl)benzamide | | | |
| 36 | 3-bromo-N-(tetrahydro-2H-pyran-4- | 3 | 297.161 | 296.99 |
| | yl)imidazo[1,2-b]pyridazin-6-amine | | | |
| 37 | N-cyclohexyl-3-(4-fluorophenyl)- | 3 | 310.376 | 310.84 |
| | imidazo[1,2-b]pyridazin-6-amine | | | |
| 38 | 4-(6-(cyclohexylamino)imidazo[1,2- | 3 | 317.396 | 317.86 |
| | b]pyridazin-3-yl)benzonitrile | | | |
| 39 | N-cyclohexyl-3-(4- | 3 | 322.412 | 322.78 |
| | methoxyphenyl)imidazo[1,2-b]pyridazin-6- | | | |
| | amine | | | |
| 40 | 1-(3-(6-(tetrahydro-2H-pyran-4- | 3 | 336.395 | 336.81 |
| | ylamino)imidazo[1,2-b]pyridazin-3- | | | |
| | yl)phenyl)ethanone | | | |
| 41 | 3-(4-(methoxymethoxy)phenyl)-N- | 3 | 354.41 | 354.85 |
| | (tetrahydro-2H-pyran-4-yl)imidazo[1,2- | | | |
| | b]pyridazin-6-amine | | | |
| 42 | 3-(4-(dimethylamino)phenyl)-N-(tetrahydro- | 3 | 337.427 | 337.91 |
| | 2H-pyran-4-yl)imidazo[1,2-b]pyridazin-6- | | | |
| | amine | | | |
| 43 | 4-(6-(tetrahydro-2H-pyran-4- | 3 | 322.368 | 322.79 |
| | ylamino)imidazo[1,2-b]pyridazin-3- | | | |
| | yl)benzaldehyde | | | |
| 44 | 3-(3,4-dimethoxyphenyl)-N-(tetrahydro-2H- | 3 | 354.41 | 354.83 |
| | pyran-4-yl)imidazo[1,2-b]pyridazin-6-amine | | | |
| 45 | 3-(2-methoxypyrimidin-5-yl)-N-(tetrahydro- | 3 | 326.36 | 326.83 |
| | 2H-pyran-4-yl)imidazo[1,2-b]pyridazin-6- | | | |
| | amine | | | |
| 46 | 4-(6-(cyclohexylamino)imidazo[1,2- | 3 | 308.385 | 309.11 |
| | b]pyridazin-3-yl)phenol | | | |
| 47 | 4-(6-(1-hydroxy-3-methylbutan-2- | 3 | 342.399 | 342.89 |
| | ylamino)imidazo[1,2-b]pyridazin-3-yl)-2- | | | |
| | methoxyphenol | | | |
| 48 | 3-(4-methoxypyridin-3-yl)-N-(tetrahydro- | 3 | 325.372 | 325.87 |
| | 2H-pyran-4-yl)imidazo[1,2-b]pyridazin-6- | | | |
| | amine | | | |
| 49 | 3-(1-methyl-1H-indol-5-yl)-N-(tetrahydro- | 3 | 347.422 | 347.9 |
| | 2H-pyran-4-yl)imidazo[1,2-b]pyridazin-6- | | | |
| | amine | | | |
| 50 | tert-butyl 2-(6-(tetrahydro-2H-pyran-4- | 3 | 433.512 | 434.06 |
| | ylamino)imidazo[1,2-b]pyridazin-3-yl)-1H- | | | |
| | indole-1-carboxylate | | | |
| 51 | 3-(1H-indol-2-yl)-N-(tetrahydro-2H-pyran- | 3 | 333.395 | 334.05 |
| | 4-yl)imidazo[1,2-b]pyridazin-6-amine | | | |
| 52 | 3-(6-(tetrahydro-2H-pyran-4- | 3 | 319.368 | 319.72 |
| | ylamino)imidazo[1,2-b]pyridazin-3- | | | |
| | yl)benzonitrile | | | |
| 53 | 3-(4-(methylsulfonyl)phenyl)-N-(tetrahydro- | 3 | 372.447 | 372.92 |
| | 2H-pyran-4-yl)imidazo[1,2-b]pyridazin-6- | | | |
| | amine | | | |
| 54 | N-(tetrahydro-2H-pyran-4-yl)-3-(4- | 3 | 320.396 | 320.7 |
| | vinylphenyl)imidazo[1,2-b]pyridazin-6- | | | |
| | amine | | | |
| 55 | 3-(4-ethynylphenyl)-N-(tetrahydro-2H- | 3 | 318.38 | 318.72 |
| | pyran-4-yl)imidazo[1,2-b]pyridazin-6-amine | | | |
| 56 | 3-(2-methoxyphenyl)-N-(tetrahydro-2H- | 3 | 324.384 | 324.79 |
| | pyran-4-yl)imidazo[1,2-b]pyridazin-6-amine | | | |
| 57 | 2-(6-(tetrahydro-2H-pyran-4- | 3 | 310.357 | 310.77 |
| | ylamino)imidazo[1,2-b]pyridazin-3- | | | |
| | yl)phenol | | | |
| 58 | 2-methoxy-4-(6-(tetrahydro-2H-pyran-4- | 3 | 340.383 | 340.68 |
| | ylamino)imidazo[1,2-b]pyridazin-3- | | | |
| | yl)phenol | | | |
| 59 | 2-(3-(4-fluorophenyl)imidazo[1,2- | 3 | 314.364 | 314.51 |
| | b]pyridazin-6-ylamino)-3-methylbutan-1-ol | | | |
| 60 | 4-(6-(1-hydroxy-3-methylbutan-2- | 3 | 321.384 | 321.63 |
| | ylamino)imidazo[1,2-b]pyridazin-3- | | | |
| | yl)benzonitrile | | | |
| 61 | N-(tetrahydro-2H-pyran-4-yl)-3- | 3 | 244.298 | 244.74 |
| | vinylimidazo[1,2-b]pyridazin-6-amine | | | |
| 62 | 4-(6-(1-hydroxy-3-methylbutan-2- | 3 | 342.399 | 342.82 |
| | ylamino)imidazo[1,2-b]pyridazin-3-yl)-2- | | | |
| | methoxyphenol | | | |
| 63 | 4-(6-(1-hydroxy-3-methylbutan-2- | 3 | 324.384 | 324.81 |
| | ylamino)imidazo[1,2-b]pyridazin-3- | | | |
| | yl)benzaldehyde | | | |
| 64 | 4-(6-(1-hydroxy-3-methylbutan-2- | 3 | 312.373 | 313.04 |
| | ylamino)imidazo[1,2-b]pyridazin-3- | | | |
| | yl)phenol | | | |
| 65 | 3-(6-fluoropyridin-3-yl)-N-(tetrahydro-2H- | 3 | 313.336 | 314.19 |
| | pyran-4-yl)imidazo[1,2-b]pyridazin-6-amine | | | |
| 66 | N-(tetrahydro-2H-pyran-4-yl)-3-(4- | 3 | 362.355 | 363.22 |
| | (trifluoromethyl)phenyl)imidazo[1,2- | | | |
| | b]pyridazin-6-amine | | | |
| 67 | 2-(6-(tetrahydro-2H-pyran-4- | 3 | 319.368 | 320.2 |
| | ylamino)imidazo[1,2-b]pyridazin-3- | | | |
| | yl)benzonitrile | | | |
| 68 | 3-(4-nitrophenyl)-N-(tetrahydro-2H-pyran- | 3 | 339.355 | 340.21 |
| | 4-yl)imidazo[1,2-b]pyridazin-6-amine | | | |
| 69 | 4-oxo-4-(4-(6-(tetrahydro-2H-pyran-4- | 3 | 409.446 | 410.23 |
| | ylamino)imidazo[1,2-b]pyridazin-3- | | | |
| | yl)phenylamino)butanoic acid | | | |
| 70 | N-(4-(6-(tetrahydro-2H-pyran-4- | 3 | 351.41 | 352.24 |
| | ylamino)imidazo[1,2-b]pyridazin-3- | | | |
| | yl)phenyl)acetamide | | | |
| 71 | N-(tetrahydro-2H-pyran-4-yl)-3-(thiophen- | 3 | 300.384 | 301.18 |
| | 3-yl)imidazo[1,2-b]pyridazin-6-amine | | | |
| 72 | 3-(4-(methylthio)phenyl)-N-(tetrahydro-2H- | 3 | 340.449 | 341.25 |
| | pyran-4-yl)imidazo[1,2-b]pyridazin-6-amine | | | |
| 73 | 2-(4-(6-(tetrahydro-2H-pyran-4- | 3 | 333.395 | 334.26 |
| | ylamino)imidazo[1,2-b]pyridazin-3- | | | |
| | yl)phenyl)acetonitrile | | | |
| 74 | 3-(4-(aminomethyl)phenyl)-N-(tetrahydro- | 3 | 323.4 | 324.26 |
| | 2H-pyran-4-yl)imidazo[1,2-b]pyridazin-6- | | | |
| | amine | | | |
| 75 | N-methyl-3-(6-(tetrahydro-2H-pyran-4- | 3 | 351.41 | 352.24 |
| | ylamino)imidazo[1,2-b]pyridazin-3- | | | |
| | yl)benzamide | | | |
| 76 | 3-(quinoxalin-6-yl)-N-(tetrahydro-2H- | 3 | 346.394 | 347.27 |
| | pyran-4-yl)imidazo[1,2-b]pyridazin-6-amine | | | |
| 77 | 1-(5-(6-(tetrahydro-2H-pyran-4- | 3 | 342.421 | 343.21 |
| | ylamino)imidazo[1,2-b]pyridazin-3- | | | |
| | yl)thiophen-2-yl)ethanone | | | |
| 78 | 2-fluoro-5-(6-(tetrahydro-2H-pyran-4- | 3 | 337.358 | 338.25 |
| | ylamino)imidazo[1,2-b]pyridazin-3- | | | |
| | yl)benzonitrile | | | |
| 79 | 2-fluoro-5-(6-(tetrahydro-2H-pyran-4- | 3 | 340.358 | 341.25 |
| | ylamino)imidazo[1,2-b]pyridazin-3- | | | |
| | yl)benzaldehyde | | | |
| 80 | 3-(3,4-dichlorophenyl)-N-(tetrahydro-2H- | 3 | 363.248 | 363.15 |
| | pyran-4-yl)imidazo[1,2-b]pyridazin-6-amine | | | |
| 81 | (5-(6-(tetrahydro-2H-pyran-4- | 3 | 330.41 | 331.25 |
| | ylamino)imidazo[1,2-b]pyridazin-3- | | | |
| | yl)thiophen-2-yl)methanol | | | |
| 82 | 2-(4-(6-(tetrahydro-2H-pyran-4- | 3 | 453.502 | 454.24 |
| | ylamino)imidazo[1,2-b]pyridazin-3- | | | |
| | yl)benzyl)isoindoline-1,3-dione | | | |
| 83 | piperidin-1-yl(4-(6-(tetrahydro-2H-pyran-4- | 3 | 405.502 | 406.31 |
| | ylamino)imidazo[1,2-b]pyridazin-3- | | | |
| | yl)phenyl)methanone | | | |
| 84 | 3-(3-(piperidin-1-yl)phenyl)-N-(tetrahydro- | 3 | 377.492 | 378.33 |
| | 2H-pyran-4-yl)imidazo[1,2-b]pyridazin-6- | | | |
| | amine | | | |
| 85 | 3-(4-(morpholinomethyl)phenyl)-N- | 3 | 393.491 | 394.21 |
| | (tetrahydro-2H-pyran-4-yl)imidazo[1,2- | | | |
| | b]pyridazin-6-amine | | | |
| 86 | N-(4-(6-(tetrahydro-2H-pyran-4- | 3 | 401.489 | 402.25 |
| | ylamino)imidazo[1,2-b]pyridazin-3- | | | |
| | yl)benzyl)methanesulfonamide | | | |
| 87 | 3-(benzo[c][1,2,5]oxadiazol-5-yl)-N- | 3 | 336.355 | 337.27 |
| | (tetrahydro-2H-pyran-4-yl)imidazo[1,2- | | | |
| | b]pyridazin-6-amine | | | |
| 88 | N-cyclohexyl-3-(4-fluorophenyl)-N- | 2 | 324.403 | 324.85 |
| | methylimidazo[1,2-b]pyridazin-6-amine | | | |
| 89 | 3-(3-(4-fluorophenyl)imidazo[1,2- | 2 | 286.31 | 286.8 |
| | b]pyridazin-6-ylamino)propan-1-ol | | | |
| 90 | 2-(3-(4-fluorophenyl)imidazo[1,2- | 2 | 272.283 | 272.81 |
| | b]pyridazin-6-ylamino)ethanol | | | |
| 91 | 1-(5-(6-(1-hydroxy-3-methylbutan-2- | 2 | 344.437 | 344.86 |
| | ylamino)imidazo[1,2-b]pyridazin-3- | | | |
| | yl)thiophen-2-yl)ethanone | | | |
| 92 | N-benzyl-3-(4-fluorophenyl)imidazo[1,2- | 2 | 318.355 | 318.9 |
| | b]pyridazin-6-amine | | | |
| 93 | N-(cyclohexylmethyl)-3-(4- | 2 | 324.403 | 325.25 |
| | fluorophenyl)imidazo[1,2-b]pyridazin-6- | | | |
| | amine | | | |
| 94 | 4-(6-(cyclohexylthio)imidazo[1,2- | 1 | 334.445 | 334.83 |
| | b]pyridazin-3-yl)benzonitrile | | | |
| 95 | 6-(cyclohexyloxy)-3-(4- | 1 | 311.36 | 311.62 |
| | fluorophenyl)imidazo[1,2-b]pyridazine | | | |
| 96 | 4-(6-(cyclohexyloxy)imidazo[1,2- | 1 | 318.38 | 318.88 |
| | b]pyridazin-3-yl)benzonitrile | | | |
| 97 | 4-(6-(tetrahydro-2H-pyran-4- | 1 | 320.352 | 320.82 |
| | yloxy)imidazo[1,2-b]pyridazin-3- | | | |
| | yl)benzonitrile | | | |
| 98 | N-(3-bromo-6-(tetrahydro-2H-pyran-4- | 4 | 409.167 | 408.61 |
| | yloxy)imidazo[1,2-b]pyridazin-2-yl)-2,2,2- | | | |
| | trifluoroacetamide | | | |
| 99 | 3-(4-fluorophenyl)-6-(tetrahydro-2H-pyran- | 4 | 328.347 | 328.89 |
| | 4-yloxy)imidazo[1,2-b]pyridazin-2-amine | | | |
| 100 | 4-(2-amino-6-(tetrahydro-2H-pyran-4- | 4 | 326.356 | 328.88 |
| | yloxy)imidazo[1,2-b]pyridazin-3-yl)phenol | | | |
| 101 | (E)-methyl 3-(3-(6-(tetrahydro-2H-pyran-4- | 2 | 378.432 | 378.96 |
| | ylamino)imidazo[1,2-b]pyridazin-3- | | | |
| | yl)phenyl)acrylate | | | |
| 102 | (E)-3-(3-(6-(tetrahydro-2H-pyran-4- | 2 | 364.405 | 364.94 |
| | ylamino)imidazo[1,2-b]pyridazin-3- | | | |
| | yl)phenyl)acrylic acid | | | |
| 103 | 6-(cyclohexylthio)-3-(4- | 1 | 327.425 | 327.78 |
| | fluorophenyl)imidazo[1,2-b]pyridazine | | | |
| 104 | 3-(1,4-dioxaspiro[4.5]dec-7-en-8-yl)-N- | 2 | 356.426 | 356.95 |
| | (tetrahydro-2H-pyran-4-yl)imidazo[1,2- | | | |
| | b]pyridazin-6-amine | | | |
| 105 | tert-butyl 2-(6-(tetrahydro-2H-pyran-4- | 2 | 383.452 | 384.02 |
| | ylamino)imidazo[1,2-b]pyridazin-3-yl)-1H- | | | |
| | pyrrole-1-carboxylate | | | |
| 106 | 3-(1H-pyrrol-2-yl)-N-(tetrahydro-2H-pyran- | 2 | 283.335 | 283.71 |
| | 4-yl)imidazo[1,2-b]pyridazin-6-amine | | | |
| 107 | 3-(4-(2H-1,2,3-triazol-4-yl)phenyl)-N- | 2 | 361.409 | 361.8 |
| | (tetrahydro-2H-pyran-4-yl)imidazo[1,2- | | | |
| | b]pyridazin-6-amine | | | |
| 108 | 3-(4-(2H-tetrazol-5-yl)phenyl)-N- | 2 | 362.397 | 362.86 |
| | (tetrahydro-2H-pyran-4-yl)imidazo[1,2- | | | |
| | b]pyridazin-6-amine | | | |
| 109 | (E)-3-(3-(6-(tetrahydro-2H-pyran-4- | 2 | 363.421 | 363.97 |
| | ylamino)imidazo[1,2-b]pyridazin-3- | | | |
| | yl)phenyl)acrylamide | | | |
| 110 | 3-(4-fluorophenyl)-6- | 1 | 259.306 | 260.26 |
| | (methylthio)imidazo[1,2-b]pyridazine | | | |
| 111 | 4-(6-(tetrahydro-2H-pyran-4- | 2 | 336.399 | 336.84 |
| | ylamino)imidazo[1,2-b]pyridazin-3- | | | |
| | yl)benzimidamide | | | |
| 112 | 5-(6-(tetrahydro-2H-pyran-4- | 2 | 328.394 | 328.94 |
| | ylamino)imidazo[1,2-b]pyridazin-3- | | | |
| | yl)thiophene-2-carbaldehyde | | | |
| 113 | 3-(4-fluorophenyl)imidazo[1,2-b]pyridazin- | 2 | 228.23 | 229.17 |
| | 6-amine | | | |
| 114 | 3-(4-(pent-1-ynyl)phenyl)-N-(tetrahydro- | 2 | 360.461 | 361.26 |
| | 2H-pyran-4-yl)imidazo[1,2-b]pyridazin-6- | | | |
| | amine | | | |
| 115 | 3-(1H-indazol-6-yl)-N-(tetrahydro-2H- | 2 | 334.383 | 335.31 |
| | pyran-4-yl)imidazo[1,2-b]pyridazin-6-amine | | | |
| 116 | 3-(benzofuran-5-yl)-N-(tetrahydro-2H- | 2 | 334.379 | 335.31 |
| | pyran-4-yl)imidazo[1,2-b]pyridazin-6-amine | | | |
| 117 | N-(2-(dimethylamino)ethyl)-3-(6- | 2 | 408.506 | 409.32 |
| | (tetrahydro-2H-pyran-4- | | | |
| | ylamino)imidazo[1,2-b]pyridazin-3- | | | |
| | yl)benzamide | | | |
| 118 | (4-methylpiperazin-1-yl)(3-(6-(tetrahydro- | 2 | 420.517 | 421.35 |
| | 2H-pyran-4-ylamino)imidazo[1,2- | | | |
| | b]pyridazin-3-yl)phenyl)methanone | | | |
| 119 | (4-methylpiperazin-1-yl)(4-(6-(tetrahydro- | 2 | 420.517 | 421.28 |
| | 2H-pyran-4-ylamino)imidazo[1,2- | | | |
| | b]pyridazin-3-yl)phenyl)methanone | | | |
| 120 | (E)—N-(tetrahydro-2H-pyran-4-yl)-3-(2- | 2 | 316.481 | 317.33 |
| | (trimethylsilyl)vinyl)imidazo[1,2- | | | |
| | b]pyridazin-6-amine | | | |
| 121 | (4-(6-(tetrahydro-2H-pyran-4- | 2 | 324.384 | 325.31 |
| | ylamino)imidazo[1,2-b]pyridazin-3- | | | |
| | yl)phenyl)methanol | | | |
| 122 | 3-(3-(2-chlorobenzyloxy)phenyl)-N- | 2 | 434.927 | 435.28 |
| | (tetrahydro-2H-pyran-4-yl)imidazo[1,2- | | | |
| | b]pyridazin-6-amine | | | |
| 123 | (E)-3-(oct-1-enyl)-N-(tetrahydro-2H-pyran- | 2 | 328.46 | 329.36 |
| | 4-yl)imidazo[1,2-b]pyridazin-6-amine | | | |
| 124 | 4-(6-(tetrahydro-2H-pyran-4- | 2 | 337.383 | 338.25 |
| | ylamino)imidazo[1,2-b]pyridazin-3- | | | |
| | yl)benzamide | | | |
| 125 | 2-fluoro-4-(6-(tetrahydro-2H-pyran-4- | 2 | 370.388 | 371.26 |
| | ylamino)imidazo[1,2-b]pyridazin-3- | | | |
| | yl)benzohydrazide | | | |
| 126 | 2-(4-(6-(tetrahydro-2H-pyran-4- | 2 | 333.395 | 334.33 |
| | ylamino)imidazo[1,2-b]pyridazin-3- | | | |
| | yl)phenyl)acetonitrile | | | |
| 127 | N-(4-(6-(tetrahydro-2H-pyran-4- | 2 | 387.462 | 388.26 |
| | ylamino)imidazo[1,2-b]pyridazin-3- | | | |
| | yl)phenyl)methanesulfonamide | | | |
| 128 | (S)-2-amino-3-(4-(6-(tetrahydro-2H-pyran- | 2 | 381.436 | 382.32 |
| | 4-ylamino)imidazo[1,2-b]pyridazin-3- | | | |
| | yl)phenyl)propanoic acid | | | |
| 129 | (E)-3-(pent-1-enyl)-N-(tetrahydro-2H- | 2 | 286.379 | 287.33 |
| | pyran-4-yl)imidazo[1,2-b]pyridazin-6-amine | | | |
| 130 | 3-(4-methyl-3,4-dihydro-2H- | 2 | 365.437 | 366.3 |
| | benzo[b][1,4]oxazin-6-yl)-N-(tetrahydro- | | | |
| | 2H-pyran-4-yl)imidazo[1,2-b]pyridazin-6- | | | |
| | amine | | | |
| 131 | 3-(4-ethylphenyl)-N-(tetrahydro-2H-pyran- | 2 | 322.412 | 323.28 |
| | 4-yl)imidazo[1,2-b]pyridazin-6-amine | | | |
| 132 | (R)-2-amino-3-(4-(6-(tetrahydro-2H-pyran- | 2 | 381.436 | 382.25 |
| | 4-ylamino)imidazo[1,2-b]pyridazin-3- | | | |
| | yl)phenyl)propanoic acid | | | |
| 133 | 5-(6-(tetrahydro-2H-pyran-4- | 2 | 344.393 | 345.24 |
| | ylamino)imidazo[1,2-b]pyridazin-3- | | | |
| | yl)thiophene-2-carboxylic acid | | | |
| 134 | (E)-3-styryl-N-(tetrahydro-2H-pyran-4- | 2 | 320.396 | 321.32 |
| | yl)imidazo[1,2-b]pyridazin-6-amine | | | |
| 135 | 3-(5-chlorothiophen-2-yl)-N-(tetrahydro- | 2 | 334.829 | 335.17 |
| | 2H-pyran-4-yl)imidazo[1,2-b]pyridazin-6- | | | |
| | amine | | | |
| 136 | 3-(5-methylthiophen-2-yl)-N-(tetrahydro- | 2 | 314.411 | 315.24 |
| | 2H-pyran-4-yl)imidazo[1,2-b]pyridazin-6- | | | |
| | amine | | | |
| 137 | 3-(2,4-difluorophenyl)-N-(tetrahydro-2H- | 2 | 330.338 | 331.25 |
| | pyran-4-yl)imidazo[1,2-b]pyridazin-6-amine | | | |
| 138 | 3-(3,4-difluorophenyl)-N-(tetrahydro-2H- | 2 | 330.338 | 331.25 |
| | pyran-4-yl)imidazo[1,2-b]pyridazin-6-amine | | | |
| 139 | 3-(4-tert-butylphenyl)-N-(tetrahydro-2H- | 2 | 350.466 | 351.33 |
| | pyran-4-yl)imidazo[1,2-b]pyridazin-6-amine | | | |
| 140 | N-(2-hydroxyethyl)-4-(6-(tetrahydro-2H- | 2 | 417.488 | 418.28 |
| | pyran-4-ylamino)imidazo[1,2-b]pyridazin-3- | | | |
| | yl)benzenesulfonamide | | | |
| 141 | 4-(6-(tetrahydro-2H-pyran-4- | 2 | 405.422 | 406.24 |
| | ylamino)imidazo[1,2-b]pyridazin-3-yl)-N- | | | |
| | (1H-tetrazol-5-yl)benzamide | | | |
| 142 | 4-(6-(tetrahydro-2H-pyran-4- | 2 | 373.435 | 374.27 |
| | ylamino)imidazo[1,2-b]pyridazin-3- | | | |
| | yl)benzenesulfonamide | | | |

The ability of compounds of Formula (I) to modulate the activity of IRAK proteins can be assessed by the method described in the following example.

### Example 5

### IRAK4 TR-FRET Assay

Materials

Biotinylated IRAK1 peptide (IRAK1 AA358-389, GLARFSRFAGSSPSQSSMVARTQTVRGTLA [SEQ ID NO: 1]. N-terminus:Biotin, C-terminus:Amide) was synthesized by Advanced ChemTech (Louisville, Ky.), Streptavidin Allophycocyanin (SA-APC) was obtained from ProZyme (San Leandro, Calif.), Polyclonal AntiphosphoThreonine antibody was obtained from Cell Signaling Technologies, Inc. (Danvers, Mass.), LANCE Eu-W1024 Anti Rabbit IgG and LANCE 10× detection buffer were obtained from Perkin Elmer (Wellesley, Mass.), SuperBlok in TBS was obtained from Pierce (Rockford, Ill.), ATP was purchased from Invitrogen (Carlsbad, Calif.) and DMSO was obtained from Fisher. Scientific (Fairlawn N.J.).

The IRAK 4 Construct CH373 was synthesized at Biogen Idec Inc. Its amino acid sequence is

[SEQ ID NO: 2]
MSYYHHHHHHDYDIPTTENLYFQGAMGDRTLMTPVQNLEQSYMPPDSS
SPENKSLEVSDTRFHSFSFYELKNVTNNFDERPISVGGNKMGEGGFGV
VYKGYVNNTTVAVKKLAAMVDITTEELKQQFDQEIKVMAKCQHENLVE
LLGFSSDGDDLCLVYVYMPNGSLLDRLSCLDGTPPLSWHMRCKIAQGA
ANGINFLHENHHIHRDIKSANILLDEAFTAKISDFGLARASEKFAQTV
MTSRIVGTTAYMAPEALRGEITPKSDIYSFGVVLLEIITGLPAVDEHR
EPQLLLDIKEEIEDEEKTIEDYIDKKMNDADSTSVEAMYSVASQCLHE
KKNKRPDIKKVQQLLQEMTAS.

Assay

5 μL of a solution of the test compound at a concentration of 50 μM or less in 1% (v/v) DMSO was added to the wells of a 96-well ½ area Black Polystyrene plates (Costar 3694). The final concentrations in the reaction well were 10 μM ATP, 0.5 ηM IRAK4 CH373, 1.6 μM IRAK1 peptide, 1% DMSO, 50 mM HEPES, 60 mM NaCl, 1 mM MgCl₂, 2 mM DTT, 5 mM MnCl₂, 0.01% BSA, and 0.01% Tween-20. The volume of the reaction was 45 μL. The reaction mixture was incubated at room temperature for 30 minutes and stopped with the addition of 5 μL 100 mM EDTA.

Added to each well were 25 μL of a solution containing 160 ηM SA-APC, 1× LANCE detection buffer and 1% Superblock in TBS, and 25 μL of a solution containing 100 ηM Polyclonal Anti p-Thr, 20 ηM Eu-Anti Rabbit IgG, 1× LANCE detection buffer and 1% Superblock in TBS. The plates were covered with a foil lid and incubated for at least 30 minutes at room temperature. The plates were read on an Analyst AD, LJL BioSystems, ID 1615. The recommended settings were: Type: MultiMethod; Name: HTRF-EuK; Plate format: LJL HE 96 A Black PS; Z height: 2 mm; Raw units: counts; Ratio: acceptor/donor, Acceptor: HRTF(Packard) acceptor: Excitation: Europium FRET 330 ηm, Emission: FRET acceptor 665 ηm, Donor: HRTF(Packard) donor: Excitation: Europium FRET 330 ηm, Emission: FRET chelate donor; Flashes/well: 100; Intergration time: 400 μs; Interval between: 1×10 ms flashes; Delay after flash: 50 μs. Control wells measuring total signal contained 1% (v/v) DMSO only (no test compound). Control wells measuring background signal contained 1% (v/v) DMSO/50 mM EDTA.

Compounds of Formula (I) typically exhibited IC₅₀ values of less than 20 μM; some of the compounds exhibited IC₅₀ values of less than 1 μM; and some had IC₅₀ values of less than 10 nM.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of this invention.

1. A method of treating an inflammatory condition, a cell proliferative disorder, or an immune disorder, comprising administering to a subject in need of such treatment a therapeutically effective amount of a compound of Formula (I) 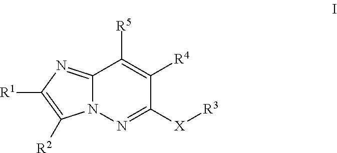
or a pharmaceutically acceptable salt thereof, wherein
each of R¹, R², R⁴, and R⁵ is independently H, halo, an optionally substituted amino, an optionally substituted aliphatic, an optionally substituted cycloaliphatic, an optionally substituted heterocycloaliphatic, an optionally substituted aryl, or an optionally substituted heteroaryl;
R³ is H, an optionally substituted aliphatic, an optionally substituted cycloaliphatic, an optionally substituted heterocycloaliphatic, an optionally substituted aryl, or an optionally substituted heteroaryl;
X is O, C(O), N(R) or S(O)ₙ;
n is 0, 1, or 2; and
R is H, an optionally substituted aliphatic, an optionally substituted cycloaliphatic, an optionally substituted heterocycloaliphatic, an optionally substituted aryl, an optionally substituted heteroaryl; or
when X is N(R), R³ and R, together with the nitrogen atom to which they are attached, may form a 3- to 7-membered optionally substituted heterocycloaliphatic or heteroaryl ring.

2. The method of claim 1, wherein R³ is an optionally substituted aliphatic.

3. The method of claim 2, wherein R³ is an aliphatic optionally substituted with an optionally substituted aryl, or an optionally substituted heteroaryl; or, when X is O, S, or N(R), R³ is an aliphatic substituted with an optionally substituted cycloaliphatic or an optionally substituted heterocycloaliphatic.

4. The method of claim 3, wherein the optionally substituted aryl or optionally substituted heteroaryl substituent on R³ is optionally substituted with amino, halo, hydroxy, alkoxy, sulfonamide, haloalkyl, cyano, nitro, an optionally substituted cycloaliphatic, or an optionally substituted heterocycloaliphatic; and wherein the cycloaliphatic or heterocycloaliphatic substituent on R³ is optionally substituted with halo, amino, hydroxy, oxo, alkoxy, alkyl, or sulfonamide.

5. The method of claim 4, wherein X is N(R) or O.

6. The method of claim 4, wherein R³X— is

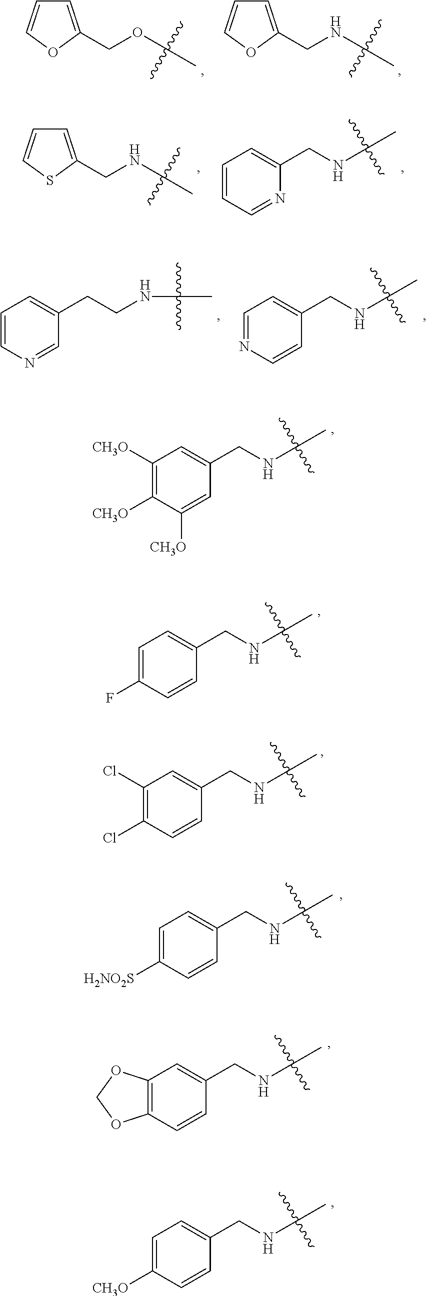

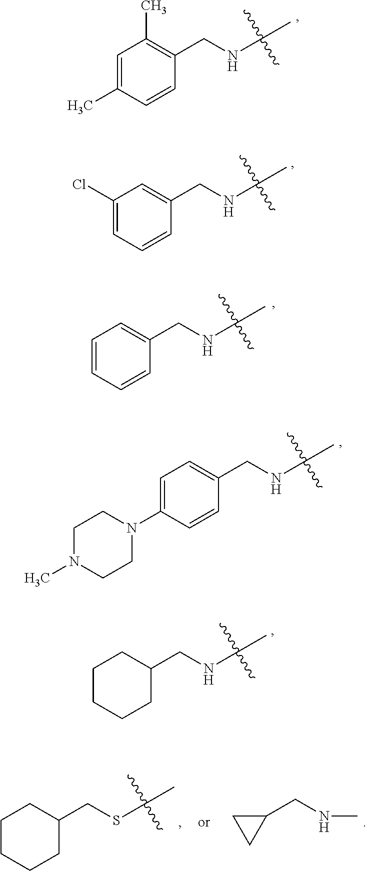

7. The method of claim 2, wherein R³ is an aliphatic optionally substituted with halo, amino, hydroxy, oxo, alkoxy, sulfonamide, or an optionally substituted heterocycloaliphatic.

8. The method of claim 7, wherein R³X— is

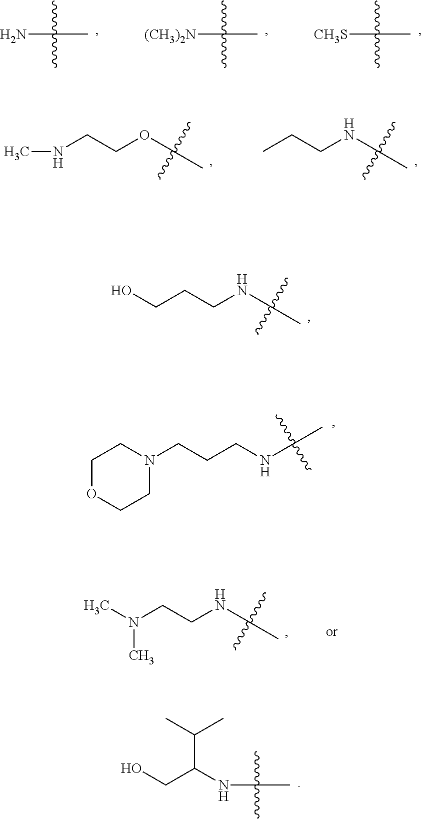

9. (canceled)

10. (canceled)

11. (canceled)

12. The method of claim 1, wherein R³ is an optionally substituted cycloaliphatic or an optionally substituted heterocycloaliphatic.

13. The method of claim 12, wherein R³ is a cycloalkyl or a heterocycloalkyl, and is optionally substituted with halo, hydroxy, oxo, alkoxy, alkyl, or sulfonamide.

14. The method of claim 13, wherein R³X— is

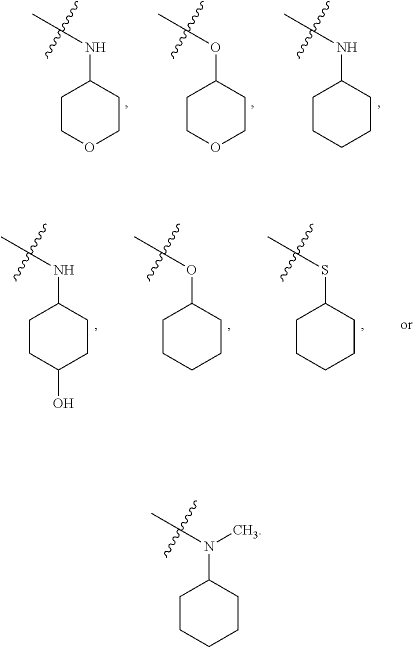

15. The method of claim 2, wherein n is 0.

16. The method of claim 1, wherein X is N(R); and R and R³, together with the nitrogen atom to which they are attached, form an optionally substituted heterocycloaliphatic or heteroaryl ring.

17. The method of claim 16, wherein the heterocycloaliphatic ring or heteroaryl ring is substituted with halo, amino, hydroxy, oxo, alkoxy, alkyl, or sulfonamide.

18. The method of claim 17, wherein R³X— is

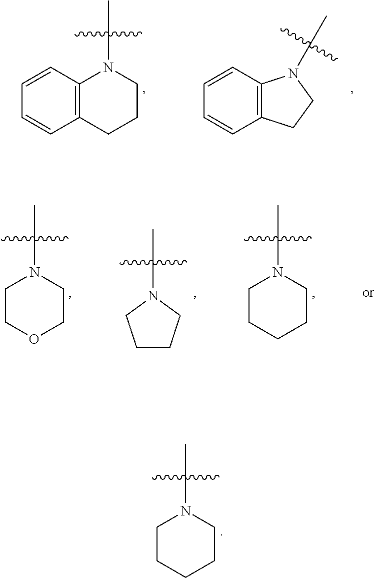

19. The method of claim 1, wherein R³ is an optionally substituted aryl.

20. The method of claim 19, wherein R³ is phenyl optionally substituted with cyano, halo, haloalkyl, amino, hydroxy, alkoxy, alkoxycarbonyl, amido, alkyl, alkylcarbonylalkyl, sulfonamide, cycloaliphatic, or heterocycloaliphatic.

21. The method of claim 20, wherein R³X— is

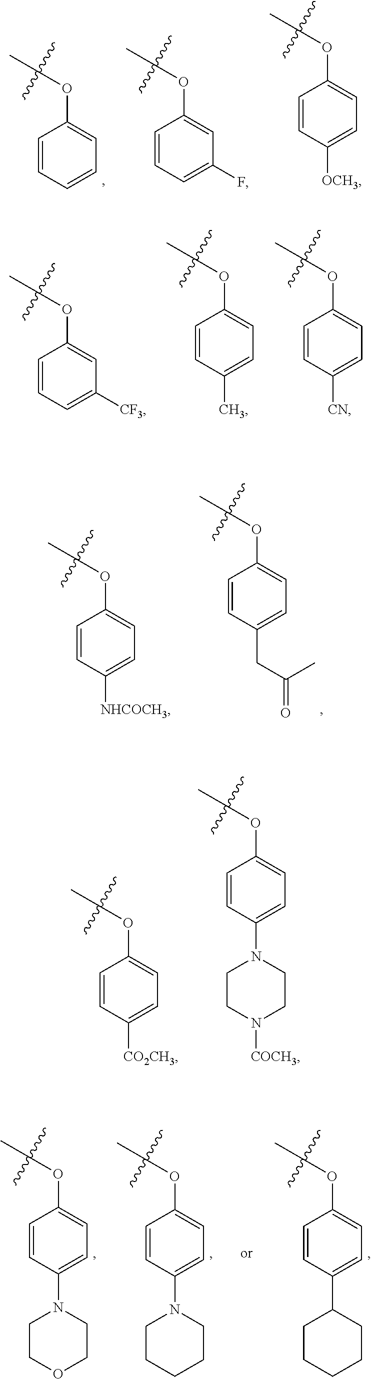

22. (canceled)

23. The method of claim 1, wherein R² is H, halo, or amino.

24. The method of claim 1, wherein R² is an optionally substituted aryl or an optionally substituted heteroaryl.

25. The method of claim 24, wherein R² is phenyl or napthyl, optionally substituted with 1 to 3 substituents each independently selected from the group consisting of halo, cyano, nitro, hydroxy, alkoxy, alkoxy-alkoxy, haloalkoxy, haloalkyl, alkylsulfanyl, alkyl, alkenyl, alkynyl, silylalkenyl, alkylcarbonylalkyl, or carboxy; or R² is pyrimidinyl, pyridinyl, indolyl, thiophenyl, quinoxalinyl, benzo-oxadiazole, pyrrolyl, triazolyl, tetrazolyl, indazolyl, benzofuranyl, dihydrobenzo-oxazine, furanyl, benzothiophenyl, quinolinyl, or pyrazolyl, optionally substituted with halo, cyano, alkyl, aralkyl, acyl, alkoxy, hydroxyalkyl, alkoxyalkyl, or carboxy.

26. The method of claim 25, wherein R² is

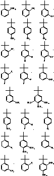

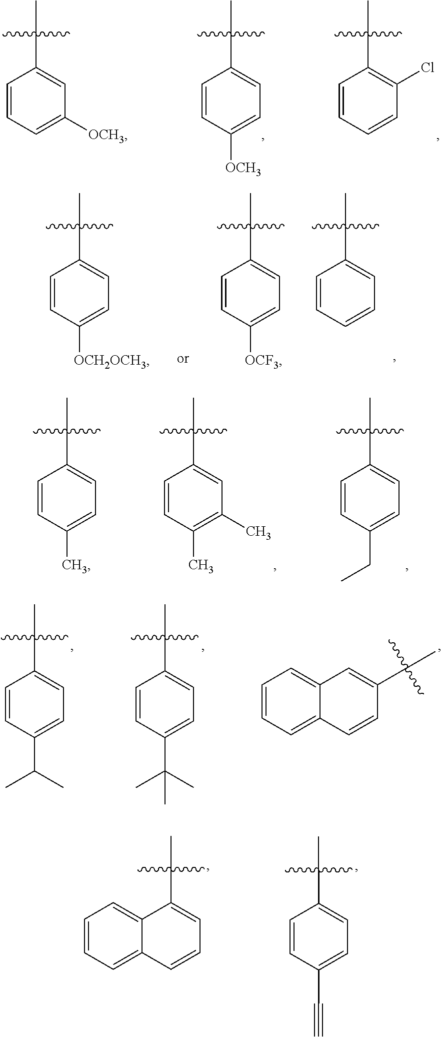

27. (canceled)

28. (canceled)

29. The method of claim 1, wherein R² is an optionally substituted heteroaryl.

30. The method of claim 29, wherein R² is pyrimidinyl, pyridinyl, indolyl, thiophenyl, quinoxalinyl, benzo-oxadiazole, pyrrolyl, triazolyl, tetrazolyl, indazolyl, benzofuranyl, dihydrobenzo-oxazine, furanyl, benzothiophenyl, quinolinyl, or pyrazolyl; and is optionally substituted with halo, cyano, alkyl, aralkyl, acyl, alkoxy, hydroxyalkyl, alkoxyalkyl, or carboxy.

31. (canceled)

32. The method of claim 1, wherein R² is an optionally substituted alkyl, an optionally substituted alkenyl, an optionally substituted alkynyl, an optionally substituted heterocycloalkenyl, or an optionally substituted cycloalkenyl.

33. The method of claim 32, wherein R² is

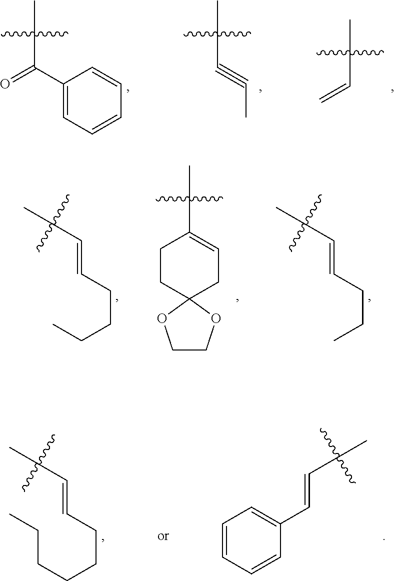

34. The method of claim 1, wherein the compound is
N-(2-hydroxyethyl)-3-(6-(thiophen-2-ylmethylamino)imidazo[1,2-b]pyridazin-3-yl)benzamide;
N-(furan-2-ylmethyl)-3-(5-isopropyl-2-methoxyphenyl)imidazo[1,2-b]pyridazin-6-amine;
3-(6-(furan-2-ylmethylamino)imidazo[1,2-b]pyridazin-3-yl)benzoic acid;
N-(pyridin-2-ylmethyl)-3-(4-(trifluoromethoxy)phenyl)imidazo[1,2-b]pyridazin-6-amine;
3-(thiophen-2-yl)-N-(thiophen-2-ylmethyl)imidazo[1,2-b]pyridazin-6-amine;
N-(3-(6-(thiophen-2-ylmethylamino)imidazo[1,2-b]pyridazin-3-yl)phenyl)acetamide;
3-(4-aminophenyl)-N-(thiophen-2-ylmethyl)imidazo[1,2-b]pyridazin-6-amine;
4-(6-(furan-2-ylmethylamino)imidazo[1,2-b]pyridazin-3-yl)phenol;
2-methoxy-4-(6-(thiophen-2-ylmethylamino)imidazo[1,2-b]pyridazin-3-yl)phenol;
2-(3-(5-isopropyl-2-methoxyphenyl)imidazo[1,2-b]pyridazin-6-ylamino)-3-methylbutan-1-ol;
N-(2-methoxyethyl)-3-(naphthalen-2-yl)imidazo[1,2-b]pyridazin-6-amine;
3-(3-aminophenyl)-N-(tetrahydro-2H-pyran-4-yl)imidazo[1,2-b]pyridazin-6-amine;
3-(benzo[d][1;3]dioxol-5-yl)-N-(pyridin-2-ylmethyl)imidazo[1,2-b]pyridazin-6-amine;
N-(2-methoxyethyl)-3-(quinolin-8-yl)imidazo[1,2-b]pyridazin-6-amine;
N-(3-(6-(2-methoxyethylamino)imidazo[1,2-b]pyridazin-3-yl)phenyl)acetamide;
N-(3-(6-(tetrahydro-2H-pyran-4-ylamino)imidazo[1,2-b]pyridazin-3-yl)phenyl)acetamide;
4-((3-(3,4-dimethoxyphenyl)imidazo[1,2-b]pyridazin-6-ylamino)methyl)-benzenesulfonamide;
4-((3-(4-(trifluoromethoxy)phenyl)imidazo[1,2-b]pyridazin-6-ylamino)methyl)benzenesulfonamide;
N-(tetrahydro-2H-pyran-4-yl)-3-(3,4,5-trimethoxyphenyl)imidazo[1,2-b]pyridazin-6-amine;
3-(3-(dimethylamino)phenyl)-N-(tetrahydro-2H-pyran-4-yl)imidazo[1,2-b]pyridazin-6-amine;
(E)-3-(3-(6-(3-hydroxypropylamino)imidazo[1,2-b]pyridazin-3-yl)phenyl)acrylic acid;
3-(3-(1-benzyl-1H-pyrazol-4-yl)imidazo[1,2-b]pyridazin-6-ylamino)propan-1-ol;
N-(tetrahydro-2H-pyran-4-yl)-3-(3-(trifluoromethoxy)phenyl)imidazo[1,2-b]pyridazin-6-amine;
4-((3-(4-(hydroxymethyl)phenyl)imidazo[1,2-b]pyridazin-6-ylamino)methyl)benzenesulfonamide;
4-(6-(3-hydroxypropylamino)imidazo[1,2-b]pyridazin-3-yl)-2-methoxyphenol;
(E)-3-(3-(6-(2-methoxyethylamino)imidazo[1,2-b]pyridazin-3-yl)phenyl)acrylic acid;
4-((3-(6-methoxypyridin-3-yl)imidazo[1,2-b]pyridazin-6-ylamino)methyl)benzenesulfonamide;
3-(6-(3,4,5-trimethoxybenzylamino)imidazo[1,2-b]pyridazin-3-yl)phenol;
4-((3-(4-hydroxy-3-methoxyphenyl)imidazo[1,2-b]pyridazin-6-ylamino)methyl)benzenesulfonamide;
3-(5-methoxypyridin-3-yl)-N-(tetrahydro-2H-pyran-4-yl)imidazo[1,2-b]pyridazin-6-amine;
3-(6-(2-methoxyethylamino)imidazo[1,2-b]pyridazin-3-yl)benzaldehyde;
(E)-3-(3-(hex-1-enyl)phenyl)-N-(tetrahydro-2H-pyran-4-yl)imidazo[1,2-b]pyridazin-6-amine;
4-((3-(3-formylphenyl)imidazo[1,2-b]pyridazin-6-ylamino)methyl)benzenesulfonamide;
3-(5-isopropyl-2-methoxyphenyl)-N-(tetrahydro-2H-pyran-4-yl)imidazo[1,2-b]pyridazin-6-amine;
1-(3-(6-(2-methoxyethylamino)imidazo[1,2-b]pyridazin-3-yl)phenyl)ethanone;
N-(2-methoxyethyl)-3-(4-morpholinophenyl)imidazo[1,2-b]pyridazin-6-amine;
N-(benzo[d][1,3]dioxol-5-ylmethyl)-3-(pyridin-4-yl)imidazo[1,2-b]pyridazin-6-amine;
3-(4-(dimethylamino)phenyl)-N-(tetrahydro-2H-pyran-4-yl)imidazo[1,2-b]pyridazin-6-amine;
3-phenyl-N-(tetrahydro-2H-pyran-4-yl)imidazo[1,2-b]pyridazin-6-amine;
3-(naphthalen-1-yl)-N-(tetrahydro-2H-pyran-4-yl)imidazo[1,2-b]pyridazin-6-amine;
3-(2-phenoxyphenyl)-N-(tetrahydro-2H-pyran-4-yl)imidazo[1,2-b]pyridazin-6-amine;
3-(benzo[d][1,3]dioxol-5-yl)-N-(tetrahydro-2H-pyran-4-yl)imidazo[1,2-b]pyridazin-6-amine;
3-(3-(3-aminophenyl)imidazo[1,2-b]pyridazin-6-ylamino)propan-1-ol;
3-(3-(benzo[d][1,3]dioxol-5-yl)imidazo[1,2-b]pyridazin-6-ylamino)propan-1-ol;
4-(6-(3-hydroxypropylamino)imidazo[1,2-b]pyridazin-3-yl)phenol;
N-(benzo[d][1,3]dioxol-5-ylmethyl)-3-(pyridin-3-yl)imidazo[1,2-b]pyridazin-6-amine;
3-(pyridin-3-yl)-N-(tetrahydro-2H-pyran-4-yl)imidazo[1,2-b]pyridazin-6-amine;
(E)-3-styryl-N-(tetrahydro-2H-pyran-4-yl)imidazo[1,2-b]pyridazin-6-amine;
3-(3,4-dimethoxyphenyl)-N-(tetrahydro-2H-pyran-4-yl)imidazo[1,2-b]pyridazin-6-amine;
4-(6-(tetrahydro-2H-pyran-4-ylamino)imidazo[1,2-b]pyridazin-3-yl)phenol;
3-(4-(6-(tetrahydro-2H-pyran-4-ylamino)imidazo[1,2-b]pyridazin-3-yl)phenyl)propanoic acid;
3-(2-methoxyphenyl)-N-(tetrahydro-2H-pyran-4-yl)imidazo[1,2-b]pyridazin-6-amine;
4-(6-(4-methoxybenzylamino)imidazo[1,2-b]pyridazin-3-yl)phenol;
3-(3-(3,4-dimethoxyphenyl)imidazo[1,2-b]pyridazin-6-ylamino)propan-1-ol;
4-((3-(4-hydroxyphenyl)imidazo[1,2-b]pyridazin-6-ylamino)methyl)benzenesulfonamide;
4-(6-(tetrahydro-2H-pyran-4-ylamino)imidazo[1,2-b]pyridazin-3-yl)benzonitrile;
4-(6-(tetrahydro-2H-pyran-4-ylamino)imidazo[1,2-b]pyridazin-3-yl)benzoic acid;
3-(furan-2-yl)-N-(tetrahydro-2H-pyran-4-yl)imidazo[1,2-b]pyridazin-6-amine;
3-(4-chlorophenyl)-N-(tetrahydro-2H-pyran-4-yl)imidazo[1,2-b]pyridazin-6-amine;
3-(3-chloro-4-fluorophenyl)-N-(tetrahydro-2H-pyran-4-yl)imidazo[1,2-b]pyridazin-6-amine;
3-(3,4-dimethylphenyl)-N-(tetrahydro-2H-pyran-4-yl)imidazo[1,2-b]pyridazin-6-amine;
3-(3-(3-(dimethylamino)phenyl)imidazo[1,2-b]pyridazin-6-ylamino)propan-1-ol;
(E)-3-(3-(hex-1-enyl)phenyl)-N-(3-methoxypropyl)imidazo[1,2-b]pyridazin-6-amine;
N-(benzo[d][1,3]dioxol-5-ylmethyl)-3-(5-methoxypyridin-3-yl)imidazo[1,2-b]pyridazin-6-amine;
N-(4-methoxyphenyl)-4-(6-(tetrahydro-2H-pyran-4-ylamino)imidazo[1,2-b]pyridazin-3-yl)benzamide;
3-(1H-indol-5-yl)-N-(tetrahydro-2H-pyran-4-yl)imidazo[1,2-b]pyridazin-6-amine;
3-(3-bromophenyl)-N-(tetrahydro-2H-pyran-4-yl)imidazo[1,2-b]pyridazin-6-amine;
3-(3-chlorophenyl)-N-(tetrahydro-2H-pyran-4-yl)imidazo[1,2-b]pyridazin-6-amine;
3-(4-methoxyphenyl)-N-(tetrahydro-2H-pyran-4-yl)imidazo[1,2-b]pyridazin-6-amine;
3-(6-(tetrahydro-2H-pyran-4-ylamino)imidazo[1,2-b]pyridazin-3-yl)phenol;
2-methoxy-4-(6-(2-methoxyethylamino)imidazo[1,2-b]pyridazin-3-yl)phenol;
2-methoxy-4-(6-(3,4,5-trimethoxybenzylamino)imidazo[1,2-b]pyridazin-3-yl)phenol;
N-(benzo[d][1,3]dioxol-5-ylmethyl)-3-(6-methoxypyridin-3-yl)imidazo[1,2-b]pyridazin-6-amine;
4-(6-(benzo[d][1,3]dioxol-5-ylmethylamino)imidazo[1,2-b]pyridazin-3-yl)-2-methoxyphenol;
(2-(6-(tetrahydro-2H-pyran-4-ylamino)imidazo[1,2-b]pyridazin-3-yl)phenyl)methanol;
3-(6-methoxypyridin-3-yl)-N-(tetrahydro-2H-pyran-4-yl)imidazo[1,2-b]pyridazin-6-amine;
N-(tetrahydro-2H-pyran-4-yl)-3-(3-(trifluoromethyl)phenyl)imidazo[1,2-b]pyridazin-6-amine;
3-(3-fluorophenyl)-N-(tetrahydro-2H-pyran-4-yl)imidazo[1,2-b]pyridazin-6-amine;
3-(4-aminophenyl)-N-(tetrahydro-2H-pyran-4-yl)imidazo[1,2-b]pyridazin-6-amine;
3-(4-fluorophenyl)-N-(tetrahydro-2H-pyran-4-yl)imidazo[1,2-b]pyridazin-6-amine;
3-(furan-3-yl)-N-(tetrahydro-2H-pyran-4-yl)imidazo[1,2-b]pyridazin-6-amine;
2-methoxy-4-(6-(4-methoxybenzylamino)imidazo[1,2-b]pyridazin-3-yl)phenol;
3-(4-aminophenyl)-N-(4-methoxybenzyl)imidazo[1,2-b]pyridazin-6-amine;
3-(4-phenoxyphenyl)-N-(tetrahydro-2H-pyran-4-yl)imidazo[1,2-b]pyridazin-6-amine;
3-(pyrimidin-5-yl)-N-(tetrahydro-2H-pyran-4-yl)imidazo[1,2-b]pyridazin-6-amine;
methyl 4-(6-(tetrahydro-2H-pyran-4-ylamino)imidazo[1,2-b]pyridazin-3-yl)benzoate;
3-(2-chlorophenyl)-N-(tetrahydro-2H-pyran-4-yl)imidazo[1,2-b]pyridazin-6-amine;
3-(2-fluorophenyl)-N-(tetrahydro-2H-pyran-4-yl)imidazo[1,2-b]pyridazin-6-amine;
N-(tetrahydro-2H-pyran-4-yl)-3-p-tolylimidazo[1,2-b]pyridazin-6-amine;
3-(4-methoxyphenyl)-N-(tetrahydro-2H-pyran-4-yl)imidazo[1,2-b]pyridazin-6-amine;
3-(3,5-dimethoxyphenyl)-N-(tetrahydro-2H-pyran-4-yl)imidazo[1,2-b]pyridazin-6-amine;
1-(3-(6-(tetrahydro-2H-pyran-4-ylamino)imidazo[1,2-b]pyridazin-3-yl)phenyl)ethanone;
N-(tetrahydro-2H-pyran-4-yl)-3-(thiophen-2-yl)imidazo[1,2-b]pyridazin-6-amine;
4-((3-(1-methyl-1H-pyrazol-4-yl)imidazo[1,2-b]pyridazin-6-ylamino)methyl)benzenesulfonamide;
4-((3-(1-benzyl-1H-pyrazol-4-yl)imidazo[1,2-b]pyridazin-6-ylamino)methyl)benzenesulfonamide;
3-(naphthalen-2-yl)-N-(2-(pyridin-3-yl)ethyl)imidazo[1,2-b]pyridazin-6-amine;
3-(naphthalen-2-yl)-N-(pyridin-4-ylmethyl)imidazo[1,2-b]pyridazin-6-amine;
3-(3,4-dimethoxyphenyl)-N-(furan-2-ylmethyl)imidazo[1,2-b]pyridazin-6-amine;
N-(furan-2-ylmethyl)-3-(4-(trifluoromethoxy)phenyl)imidazo[1,2-b]pyridazin-6-amine;
4-(6-(thiophen-2-ylmethylamino)imidazo[1,2-b]pyridazin-3-yl)phenol;
(R)-N-(3-(6-(1-hydroxy-3-methylbutan-2-ylamino)imidazo[1,2-b]pyridazin-3-yl)-phenyl)acetamide;
N-(furan-2-ylmethyl)-3-(4-methoxyphenyl)imidazo[1,2-b]pyridazin-6-amine;
(4-(6-(furan-2-ylmethylamino)imidazo[1,2-b]pyridazin-3-yl)phenyl)methanol;
4-(6-(cyclopropylmethylamino)imidazo[1,2-b]pyridazin-3-yl)-2-methoxyphenol;
4-(6-(furan-2-ylmethylamino)imidazo[1,2-b]pyridazin-3-yl)-2-methoxyphenol;
(R)-4-(6-(1-hydroxy-3-methylbutan-2-ylamino)imidazo[1,2-b]pyridazin-3-yl)-2-methoxyphenol;
N-(furan-2-ylmethyl)-3-(4-phenoxyphenyl)imidazo[1,2-b]pyridazin-6-amine;
3-(benzofuran-2-yl)-N-(3-chlorobenzyl)imidazo[1,2-b]pyridazin-6-amine;
4-(6-(3-chlorobenzylamino)imidazo[1,2-b]pyridazin-3-yl)phenol;
4-(6-(4-fluorobenzylamino)imidazo[1,2-b]pyridazin-3-yl)phenol;
N-(4-(4-methylpiperazin-1-yl)benzyl)-3-(4-(trifluoromethoxy)phenyl)imidazo[1,2-b]pyridazin-6-amine;
3-(6-(4-(4-methylpiperazin-1-yl)benzylamino)imidazo[1,2-b]pyridazin-3-yl)phenol;
4-(6-(3-chlorobenzylamino)imidazo[1,2-b]pyridazin-3-yl)-2-methoxyphenol;
2-methoxy-4-(6-(propylamino)imidazo[1,2-b]pyridazin-3-yl)phenol;
4-(6-(3,4-dichlorobenzylamino)imidazo[1,2-b]pyridazin-3-yl)-2-methoxyphenol;
4-(6-(2,4-dimethylbenzylamino)imidazo[1,2-b]pyridazin-3-yl)-2-methoxyphenol;
4-(6-(3-chlorobenzylamino)imidazo[1,2-b]pyridazin-3-yl)-N-(2-(dimethylamino)ethyl)benzamide;
N-(3-morpholinopropyl)-3-(naphthalen-2-yl)imidazo[1,2-b]pyridazin-6-amine;
N¹,N¹-dimethyl-N³-(3-(naphthalen-2-yl)imidazo[1,2-b]pyridazin-6-yl)propane-1,3-diamine; or
N-(3-(6-(furan-2-ylmethylamino)imidazo[1,2-b]pyridazin-3-yl)phenyl)methanesulfonamide.

35. A compound which is
6-(tetrahydro-2H-pyran-4-yloxy)imidazo[1,2-b]pyridazine;
6-(furan-2-ylmethoxy)imidazo[1,2-b]pyridazine;
N-cyclohexylimidazo[1,2-b]pyridazin-6-amine;
3-bromo-N-cyclohexylimidazo[1,2-b]pyridazin-6-amine;
3-(imidazo[1,2-b]pyridazin-6-ylamino)propan-1-ol;
3-bromo-6-(tetrahydro-2H-pyran-4-yloxy)imidazo[1,2-b]pyridazine;
3-bromo-6-(furan-2-ylmethoxy)imidazo[1,2-b]pyridazine;
4-(3-bromoimidazo[1,2-b]pyridazin-6-ylamino)cyclohexanol;
1-(3-(6-(cyclohexylamino)imidazo[1,2-b]pyridazin-3-yl)phenyl)-2-hydroxyethanone;
(3-(6-(cyclohexylamino)imidazo[1,2-b]pyridazin-3-yl)phenyl)methanol;
3-(6-(cyclohexylamino)imidazo[1,2-b]pyridazin-3-yl)benzoic acid;
4-(6-(furan-2-ylmethoxy)imidazo[1,2-b]pyridazin-3-yl)phenol;
4-(6-(tetrahydro-2H-pyran-4-yloxy)imidazo[1,2-b]pyridazin-3-yl)phenol;
2-hydroxy-1-(3-(6-(tetrahydro-2H-pyran-4-yloxy)imidazo[1,2-b]pyridazin-3-yl)phenyl)ethanone;
N-(3-(6-(tetrahydro-2H-pyran-4-ylamino)imidazo[1,2-b]pyridazin-3-yl)phenyl)acetamide;
3-(5-methoxypyridin-3-yl)-N-(tetrahydro-2H-pyran-4-yl)imidazo[1,2-b]pyridazin-6-amine;
(E)-3-(hex-1-enyl)-N-(tetrahydro-2H-pyran-4-yl)imidazo[1,2-b]pyridazin-6-amine;
4-(6-(3-hydroxypropylamino)imidazo[1,2-b]pyridazin-3-yl)-2-methoxyphenol;
4-(6-(furan-2-ylmethylamino)imidazo[1,2-b]pyridazin-3-yl)phenol;
2-hydroxy-1-(3-(6-(3-hydroxypropylamino)imidazo[1,2-b]pyridazin-3-yl)phenyl)ethanone;
4-(6-(3-hydroxypropylamino)imidazo[1,2-b]pyridazin-3-yl)phenol;
4-(6-(4-hydroxycyclohexylamino)imidazo[1,2-b]pyridazin-3-yl)phenol;
4-(3-(3-(hydroxymethyl)phenyl)imidazo[1,2-b]pyridazin-6-ylamino)cyclohexanol;
3-(6-(4-hydroxycyclohexylamino)imidazo[1,2-b]pyridazin-3-yl)benzoic acid;
4-(6-(isopropylamino)imidazo[1,2-b]pyridazin-3-yl)phenol;
4-(6-(tetrahydro-2H-pyran-4-ylamino)imidazo[1,2-b]pyridazin-3-yl)phenol;
3-(6-(3-hydroxypropylamino)imidazo[1,2-b]pyridazin-3-yl)benzoic acid;
3-(6-(cyclohexylamino)imidazo[1,2-b]pyridazin-3-yl)benzamide;
3-(3-(3-(hydroxymethyl)phenyl)imidazo[1,2-b]pyridazin-6-ylamino)propan-1-ol;
4-(6-(tetrahydro-2H-pyran-4-ylamino)imidazo[1,2-b]pyridazin-3-yl)benzonitrile;
3-(6-(3-hydroxypropylamino)imidazo[1,2-b]pyridazin-3-yl)benzamide;
3-bromo-N-(tetrahydro-2H-pyran-4-yl)imidazo[1,2-b]pyridazin-6-amine;
N-cyclohexyl-3-(4-fluorophenyl)imidazo[1,2-b]pyridazin-6-amine;
4-(6-(cyclohexylamino)imidazo[1,2-b]pyridazin-3-yl)benzonitrile;
3-(4-fluorophenyl)-N-(tetrahydro-2H-pyran-4-yl)imidazo[1,2-b]pyridazin-6-amine;
N-cyclohexyl-3-(4-methoxyphenyl)imidazo[1,2-b]pyridazin-6-amine;
1-(3-(6-(tetrahydro-2H-pyran-4-ylamino)imidazo[1,2-b]pyridazin-3-yl)phenyl)ethanone;
3-(4-(methoxymethoxy)phenyl)-N-(tetrahydro-2H-pyran-4-yl)imidazo[1,2-b]pyridazin-6-amine;
3-(4-(dimethylamino)phenyl)-N-(tetrahydro-2H-pyran-4-yl)imidazo[1,2-b]pyridazin-6-amine;
4-(6-(tetrahydro-2H-pyran-4-ylamino)imidazo[1,2-b]pyridazin-3-yl)benzaldehyde;
3-(3,4-dimethoxyphenyl)-N-(tetrahydro-2H-pyran-4-yl)imidazo[1,2-b]pyridazin-6-amine;
3-(2-methoxypyrimidin-5-yl)-N-(tetrahydro-2H-pyran-4-yl)imidazo[1,2-b]pyridazin-6-amine;
4-(6-(cyclohexylamino)imidazo[1,2-b]pyridazin-3-yl)phenol;
(S)-4-(6-(1-hydroxy-3-methylbutan-2-ylamino)imidazo[1,2-b]pyridazin-3-yl)-2-methoxyphenol;
3-(4-methoxypyridin-3-yl)-N-(tetrahydro-2H-pyran-4-yl)imidazo[1,2-b]pyridazin-6-amine;
3-(1-methyl-1H-indol-5-yl)-N-(tetrahydro-2H-pyran-4-yl)imidazo[1,2-b]pyridazin-6-amine;
tert-butyl 2-(6-(tetrahydro-2H-pyran-4-ylamino)imidazo[1,2-b]pyridazin-3-yl)-1H-indole-1-carboxylate;
3-(1H-indol-2-yl)-N-(tetrahydro-2H-pyran-4-yl)imidazo[1,2-b]pyridazin-6-amine;
3-(6-(tetrahydro-2H-pyran-4-ylamino)imidazo[1,2-b]pyridazin-3-yl)benzonitrile;
3-(4-(methylsulfonyl)phenyl)-N-(tetrahydro-2H-pyran-4-yl)imidazo[1,2-b]pyridazin-6-amine;
N-(tetrahydro-2H-pyran-4-yl)-3-(4-vinylphenyl)imidazo[1,2-b]pyridazin-6-amine;
3-(4-ethynylphenyl)-N-(tetrahydro-2H-pyran-4-yl)imidazo[1,2-b]pyridazin-6-amine;
3-(2-methoxyphenyl)-N-(tetrahydro-2H-pyran-4-yl)imidazo[1,2-b]pyridazin-6-amine;
2-(6-(tetrahydro-2H-pyran-4-ylamino)imidazo[1,2-b]pyridazin-3-yl)phenol;
2-methoxy-4-(6-(tetrahydro-2H-pyran-4-ylamino)imidazo[1,2-b]pyridazin-3-yl)phenol;
(S)-2-(3-(4-fluorophenyl)imidazo[1,2-b]pyridazin-6-ylamino)-3-methylbutan-1-ol;
(S)-4-(6-(1-hydroxy-3-methylbutan-2-ylamino)imidazo[1,2-b]pyridazin-3-yl)benzonitrile;
N-(tetrahydro-2H-pyran-4-yl)-3-vinylimidazo[1,2-b]pyridazin-6-amine;
(S)-4-(6-(1-hydroxy-3-methylbutan-2-ylamino)imidazo[1,2-b]pyridazin-3-yl)-2-methoxyphenol;
(S)-4-(6-(1-hydroxy-3-methylbutan-2-ylamino)imidazo[1,2-b]pyridazin-3-yl)benzaldehyde;
(S)-4-(6-(1-hydroxy-3-methylbutan-2-ylamino)imidazo[1,2-b]pyridazin-3-yl)phenol;
3-(6-fluoropyridin-3-yl)-N-(tetrahydro-2H-pyran-4-yl)imidazo[1,2-b]pyridazin-6-amine;
N-(tetrahydro-2H-pyran-4-yl)-3-(4-(trifluoromethyl)phenyl)imidazo[1,2-b]pyridazin-6-amine;
2-(6-(tetrahydro-2H-pyran-4-ylamino)imidazo[1,2-b]pyridazin-3-yl)benzonitrile;
3-(4-nitrophenyl)-N-(tetrahydro-2H-pyran-4-yl)imidazo[1,2-b]pyridazin-6-amine;
4-oxo-4-(4-(6-(tetrahydro-2H-pyran-4-ylamino)imidazo[1,2-b]pyridazin-3-yl)-phenylamino)butanoic acid;
N-(4-(6-(tetrahydro-2H-pyran-4-ylamino)imidazo[1,2-b]pyridazin-3-yl)phenyl)acetamide;
N-(tetrahydro-2H-pyran-4-yl)-3-(thiophen-3-yl)imidazo[1,2-b]pyridazin-6-amine;
3-(4-(methylthio)phenyl)-N-(tetrahydro-2H-pyran-4-yl)imidazo[1,2-b]pyridazin-6-amine;
2-(4-(6-(tetrahydro-2H-pyran-4-ylamino)imidazo[1,2-b]pyridazin-3-yl)phenyl)acetonitrile;
3-(4-(aminomethyl)phenyl)-N-(tetrahydro-2H-pyran-4-yl)imidazo[1,2-b]pyridazin-6-amine;
N-methyl-3-(6-(tetrahydro-2H-pyran-4-ylamino)imidazo[1,2-b]pyridazin-3-yl)benzamide;
3-(quinoxalin-6-yl)-N-(tetrahydro-2H-pyran-4-yl)imidazo[1,2-b]pyridazin-6-amine;
1-(5-(6-(tetrahydro-2H-pyran-4-ylamino)imidazo[1,2-b]pyridazin-3-yl)thiophen-2-yl)ethanone;
2-fluoro-5-(6-(tetrahydro-2H-pyran-4-ylamino)imidazo[1,2-b]pyridazin-3-yl)benzonitrile;
2-fluoro-5-(6-(tetrahydro-2H-pyran-4-ylamino)imidazo[1,2-b]pyridazin-3-yl)benzaldehyde;
3-(3,4-dichlorophenyl)-N-(tetrahydro-2H-pyran-4-yl)imidazo[1,2-b]pyridazin-6-amine;
(5-(6-(tetrahydro-2H-pyran-4-ylamino)imidazo[1,2-b]pyridazin-3-yl)thiophen-2-yl)methanol;
2-(4-(6-(tetrahydro-2H-pyran-4-ylamino)imidazo[1,2-b]pyridazin-3-yl)benzyl)isoindoline-1;3-dione;
piperidin-1-yl(4-(6-(tetrahydro-2H-pyran-4-ylamino)imidazo[1,2-b]pyridazin-3-yl)phenyl)methanone;
3-(3-(piperidin-1-yl)phenyl)-N-(tetrahydro-2H-pyran-4-yl)imidazo[1,2-b]pyridazin-6-amine;
3-(4-(morpholinomethyl)phenyl)-N-(tetrahydro-2H-pyran-4-yl)imidazo[1,2-b]pyridazin-6-amine;
N-(4-(6-(tetrahydro-2H-pyran-4-ylamino)imidazo[1,2-b]pyridazin-3-yl)benzyl)methanesulfonamide;
3-(benzo[c][1,2;5]oxadiazol-5-yl)-N-(tetrahydro-2H-pyran-4-yl)imidazo[1,2-b]pyridazin-6-amine;
4-(3-(4-fluorophenyl)imidazo[1,2-b]pyridazin-6-yl)morpholine;
N-cyclohexyl-3-(4-fluorophenyl)-N-methylimidazo[1,2-b]pyridazin-6-amine;
3-(3-(4-fluorophenyl)imidazo[1,2-b]pyridazin-6-ylamino)propan-1-ol;
2-(3-(4-fluorophenyl)imidazo[1,2-b]pyridazin-6-ylamino)ethanol;
(S)-1-(5-(6-(1-hydroxy-3-methylbutan-2-ylamino)imidazo[1,2-b]pyridazin-3-yl)thiophen-2-yl)ethanone;
N-benzyl-3-(4-fluorophenyl)imidazo[1,2-b]pyridazin-6-amine;
N-(cyclohexylmethyl)-3-(4-fluorophenyl)imidazo[1,2-b]pyridazin-6-amine;
4-(6-(cyclohexylthio)imidazo[1,2-b]pyridazin-3-yl)benzonitrile;
6-(cyclohexyloxy)-3-(4-fluorophenyl)imidazo[1,2-b]pyridazine;
4-(6-(cyclohexyloxy)imidazo[1,2-b]pyridazin-3-yl)benzonitrile;
4-(6-(tetrahydro-2H-pyran-4-yloxy)imidazo[1,2-b]pyridazin-3-yl)benzonitrile;
3-(4-fluorophenyl)-6-(tetrahydro-2H-pyran-4-yloxy)imidazo[1,2-b]pyridazine;
N-(3-bromo-6-(tetrahydro-2H-pyran-4-yloxy)imidazo[1,2-b]pyridazin-2-yl)-2,2,2-trifluoroacetamide;
3-(4-fluorophenyl)-6-(tetrahydro-2H-pyran-4-yloxy)imidazo[1,2-b]pyridazin-2-amine;
4-(2-amino-6-(tetrahydro-2H-pyran-4-yloxy)imidazo[1,2-b]pyridazin-3-yl)phenol;
4-(2-amino-6-(tetrahydro-2H-pyran-4-yloxy)imidazo[1,2-b]pyridazin-3-yl)benzonitrile;
(E)-methyl 3-(3-(6-(tetrahydro-2H-pyran-4-ylamino)imidazo[1,2-b]pyridazin-3-yl)phenyl)acrylate;
(E)-3-(3-(6-(tetrahydro-2H-pyran-4-ylamino)imidazo[1,2-b]pyridazin-3-yl)phenyl)acrylic acid;
6-(cyclohexylthio)-3-(4-fluorophenyl)imidazo[1,2-b]pyridazine;
3-(1,4-dioxaspiro[4.5]dec-7-en-8-yl)-N-(tetrahydro-2H-pyran-4-yl)imidazo[1,2-b]-pyridazin-6-amine;
tert-butyl 2-(6-(tetrahydro-2H-pyran-4-ylamino)imidazo[1,2-b]pyridazin-3-yl)-1H-pyrrole-1-carboxylate;
3-(1H-pyrrol-2-yl)-N-(tetrahydro-2H-pyran-4-yl)imidazo[1,2-b]pyridazin-6-amine;
3-(4-(2H-1,2,3-triazol-4-yl)phenyl)-N-(tetrahydro-2H-pyran-4-yl)imidazo[1,2-b]-pyridazin-6-amine;
3-(4-(2H-tetrazol-5-yl)phenyl)-N-(tetrahydro-2H-pyran-4-yl)imidazo[1,2-b]pyridazin-6-amine;
(E)-3-(3-(6-(tetrahydro-2H-pyran-4-ylamino)imidazo[1,2-b]pyridazin-3-yl)phenyl)acrylamide;
3-(4-fluorophenyl)-6-(methylthio)imidazo[1,2-b]pyridazine;
4-(6-(tetrahydro-2H-pyran-4-ylamino)imidazo[1,2-b]pyridazin-3-yl)benzimidamide;
5-(6-(tetrahydro-2H-pyran-4-ylamino)imidazo[1,2-b]pyridazin-3-yl)thiophene-2-carbaldehyde;
3-(4-fluorophenyl)imidazo[1,2-b]pyridazin-6-amine;
3-(4-(pent-1-ynyl)phenyl)-N-(tetrahydro-2H-pyran-4-yl)imidazo[1,2-b]pyridazin-6-amine;
3-(1H-indazol-6-yl)-N-(tetrahydro-2H-pyran-4-yl)imidazo[1,2-b]pyridazin-6-amine;
3-(benzofuran-5-yl)-N-(tetrahydro-2H-pyran-4-yl)imidazo[1,2-b]pyridazin-6-amine;
N-(2-(dimethylamino)ethyl)-3-(6-(tetrahydro-2H-pyran-4-ylamino)imidazo[1,2-b]-pyridazin-3-yl)benzamide;
(4-methylpiperazin-1-yl)(3-(6-(tetrahydro-2H-pyran-4-ylamino)imidazo[1,2-b]pyridazin-3-yl)phenyl)methanone;
(4-methylpiperazin-1-yl)(4-(6-(tetrahydro-2H-pyran-4-ylamino)imidazo[1,2-b]pyridazin-3-yl)phenyl)methanone;
(E)-N-(tetrahydro-2H-pyran-4-yl)-3-(2-(trimethylsilyl)vinyl)imidazo[1,2-b]pyridazin-6-amine;
(4-(6-(tetrahydro-2H-pyran-4-ylamino)imidazo[1,2-b]pyridazin-3-yl)phenyl)methanol;
3-(3-(2-chlorobenzyloxy)phenyl)-N-(tetrahydro-2H-pyran-4-yl)imidazo[1,2-b]pyridazin-6-amine;
(E)-3-(oct-1-enyl)-N-(tetrahydro-2H-pyran-4-yl)imidazo[1,2-b]pyridazin-6-amine;
4-(6-(tetrahydro-2H-pyran-4-ylamino)imidazo[1,2-b]pyridazin-3-yl)benzamide;
2-fluoro-4-(6-(tetrahydro-2H-pyran-4-ylamino)imidazo[1,2-b]pyridazin-3-yl)benzohydrazide;
2-(4-(6-(tetrahydro-2H-pyran-4-ylamino)imidazo[1,2-b]pyridazin-3-yl)phenyl)acetonitrile;
N-(4-(6-(tetrahydro-2H-pyran-4-ylamino)imidazo[1,2-b]pyridazin-3-yl)phenyl)methanesulfonamide;
(S)-2-amino-3-(4-(6-(tetrahydro-2H-pyran-4-ylamino)imidazo[1,2-b]pyridazin-3-yl)phenyl)propanoic acid;
(E)-3-(pent-1-enyl)-N-(tetrahydro-2H-pyran-4-yl)imidazo[1,2-b]pyridazin-6-amine;
3-(4-methyl-3,4-dihydro-2H-benzo[b][1;4]oxazin-6-yl)-N-(tetrahydro-2H-pyran-4-yl)imidazo[1,2-b]pyridazin-6-amine;
3-(4-ethylphenyl)-N-(tetrahydro-2H-pyran-4-yl)imidazo[1,2-b]pyridazin-6-amine;
5-(6-(tetrahydro-2H-pyran-4-ylamino)imidazo[1,2-b]pyridazin-3-yl)thiophene-2-carboxylic acid;
(E)-3-styryl-N-(tetrahydro-2H-pyran-4-yl)imidazo[1,2-b]pyridazin-6-amine;
3-(5-chlorothiophen-2-yl)-N-(tetrahydro-2H-pyran-4-yl)imidazo[1,2-b]pyridazin-6-amine;
3-(5-methylthiophen-2-yl)-N-(tetrahydro-2H-pyran-4-yl)imidazo[1,2-b]pyridazin-6-amine;
3-(2,4-difluorophenyl)-N-(tetrahydro-2H-pyran-4-yl)imidazo[1,2-b]pyridazin-6-amine;
3-(3,4-difluorophenyl)-N-(tetrahydro-2H-pyran-4-yl)imidazo[1,2-b]pyridazin-6-amine;
3-(4-tert-butylphenyl)-N-(tetrahydro-2H-pyran-4-yl)imidazo[1,2-b]pyridazin-6-amine;
N-(2-hydroxyethyl)-4-(6-(tetrahydro-2H-pyran-4-ylamino)imidazo[1,2-b]pyridazin-3-yl)benzenesulfonamide;
4-(6-(tetrahydro-2H-pyran-4-ylamino)imidazo[1,2-b]pyridazin-3-yl)-N-(1H-tetrazol-5-yl)benzamide; or
4-(6-(tetrahydro-2H-pyran-4-ylamino)imidazo[1,2-b]pyridazin-3-yl)benzenesulfonamide.

36. A method of treating an IRAK-responsive condition or disorder in a subject, comprising administering to the subject in need of such treatment a therapeutically effective amount of a compound of Formula (I) as described in claim 1, wherein the condition or disorder is rheumatoid arthritis, multiple sclerosis, sepsis, osteoarthritis, inflammatory bowel disease, osteoporosis, myasthenia gravis, stroke, Alzheimer's disease, Parkinson's disease, amyotrophic lateral sclerosis, psoriasis, cardiac contractile dysfunction, type I diabetes, type II diabetes, familial cold autoinflammatory syndrome, or severe bacterial infections.

37.-45. (canceled)
